SEQUENCE LISTING (12 sequences)

SEQ ID NO: 1 - DNA, 498 bp, Homo sapiens
  misc_feature (1)..(498): Human osteocalcin cDNA
cgcagccaccgagacaccatgagagccctcacactcctcgccctattggccctggccgcactttgcatcgctggccaggcaggtgcgaagcccagcggtgcagagtccagcaaaggtgcagcctttgtgtccaagcaggagggcagcgaggtagtgaagagacccaggcgctacctgtatcaatggctgggagccccagtcccctacccggatcccctggagcccaggagggaggtgtgtgagctcaatccggactgtgacgagttggctgaccacatcggctttcaggaggcctatcggcgcttctacggcccggtctagggtgtcgctctgctggcctggccggcaaccccagttctgctcctctccaggcacccttctttcctcttccccttgcccttgccctgacctcccagccctatggatgtggggtccccatcatcccagctgctcccaaataaactccagaagaggaatctgaaaaaaaaaaaaaaaaaa

SEQ ID NO: 2 - protein, 100 aa, Homo sapiens
  misc_feature (1)..(100): Pre-pro-sequence of human osteocalcin
MRALTLLALLALAALCIAGQAGAKPSGAESSKGAAFVSKQEGSEVVKRPRRYLYQWLGAPVPYPDPLEPRREVCELNPDCDELADHIGFQEAYRRFYGPV

SEQ ID NO: 3 - DNA, 494 bp, Mus musculus
  misc_feature (1)..(494): Mouse osteocalcin gene 1 cDNA
agaacagacaagtcccacacagcagcttggcccagacctagcagacaccatgaggaccatctttctgctcactctgctgaccctggctgcgctctgtctctctgacctcacagatgccaagcccagcggccctgagtctgacaaagccttcatgtccaagcaggagggcaataaggtagtgaacagactccggcgctaccttggagcctcagtccccagcccagatcccctggagcccacccgggagcagtgtgagcttaaccctgcttgtgacgagctatcagaccagtatggcttgaagaccgcctacaaacgcatctatggtatcactatttaggacctgtgctgccctaaagccaaactctggcagctcggctttggctgctctccgggacttgatcctccctgtcctctctctctgccctgcaagtatggatgtcacagcagctccaaaataaagttcagatgaggaagtgcaaaaaaaaaaaaaaaaa

SEQ ID NO: 4 - DNA, 470 bp, Mus musculus
  misc_feature (1)..(470): Mouse osteocalcin gene 2 cDNA
gaacagacaagtcccacacagcagcttggtgcacacctagcagacaccatgaggaccctctctctgctcactctgctggccctggctgcgctctgtctctctgacctcacagatcccaagcccagcggccctgagtctgacaaagccttcatgtccaagcaggagggcaataaggtagtgaacagactccggcgctaccttggagcctcagtccccagcccagatcccctggagcccacccgggagcagtgtgagcttaaccctgcttgtgacgagctatcagaccagtatggcttgaagaccgcctacaaacgcatctacggtatcactatttaggacctgtgctgccctaaagccaaactctggcagctcggctttggctgctctccgggacttgatcctccctgtcctctctctctgccctgcaagtatggatgtcacagcagctccaaaataaagttcagatgagg

SEQ ID NO: 5 - protein, 95 aa, Mus musculus
  misc_feature (1)..(95): amino acid sequence encoded by mouse osteocalcin gene 1 and gene 2
MRTLSLLTLLALAALCLSDLTDPKPSGPESDKAFMSKQEGNKVVNRLRRYLGASVPSPDPLEPTREQCELNPACDELSDQYGLKTAYKRIYGITI

SEQ ID NO: 6 - protein, 1215 aa, Homo sapiens
  misc_feature (1)..(1215): epitope in human GPR158
MGAMAYPLLLCLLLAQLGLGAVGASRDPQGRPDSPRERTPKGKPHAQQPGRASASDSSAPWSRSTDGTILAQKLAEEVPMDVASYLYTGDSHQLKRANCSGRYELAGLPGKWPALASAHPSLHRALDTLTHATNFLNVMLQSNKSREQNLQDDLDWYQALVWSLLEGEPSISRAAITFSTDSLSAPAPQVFLQATREESRILLQDLSSSAPHLANATLETEWFHGLRRKWRPHLHRRGPNQGPRGLGHSWRRKDGLGGDKSHFKWSPPYLECENGSYKPGWLVTLSSAIYGLQPNLVPEFRGVMKVDINLQKVDIDQCSSDGWFSGTHKCHLNNSECMPIKGLGFVLGAYECICKAGFYHPGVLPVNNFRRRGPDQHISGSTKDVSEEAYVCLPCREGCPFCADDSPCFVQEDKYLRLAIISFQALCMLLDFVSMLVVYHFRKAKSIRASGLILLETILFGSLLLYFPVVILYFEPSTFRCILLRWARLLGFATVYGTVTLKLHRVLKVFLSRTAQRIPYMTGGRVMRMLAVILLVVFWFLIGWTSSVCQNLEKQISLIGQGKTSDHLIFNMCLIDRWDYMTAVAEFLFLLWGVYLCYAVRTVPSAFHEPRYMAVAVHNELIISAIFHTIRFVLASRLQSDWMLMLYFAHTHLTVTVTIGLLLIPKFSHSSNNPRDDIATEAYEDELDMGRSGSYLNSSINSAWSEHSLDPEDIRDELKKLYAQLEIYKRKKMITNNPHLQKKRCSKKGLGRSIMRRITEIPETVSRQCSKEDKEGADHGTAKGTALIRKNPPESSGNTGKSKEETLKNRVFSLKKSHSTYDHVRDQTEESSSLPTESQEEETTENSTLESLSGKKLTQKLKEDSEAESTESVPLVCKSASAHNLSSEKKTGHPRTSMLQKSLSVIASAKEKTLGLAGKTQTAGVEERTKSQKPLPKDKETNRNHSNSDNTETKDPAPQNSNPAEEPRKPQKSGIMKQQRVNPTTANSDLNPGTTQMKDNFDIGEVCPWEVYDLTPGPVPSESKVQKHVSIVASEMEKNPTFSLKEKSHHKPKAAEVCQQSNQKRIDKAEVCLWESQGQSILEDEKLLISKTPVLPERAKEENGGQPRAANVCAGQSEELPPKAVASKTENENLNQIGHQEKKTSSSEENVRGSYNSSNNFQQPLTSRAEVCPWEFETPAQPNAGRSVALPASSALSANKIAGPRKEEIWDSFKV

SEQ ID NO: 7 - DNA, 6663 bp, Artificial Sequence
  Synthetic
tccaaatttaaaaagtgattcccccccctcccgttccctcctcttctctctgggaggcagatgggagccatggcttaccccttactcctctgcctcctgcttgctcagctgggattgggagctgttggcgccagccgcgacccccaaggacggccggattcccctcgagagaggaccccgaaggggaagccgcacgcccagcagccgggtcgagcctctgcctcggactcctcggctccctggagccgctccaccgatggcaccatcttggcgcagaaactcgccgaggaggtgcccatggacgtggcctcttacctctacaccggggactcccaccagctgaagcgagccaactgctccggccgctacgagttggcgggcctgccggggaagtggccagccctggccagcgcgcacccctccttgcaccgggcgctggacacactgacacacgccaccaacttcctcaacgtgatgctgcagagcaataagtcgcgggagcagaacttgcaggacgacctggattggtaccaggcgctggtgtggagccttctggagggcgagcccagcatctcccgggcggccatcaccttcagcaccgattcgctgtccgcaccggccccacaggtcttcctccaggccacgcgcgaggagagccgcatcctgctccaagacctgtcctcctccgcaccccacctggccaacgccactctggagaccgagtggttccacggcctccggcgcaagtggaggccccacttacaccgccgcggccccaatcaggggccccggggcctgggccacagctggcggcgcaaggacgggctcggcggggacaagagccacttcaagtggtctccgccttatctggagtgcgagaacgggagttacaagcccgggtggctggttactctttcctctgccatctacgggttgcagcctaacctggtcccggaattcaggggtgtcatgaaagttgacataaatcttcagaaagtggacattgaccaatgctcaagtgatggctggttttcaggaactcataaatgccacctcaacaattcagagtgtatgccaattaaaggcctaggattcgttcttggagcctatgagtgcatttgcaaagcaggattctatcatcctggagtcttaccagtgaacaactttcggagaaggggtccggatcagcatatttcaggaagtacaaaagatgtgtcagaagaagcctatgtctgcctaccttgcagggagggctgccccttctgtgctgatgacagcccatgcttcgtccaggaagataagtatttacgacttgccatcatctccttccaagccctgtgtatgctgctcgacttcgttagcatgctggtggtctaccactttcgcaaagcaaagagcatccgggcatcgggccttatcctgttggaaacgatcctttttggatctctgctcctatactttccagttgttattttgtactttgagccaagcacatttcgctgtattctcctaagatgggctcgtcttctcggttttgctactgtttacggaactgtcactctcaaacttcacagggttttgaaggtgtttctttcacgaacggctcaacgaattccatatatgactggcggacgggtcatgaggatgctggcagtaatactcttggtagtgttttggtttctcattggctggacttcatctgtgtgccagaatttggagaaacagatttcacttattggccaggggaaaacatccgatcacctcatcttcaatatgtgcctcattgaccgctgggactacatgacagcagttgctgaatttttattcctcttgtggggtgtttatctctgctatgcagtgcggacagtcccatcggcattccatgagccccgctatatggctgttgcagttcacaatgagctcatcatctctgctatattccatacaattagatttgttcttgcctcaagacttcagtctgattggatgttgatgctgtattttgcacatactcatttgactgtgacagtcaccattgggttgcttttgattccaaagttttcacattcaagcaataacccacgagatgatattgctacagaagcatatgaggatgagctagacatgggccgatctggatcctacctgaacagcagtatcaattcagcctggagtgagcacagcttggatccagaggacattcgggacgagctgaaaaaactctatgcccaactggaaatatataaaagaaagaagatgatcacaaacaacccccacctccagaaaaagcggtgctcgaagaagggcctaggtcgttccatcatgagacgcattacggagatcccagagacagtcagccggcagtgctctaaagaggacaaggagggcgccgaccatggcacagccaaaggcactgccctcatcaggaagaaccccccagagtcttcagggaacacagggaaatccaaggaggagaccctgaaaaaccgagtcttctcactcaagaaatcccacagcacttatgaccacgtgagagaccaaacggaagagtccagtagcctacccacagaaagccaagaggaggagacaacagaaaattccacactggaatccctgtcgggtaaaaaactaacacaaaaactaaaagaagacagcgaggctgagtccacggagtcggtgccgttggtgtgcaagtcagcaagcgctcacaacctcagctcagagaagaaaactgggcacccacgaacatcgatgttacagaagtctctcagtgtcatagcaagcgccaaggagaagactcttggattagctgggaaaacccaaacagcaggtgtggaagaacgcactaaatcccagaaacctttgccaaaagataaagagacaaacagaaatcactcaaattctgataacacagagactaaagatcctgccccccaaaactcaaatcctgcggaggagccaagaaagcctcagaaatctgggattatgaaacaacaaagggtcaaccccaccactgccaattctgacctgaacccaggcaccacccagatgaaggacaactttgacattggggaggtgtgtccttgggaggtttatgacctgacccctggtcctgtgccttcagaatcaaaagttcaaaagcacgtatctattgtggcttctgaaatggagaaaaaccccactttttccttaaaggagaaatctcaccacaagcctaaggcagctgaggtttgtcagcaatccaatcagaagcgcatagataaggctgaagtatgcctttgggagagccaaggccagtccattttggaagatgagaagcttttgatttccaagactccagttctcccagagagggcaaaagaggagaacggaggtcagcctcgtgcagccaatgtgtgtgctgggcagagcgaagaactgccccccaaagctgtagcatcaaaaacagagaatgaaaatctcaaccaaataggacaccaggaaaaaaagacatcttcttctgaggagaatgtgcgtggctcctataactcaagtaataacttccagcaacctttaacatcacgagcagaggtttgtccttgggagtttgagaccccagctcaaccaaatgctggaagaagtgtagctttacctgcctcttctgctctaagtgcaaataagatagcagggcctaggaaagaagagatctgggatagttttaaagtgtagcatctccaggaagaagaggaaaaggagggaaccccggattggatatgagacagaagatataagaatcaaatattcccaaggaggatttgtcaatcaaggaaaacatgacagatggtgaggtaaagtcaaaggcatgggtagaagaggaccaggggggcaagagcaacaacgtcataatggagaagtcagactttggtcaagaaagtccttcccttggtaacactaggaaaatctttccatttcagcatgtttaaggaaaatagcccacaatgtctgccctgatcaatatgtatccatgggactttgaagatcctaagccaggtaaaccaggagacacagaagacgtaccagatttgcaaagaaagaaaaggtataagacatatataactgaaattctaagtagctgaccgagaagaacttactttacctatttaaccttgatagcactgctaacttaatgcatcccaaaaatatcttttatattaatgattgctctcattttcttataaatgtatgtttcagtatatcgttgtgtctcatattcaagcattccagattgtataatttttgcaaataactttggtattatgtgacacaacacatttatgcaatctgcagctattcaattgttattgcaccttacagaatacctgctatctatcaactttagttgattcttgaagtacagtaagctttctctggcttgggaagccataactgttactataaaaacttttagttttggctgtggtttatatattgtgactttgaatttgactctattatttcacatcatggtttgttatactgtcttaatcagggttttttatacaagttgagttacttgttttgcacttcttgttaggactcagaagctttattaatattggagatcaagtggtcctacttagtcatatgtctcaataagttaaggacaacttatccgttgtttattcaaagtcagagatagataacgccttcattccaattaattgtcccttttaactctttcagtatttcctacttagcagcatttccaaaggaagaagctaagagtgagaaaaatataccgtgcattattattactattggaaagggaagactctagggatgacataagaattatagcagtactataaacccaggaagtttgcctttcaaaaaaaaacacaggtagctcctgatagcactttcaagggattatttttttaaagagaaaaattatggtagcatcaagatcattgtatggatatatttttattatgtgtactgaaaatacagtattttaaaataccttaaagtatttattctcataaactcttattcattgcttcagctacaggtagaacttgctgggctcaaatcccaaagaggttttataaccttatttattcaaaacctataaggtggtatggaatcttcattctcccaagcactggaaaatgtctaagtcctgcaaattgccattgtgagccacttgctcgacatgtaacatgtaaggtccatttgcaaagcaaagcagcccccaaagcatattttataaagcttattgcattccacactgatctcttggcatgggaatcctaagctgccgactaagccctaccgacatgatcatgcagtgagattccaaagctcagtctcatttcatttaaagaagaatcactcagaaatagaaccgagacttccctttttctccctgtaaacacccaagtatcaactgcttatttggccaggacactcccagcacaaataactattttttatgtcacaagcagcaaggaggacatgctagggtgataaaagatggagaaacaggatcagagggtggatatagggctgttcttagagagtattttcagtggaaggtaaaaacagaattctccatattcatcatcaaatttttctcagtgatttttttattcaggagtaagcaagcacttcactgtttcacaaagctgtgcgcaaatcttcctcacccatttgctgactttatgcattactcaggttggtggggtcggtttgagaagatatagaaattctatttttgtgtctttacaccatttatttcttttatctcttccttttcaatgaaggcctatatgcttggtgacctcctttaaggaatctttgtgaactgggttggaagttcctagacccacatatttgtttcatttatgtctgaaatctgttagcacttgattcctttcttgagaattatgcagtcaagcatcagtgactttctattgcacttcaggattgatcctgctagagatgtgagttaaaaagacttgccaaattatatcttagcgacattctatagttcatagattattctccaccagcataaatcagtgagagtgcctagagtctttctgagagtttcattgccattatcaacaagagaagttgaaatttacaagtcaggaggttatttttccagattgataaccatagaaagtgaataaacacttttaaggtcgcaaacatttgctaggttgtccttctcaatgcatgtgcaggctgcatcctgtccttgtttttaagccagggtttataaataagtagatttataccaatcttaatagaattgtatattttatgcaagaattaaatgctttacaacatgaagtataactcaacccattgtaaactttggtggcaatatggatttgaaactcgacagttctcttgtatttgcttcctaggtttctgcatgcaagttatgacaggtaggactgaaaaaacactgccttttgacttctagcatttagcaaccgagagtcgtagagtcaataaagctgtaagtgtcttcacttaatctgtggttctcctaaaactattatctgaaacctacagcatcccaccatgaaatatttggtaaatttatgttgtgacgtgttgcagcatgtaaataattataacttctctgcaataaaacatatttatatgaaa

SEQ ID NO: 8 - protein, 1215 aa, Artificial Sequence; identical to SEQ ID NO: 6
  Synthetic

SEQ ID NO: 9 - protein, 49 aa, Homo sapiens
  misc_feature (1)..(49): Mature human osteocalcin protein
YLYQWLGAPVPYPDPLEPRREVCELNPDCDELADHIGFQEAYRRFYGPV

SEQ ID NO: 10 - protein, 49 aa, Unknown
  Osteocalcin variant
  misc_feature: If each Xaa is glutamic acid, then Xaa at position 17 is not carboxylated, or less than 50 percent of Xaa at position 21 is carboxylated, and/or less than 50 percent of Xaa at position 24 is carboxylated
  misc_feature (17)..(17): Xaa can be any naturally occurring amino acid
  misc_feature (21)..(21): Xaa can be any naturally occurring amino acid
  misc_feature (24)..(24): Xaa can be any naturally occurring amino acid
YLYQWLGAPVPYPDPLXPRRXVCXLNPDCDELADHIGFQEAYRRFYGPV

SEQ ID NO: 11 - DNA, 2860 bp, Homo sapiens
  misc_feature (1)..(2860): Nucleotide sequence encoding human GPRC6A
actgagcaaatgagatagaaacatggcattcttaattatactaattacctgctttgtgattattcttgctacttcacagccttgccagacccctgatgactttgtggctgccacttctccgggacatatcataattggaggtttgtttgctattcatgaaaaaatgttgtcctcagaagactctcccagacgaccacaaatccaggagtgtgttggctttgaaatatcagtttttcttcaaactcttgccatgatacacagcattgagatgatcaacaattcaacactcttatctggagtcaaactggggtatgaaatctatgacacttgtacagaagtcacagtggcaatggcagccactctgaggtttctttctaaattcaactgctccagagaaactgtggagtttaagtgtgactattccagctacatgccaagagttaaggctgtcataggttctgggtactcagaaataactatggctgtctccaggatgttgaatttacagctcatgccacaggtgggttatgaatcaactgcagaaatcctgagtgacaaaattcgctttccttcatttttacggactgtgcccagtgacttccatcaaattaaagcaatggctcacctgattcagaaatctggttggaactggattggcatcataaccacagatgatgactatggacgattggctcttaacacttttataattcaggctgaagcaaataacgtgtgcatagccttcaaagaggttcttccagcctttctttcagataataccattgaagtcagaatcaatcggacactgaagaaaatcattttagaagcccaggttaatgtcattgtggtatttctgaggcaattccatgtttttgatctcttcaataaagccattgaaatgaatataaataagatgtggattgctagtgataattggtcaactgccaccaagattaccaccattcctaatgttaaaaagattggcaaagttgtagggtttgcctttagaagagggaatatatcctctttccattcctttcttcaaaatctgcacttgcttcccagtgacagtcacaaactcttacatgaatatgccatgcatttatctgcctgcgcatatgtcaaggacactgatttgagtcaatgcatattcaatcattctcaaaggactttggcctacaaggctaacaaggctatagaaaggaacttcgtcatgagaaatgacttcctctgggactatgctgagccaggactcattcatagtattcagcttgcagtgtttgcccttggttatgccattcgggatctgtgtcaagctcgtgactgtcagaaccccaacgcctttcaaccatgggagttacttggtgtgctaaaaaatgtgacattcactgatggatggaattcatttcattttgatgctcacggggatttaaatactggatatgatgttgtgctctggaaggagatcaatggacacatgactgtcactaagatggcagaatatgacctacagaatgatgtcttcatcatcccagatcaggaaacaaaaaatgagttcaggaatcttaagcaaattcaatctaaatgctccaaggaatgcagtcctgggcaaatgaagaaaactacaagaagtcaacacatctgttgctatgaatgtcagaactgtcctgaaaatcattacactaatcagacagatatgcctcactgccttttatgcaacaacaaaactcactgggcccctgttaggagcactatgtgctttgaaaaggaagtggaatatctcaactggaatgactccttggccatcctactcctgattctctccctactgggaatcatatttgttctggttgttggcataatatttacaagaaacctgaacacacctgttgtgaaatcatccgggggattaagagtctgctatgtgatccttctctgtcatttcctcaattttgccagcacgagctttttcattggagaaccacaagacttcacatgtaaaaccaggcagacaatgtttggagtgagctttactctttgcatctcctgcattttgacgaagtctctgaaaattttgctagccttcagctttgatcccaaattacagaaatttctgaagtgcctctatagaccgatccttattatcttcacttgcacgggcatccaggttgtcatttgcacactctggctaatctttgcagcacctactgtagaggtgaatgtctccttgcccagagtcatcatcctggagtgtgaggagggatccatacttgcatttggcaccatgctgggctacattgccatcctggccttcatttgcttcatatttgctttcaaaggcaaatatgagaattacaatgaagccaaattcattacatttggcatgctcatttacttcatagcttggatcacattcatccctatctatgctaccacatttggcaaatatgtaccagctgtggagattattgtcatattaatatctaactatggaatcctgtattgcacattcatccccaaatgctatgttattatttgtaagcaagagattaacacaaagtctgcctttctcaagatgatctacagttattcttcccatagtgtgagcagcattgccctgagtcctgcttcactggactccatgagcggcaatgtcacaatgaccaatcccagctctagtggcaagtctgcaacctggcagaaaagcaaagatcttcaggcacaagcatttgcacacatatgcagggaaaatgccacaagtgtatctaaaactttgcctcgaaaaagaatgtcaagtatatgaataagccttaggagatgccacattccagaataaaatgtttccagggtctttgcatct

SEQ ID NO: 12 - protein, 926 aa, Homo sapiens
  misc_feature (1)..(926): Amino acid sequence of human GPRC6A
MAFLIILITCFVIILATSQPCQTPDDFVAATSPGHIIIGGLFAIHEKMLSSEDSPRRPQIQECVGFEISVFLQTLAMIHSIEMINNSTLLSGVKLGYEIYDTCTEVTVAMAATLRFLSKFNCSRETVEFKCDYSSYMPRVKAVIGSGYSEITMAVSRMLNLQLMPQVGYESTAEILSDKIRFPSFLRTVPSDFHQIKAMAHLIQKSGWNWIGIITTDDDYGRLALNTFIIQAEANNVCIAFKEVLPAFLSDNTIEVRINRTLKKIILEAQVNVIVVFLRQFHVFDLFNKAIEMNINKMWIASDNWSTATKITTIPNVKKIGKVVGFAFRRGNISSFHSFLQNLHLLPSDSHKLLHEYAMHLSACAYVKDTDLSQCIFNHSQRTLAYKANKAIERNFVMRNDFLWDYAEPGLIHSIQLAVFALGYAIRDLCQARDCQNPNAFQPWELLGVLKNVTFTDGWNSFHFDAHGDLNTGYDVVLWKEINGHMTVTKMAEYDLQNDVFIIPDQETKNEFRNLKQIQSKCSKECSPGQMKKTTRSQHICCYECQNCPENHYTNQTDMPHCLLCNNKTHWAPVRSTMCFEKEVEYLNWNDSLAILLLILSLLGIIFVLVVGIIFTRNLNTPVVKSSGGLRVCYVILLCHFLNFASTSFFIGEPQDFTCKTRQTMFGVSFTLCISCILTKSLKILLAFSFDPKLQKFLKCLYRPILIIFTCTGIQVVICTLWLIFAAPTVEVNVSLPRVIILECEEGSILAFGTMLGYIAILAFICFIFAFKGKYENYNEAKFITFGMLIYFIAWITFIPIYATTFGKYVPAVEIIVILISNYGILYCTFIPKCYVIICKQEINTKSAFLKMIYSYSSHSVSSIALSPASLDSMSGNVTMTNPSSSGKSATWQKSKDLQAQAFAHICRENATSVSKTLPRKRMSSI

(19) United States
(12) Patent Application Publication
(10) Pub. No.: US 2020/0069775 A1
(43) Pub. Date: Mar. 5, 2020

(54) BRAIN OSTEOCALCIN RECEPTOR AND COGNITIVE DISORDERS

(71) Applicant: The Trustees of Columbia University in the City of New York, New York, NY (US)

(72) Inventors: Gerard KARSENTY, New York, NY (US); Arnaud OBRI, New York, NY (US); Lori KHRIMIAN, Sunnyside, NY (US)

(21) Appl. No.: 16/486,428

(22) PCT Filed: Feb. 15, 2018
(86) PCT No.: PCT/US2018/018311

§ 371 Date: Aug. 15, 2019

Related U.S. Application Data
(60) Provisional application No. 62/459,329, filed on Feb. 15, 2017.

(51) Int. Cl. A61K 38/23; A61P 25/28; G01N 33/68

(52) U.S. Cl. CPC A61K 38/23; A61P 25/28; G01N 2800/2814; G01N 2333/575; G01N 33/6896

(57) ABSTRACT

Methods and compositions for treating or preventing cognitive disorders in mammals, preferably humans, are provided. The methods generally involve activation of the GPR158 signaling pathway involving osteocalcin, e.g., by administratin of undercarboxylated/uncarboxylated osteocalcin. Disorders amenable to treatment by the methods include, but are not limited to, cognitive loss due to neurodegeneration associated with aging, anxiety, depression, memory loss, learning difficulties, and cognitive disorders associated with food deprivation during pregnancy.

### CROSS-REFERENCE TO RELATED APPLICATION(S)

This application claims priority from U.S. Provisional Application No. 62/459,329, filed Feb. 15, 2017, the entire disclosure of which is incorporated herein by reference.

### STATEMENT OF GOVERNMENT INTEREST

This disclosure was made with Government support under grant 2P01AG032959-06A1 awarded by the National Institutes of Health/National Institute on Aging. The government has certain rights in the invention.

### FIELD OF THE DISCLOSURE

The present disclosure is directed to methods and compositions for treating or preventing cognitive disorders in mammals. Such cognitive disorders include, but are not limited to, cognitive loss due to neurodegeneration associated with aging, anxiety, depression, memory loss, learning difficulties, and cognitive disorders associated with food deprivation during pregnancy.

### BACKGROUND INFORMATION

Osteocalcin, one of the very few osteoblast-specific proteins, has several features of a hormone. For instance, it is synthesized as a pre-pro-molecule and is secreted in the general circulation (Hauschka et al., 1989, Physiol. Review 69:990-1047; Price, 1989, Connect. Tissue Res. 21:51-57 (discussion 57-60)). Because of their exquisite cell-specific expression, the osteocalcin genes have been intensively studied to identify osteoblast-specific transcription factors and to define the molecular bases of bone physiology (Ducy et al., 2000, Science 289:1501-1504; Harada & Rodan, 2003, Nature 423:349-355).

Osteocalcin is the most abundant non-collagenous protein found associated with the mineralized bone matrix and it is currently being used as a biological marker for clinical assessment of bone turnover. Osteocalcin is a small (46-50 amino acid residues) bone specific protein that contains 3 gamma-carboxylated glutamic acid residues in its primary structure. The name osteocalcin (osteo, Greek for bone; calc, Latin for lime salts; in, protein) derives from the protein's ability to bind Ca2+ and its abundance in bone. Osteocalcin undergoes a peculiar post-translational modification whereby glutamic acid residues are carboxylated to form gamma-carboxyglutamic acid (Gla) residues; hence osteocalcin's other name, bone Gla protein (Hauschka et al., 1989, Physiol. Review 69:990-1047).

Osteocalcin binds to neurons in the midbrain and hippocampus, regulates neurotransmitter synthesis, reduces anxiety, and promotes memory (Oury, F. et al., 2013, Cell 155:228-241). The severity of the behavioral defects observed in Osteocalcin−/− mice, together with the steep decrease in circulating osteocalcin levels before mid-life both in mice and humans (Mera, P. et al., 2016, Cell Metab 23:1078-1092) raises the question of whether and how changes in bone health over time may contribute to the age-related decline in cognitive functions.

Mature human osteocalcin contains 49 amino acids with a predicted molecular mass of 5,800 kDa (Poser et al., 1980, J. Biol. Chem. 255:8685-8691). Osteocalcin is synthesized primarily by osteoblasts and ondontoblasts and comprises 15 to 20% of the non-collagenous protein of bone. Poser et al., 1980, J. Biol. Chem. 255:8685-8691 showed that mature osteocalcin contains three carboxyglutamic acid residues which are formed by post-translational vitamin K-dependent modification of glutamic acid residues. The carboxylated Gla residues are at positions 17, 21 and 24 of mature human osteocalcin. Some human osteocalcin has been shown to contain only 2 Gla residues (Poser & Price, 1979, J. Biol. Chem. 254:431-436).

Osteocalcin has several features of a hormone. Ducy et al., 1996, Nature 382:448-452 demonstrated that mineralized bone from aging osteocalcin-deficient mice was two times thicker than that of wild-type. It was shown that the absence of osteocalcin led to an increase in bone formation without impairing bone resorption and did not affect mineralization. Multiple immunoreactive forms of human osteocalcin have been discovered in circulation (Garnero et al., 1994, J. Bone Miner. Res. 9:255-264) and also in urine (Taylor et al., 1990, J. Clin. Endocrin. Metab. 70:467-472). Fragments of human osteocalcin can be produced either during osteoclastic degradation of bone matrix or as the result of the catabolic breakdown of the circulating protein after synthesis by osteoblasts.

The identification in recent years of novel organs influencing bone physiology expanded the spectrum of questions studied in skeletal biology. An example of this is the regulation of bone mass accrual by the brain that was first revealed by studying the mechanisms whereby the adipocyte-derived hormone leptin decreases bone mass accrual in all species tested (Ducy et al., 2000, Cell 100:197-207; Pogoda et al., 2006, J. Bone and Mineral Res. 21:1591-1599; Elefteriou et al., 2004, Proceedings of the National Academy of Sciences of the United States of America 101:3258-3263; Vaira et al., 2012, Neuroscience Biobehavioral Rev. 29:237-258). The use of cell-specific gene deletion models revealed widespread evidence that leptin signals in brainstem neurons to prevent synthesis of serotonin, a neurotransmitter that decreases the activity of the sympathetic nervous system, an inhibitor of bone mass accrual (Takeda et al., 2002, Cell 111:305-317; Yadav et al., 2009, Cell 138:976-989; Oury et al., 2010, Genes & Development 24:2330-2342, Genes Dev. 24:2330-2342). What underlines best the importance of this function of brain-derived serotonin is the fact that selective serotonin reuptake inhibitors (SSRIs) that increase the local concentrations of serotonin in the brain (Gardier et al., 1996, Fundamental Clin. Pharmacol. 10:16-27) have deleterious effects on bone mass in humans.

A second development of significance in skeletal biology has been the demonstration that bone is an endocrine organ secreting at least two hormones. One of them, osteocalcin, is made by the osteoblast, the bone forming cell, and promotes several functions apparently unrelated to bone health such as energy expenditure, insulin secretion, insulin sensitivity, and, in males, testosterone synthesis (Lee et al., 2007, Cell 130:456-469; Oury et al., 2011, Cell 144:796-809). The latter function occurs following the binding of osteocalcin to a specific receptor, gprc6a, on Leydig cells (Oury et al., 2011, Cell 144:796-809).

OST-PTP is the protein encoded by the Esp gene. The Esp gene was originally named for embryonic stem (ES) cell phosphatase and it has also been called the Ptpry gene in mice. (Lee et al, 1996, Mech. Dev. 59:153-164). Because of its bone and testicular localization, the gene product of Esp is often referred to as osteoblast testicular protein tyrosine phosphatase (OST-PTP). OST-PTP is a large, 1,711 amino acid-long protein that includes three distinct domains. OST-PTP has a 1,068 amino-acid long extracellular domain containing multiple fibronectin type III repeats. Gprc6a is a receptor that belongs to the C family of GPCRs (Wellendorph and Brauner-Osborne, 2004, Gene 335:37-46) and has been proposed to be a receptor for amino acids or for calcium in the presence of osteocalcin as a cofactor, and for androgens (Pi et al., 2008, PLoS One.3:e3858; Pi et al., 2005, J. Biol. Chem. 280:40201-40209; Pi et al., 2010, J. Biol. Chem. 285:39953-39964).

Embryonic development is affected by a variety of environmental signals. In particular, both clinical outcome studies and experimental evidence gathered in model organisms concur to indicate that the mother's health during pregnancy is an important determinant of embryonic development (Osorio et al., 2012, Nature Rev. Endocrinol. 8:624; Lawlor et al., 2012, Nature Rev. Endocrinol. 8:679-688; Challis et al., 2012, Nature Rev. Endocrinol. 8:629-630). By definition, any direct maternal influence on vertebrate embryonic development occurs through the placenta, an organ allowing the transfer of circulating molecules from the mother to the embryo. To date however, molecules either made in the placenta or by the mother, crossing the placenta and that would affect development of the brain of the pup, have not been identified. This is an important question considering that a growing number of epidemiological studies suggest that maternal health may also be a risk factor for neurologic and psychiatric diseases in the offspring (Wadhwa et al., 2001, Prog. Brain Res. 133:131-142; Van den Bergh et al., 2005, Neurosci. Biobehavioral Rev. 29:237-258; Weinstock, 2008, Neurosci. Biobehavioral Rev. 32:1073-1086).

### SUMMARY OF EXEMPLARY EMBODIMENTS

The present disclosure provides exemplary embodiments of methods of treating or preventing cognitive disorders in mammals comprising administering to a mammal in need of treatment for, or prevention of, a cognitive disorder a pharmaceutical composition comprising a therapeutically effective amount of an agent that activates GPR158, the osteocalcin receptor in the brain. In certain exemplary exemplary embodiments, the agent is undercarboxylated/uncarboxylated osteocalcin and the pharmaceutical composition comprises undercarboxylated/uncarboxylated osteocalcin and a pharmaceutically acceptable carrier or excipient. In certain exemplary embodiments, the mammal is a human and the osteocalcin is human osteocalcin. In other exemplary embodiments, the pharmaceutical composition comprises an agent that is not undercarboxylated/uncarboxylated osteocalcin and a pharmaceutically acceptable carrier or excipient. In certain exemplary embodiments, the cognitive disorder is selected from the group consisting of cognitive loss due to neurodegeneration associated with aging, anxiety, depression, memory loss, learning difficulties, and cognitive disorders associated with food deprivation during pregnancy. In certain exemplary embodiments, the cognitive disorder is anxiety due to aging, depression due to aging, memory loss due to aging, or learning difficulties due to aging.

The present disclosure thus provides methods of treating cognitive disorders in mammals comprising administering to a mammal in need of treatment for, or prevention of, a cognitive disorder a pharmaceutical composition comprising an agent that activates GPR158 in an amount that produces an effect in a mammal selected from the group consisting of lessening of cognitive loss due to neurodegeneration associated with aging, lessening of anxiety, lessening of depression, lessening of memory loss, learning difficulties, and lessening of cognitive disorders associated with food deprivation during pregnancy.

In certain exemplary embodiments, the mammal is a human.

In certain exemplary embodiments, the agent is undercarboxylated/uncarboxylated osteocalcin. In certain exemplary embodiments, the agent is human undercarboxylated/uncarboxylated osteocalcin.

In certain exemplary embodiments, the agent is not undercarboxylated/uncarboxylated osteocalcin.

In certain exemplary embodiments, the agent is selected from the group consisting of a small molecule, a peptide, an antibody, or a nucleic acid.

In certain exemplary embodiments where the agent is undercarboxylated/uncarboxylated osteocalcin, at least one of the glutamic acids in the undercarboxylated/uncarboxylated osteocalcin at the positions corresponding to positions 17, 21 and 24 of mature human osteocalcin is not carboxylated. In certain exemplary embodiments, all three of the glutamic acids in the undercarboxylated/uncarboxylated osteocalcin at the positions corresponding to positions 17, 21 and 24 of mature human osteocalcin are not carboxylated.

In certain exemplary embodiments, the undercarboxylated/uncarboxylated osteocalcin is a preparation of undercarboxylated/uncarboxylated osteocalcin in which more than about 20% of the total Glu residues at the positions corresponding to positions 17, 21 and 24 of mature human osteocalcin in the preparation are not carboxylated. In certain exemplary embodiments, the undercarboxylated/uncarboxylated osteocalcin shares at least 80% amino acid sequence identity with mature human osteocalcin when the undercarboxylated/uncarboxylated osteocalcin and mature human osteocalcin are aligned for maximum sequence homology. In certain exemplary embodiments, the undercarboxylated/uncarboxylated osteocalcin shares about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98% amino acid sequence identity with mature human osteocalcin when the undercarboxylated/uncarboxylated osteocalcin and mature human osteocalcin are aligned for maximum sequence homology. In certain exemplary embodiments, the undercarboxylated/uncarboxylated osteocalcin differs at 1, 2, 3, 4, 5, 6, 7, 8, 9, or 10 amino acid residues from mature human osteocalcin.

In certain exemplary embodiments, at least one of the glutamic acids in the undercarboxylated/uncarboxylated osteocalcin at the positions corresponding to positions 17, 21 and 24 of mature human osteocalcin is not carboxylated. In certain exemplary embodiments, all three of the glutamic acids in the undercarboxylated/uncarboxylated osteocalcin at the positions corresponding to positions 17, 21 and 24 of mature human osteocalcin are not carboxylated.

In certain exemplary embodiments, the undercarboxylated/uncarboxylated osteocalcin is a polypeptide selected from the group consisting of:
(a) a fragment comprising mature human osteocalcin missing the last 10 amino acids from the C-terminal end;
(b) a fragment comprising mature human osteocalcin missing the first 10 amino acids from the N-terminal end;
(c) a fragment comprising amino acids 62-90 of SEQ ID NO:2;
(d) a fragment comprising amino acids 1-36 of mature human osteocalcin;
(e) a fragment comprising amino acids 13-26 of mature human osteocalcin;
(f) a fragment comprising amino acids 13-46 of mature human osteocalcin; and
(g) variants of the above.

In certain exemplary embodiments, the pharmaceutical composition comprises an antibody or antibody fragment that binds to and activates GPR158. Preferably, the antibody or antibody fragment is a monoclonal antibody. In certain exemplary embodiments, the antibody or antibody fragment binds to the extracellular domain of GPR158.

In certain exemplary embodiments, the pharmaceutical composition comprises a nucleic acid that activates GPR158. In certain exemplary embodiments, the nucleic acid is an antisense oligonucleotide or a small interfering RNA (siRNA) that decreases expression of β-arrestin.

In certain exemplary embodiments, the pharmaceutical composition comprises about 0.5 mg to about 5 g, about 1 mg to about 1 g, about 5 mg to about 750 mg, about 10 mg to about 500 mg, about 20 mg to about 250 mg, or about 25 mg to about 200 mg, of the agent. In certain exemplary embodiments, the pharmaceutical composition comprises an agent that is formulated into a controlled release preparation. In certain exemplary embodiments, the pharmaceutical composition comprises an agent that is chemically modified to prolong its half life in the human body.

In certain exemplary embodiments, the pharmaceutical composition for treating a cognitive disorder in mammals comprises an undercarboxylated/uncarboxylated osteocalcin polypeptide comprising an amino acid sequence

(SEQ ID NO: 10)
YL YQWLGAPVPYPDPLX₁PRRX₂ VCX₃LNPDCDELADHIGFQEAYR
RFYGPV

wherein

X₁, X₂ and X₃ are each independently selected from an amino acid or amino acid analog, with the proviso that if X₁, X₂ and X₃ are each glutamic acid, then X₁ is not carboxylated, or less than 50 percent of X₂ is carboxylated, and/or less than 50 percent of X₃ is carboxylated,

or said osteocalcin polypeptide comprises an amino acid sequence that is different from SEQ ID NO:10 at 1 to 7 positions other than X₁, X₂ and X₃; and/or wherein the amino acid sequence can include one or more amide backbone substitutions.

In certain exemplary embodiments, the osteocalcin polypeptide of SEQ ID NO:10 is a fusion protein. In certain exemplary embodiments, the arginine at position 43 of SEQ ID NO:10 is replaced with an amino acid or amino acid analog that reduces susceptibility of the osteocalcin polypeptide to proteolytic degradation. In certain exemplary embodiments, the arginine at position 44 of SEQ ID NO:10 is replaced with □-dimethyl-arginine. In certain exemplary embodiments, the osteocalcin polypeptide is a retroenantiomer of uncarboxylated human osteocalcin (1-49).

In certain exemplary embodiments, the patient has or is at risk for a cognitive disorder selected from the group consisting of cognitive loss due to neurodegeneration associated with aging, anxiety, depression, memory loss, learning difficulties, and cognitive disorders associated with food deprivation during pregnancy.

In certain exemplary embodiments of the use described above, the agent that activates GPR158 is undercarboxylated/uncarboxylated osteocalcin. Thus, the present disclosure provides undercarboxylated/uncarboxylated osteocalcin for use in the treatment or prevention of a cognitive disorder in mammals. In particular exemplary embodiments, the cognitive disorder is selected from the group consisting of cognitive loss due to neurodegeneration associated with aging, anxiety, depression, memory loss, learning difficulties, and cognitive disorders associated with food deprivation during pregnancy. In certain exemplary embodiments, the cognitive disorder is anxiety due to aging, depression due to aging, memory loss due to aging, or learning difficulties due to aging.

In certain exemplary embodiments of the use described above, the undercarboxylated/uncarboxylated osteocalcin lessens cognitive loss due to neurodegeneration associated with aging, lessens anxiety, lessens depression, lessens memory loss, improves learning, or lessens cognitive disorders associated with food deprivation during pregnancy. In certain exemplary embodiments, at least one of the glutamic acids in the undercarboxylated/uncarboxylated osteocalcin at the positions corresponding to positions 17, 21 and 24 of mature human osteocalcin is not carboxylated. In certain exemplary embodiments, all three of the glutamic acids in the undercarboxylated/uncarboxylated osteocalcin at the positions corresponding to positions 17, 21 and 24 of mature human osteocalcin are not carboxylated. In certain exemplary embodiments, the undercarboxylated/uncarboxylated osteocalcin is a preparation of undercarboxylated/uncarboxylated osteocalcin in which more than about 20% of the total Glu residues at the positions corresponding to positions 17, 21 and 24 of mature human osteocalcin in the preparation are not carboxylated. In certain exemplary embodiments, the undercarboxylated/uncarboxylated osteocalcin shares about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, or about 98% amino acid sequence identity with mature human osteocalcin when the undercarboxylated/uncarboxylated osteocalcin and mature human osteocalcin are aligned for maximum sequence homology. In certain exemplary embodiments, the undercarboxylated/uncarboxylated osteocalcin differs at 1, 2, 3, 4, 5, 6, 7, 8, 9, or 10 amino acid residues from mature human osteocalcin.

In certain exemplary embodiments of the use described above, the agent is selected from the group consisting of a small molecule, an antibody, or a nucleic acid.

The present disclosure provides the use of an undercarboxylated/uncarboxylated osteocalcin polypeptide, or mimetic thereof, for the manufacture of a medicament for treatment of a cognitive disorder in mammals. In certain exemplary embodiments, the disorder is selected from the group consisting of cognitive loss due to neurodegeneration associated with aging, anxiety, depression, memory loss, learning difficulties, and cognitive disorders associated with food deprivation during pregnancy.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Osteocalcin affects the biosynthesis of neurotransmitters. (Panel A-B) Measure of (Panel A) total activity (XTOT) and (Panel B) ambulatory activity (AMBX), in Osteocalcin−/− (n=6) and Gprc6a−/− (n=6) mice, during 12 hr light and dark phases over a period of three days. Mutant mice were compared to their respective WT (n=6) littermates. (Panel C) Video tracking of an open field paradigm test performed in Osteocalcin−/−, Gprc6a−/− and WT littermate mice. (Panel D-G) HPLC analysis of (Panel D) serotonin, (Panel E) GABA, (Panel F) dopamine, and (Panel G) norepinephrine contents in various parts of Osteocalcin−/− (n=15) and controls (n=15) brains. (Panel H) Quantitative PCR analysis of Tryptophan hydroxylase-2 (Tph2), Glutamate decarboxylase-1 (GAD1), Glutamate decarboxylase-2 (GAD2), Tyrosine hydroxylase (Th) and Aromatic L-amino acid decarboxylase (Ddc) expression levels in the brainstem and midbrain of Osteocalcin−/− (n=13), Gprc6a−/− (n=5) and control (n=11) mice. Error bars represent SEM. Student's T-test is represented on the top of the bars.

FIG. 2. Osteocalcin affects anxiety, depression, memory, and learning. (Panel A-L) Behavioral analysis of (Panel A, C, E, G, I and K) Osteocalcin−/− (n=21), (Panel B, D, F, H, J and L) Gprc6a−/− (n=16) and WT (n=21 and n=15) littermate mice. (Panel A-B) Light and Dark test (L/DT): The latency (Sec=seconds) to enter in the lit compartment, number of transitions between compartments, and amount of time spent in the lit compartment were measured. (Panel C-D) Elevated Plus Maze test (EPMT): Number of entries and amount of time spent (Sec=seconds) in the open arms were scored. (Panel E-F) Open field test (OFT): Total distance (cm), % of the distance traveled, and time spent in the center versus periphery as well as number of rearing events were measured. The video tracking of each group of mice are represented on the right panel. (Panel G-J) Representation of the time spent (seconds) immobile during the (Panel G-H) forced swim test and the (Panel I-J) Tail suspension test. Both tests assess depression-like behavior. (Panel K-L) Morris Water Maze test performed over 10 days. The graphic shows the time (seconds) needed for each group of mice to locate a submerged platform in the swimming area. The video trackings on the left panel are the representations of the standards obtained for each group analyzed. Error bars represent SEM. Student's T-test is represented on the top of the bars.

FIG. 3. Osteocalcin binds to neurons in the brain. (Panel A) Measurement of the total osteocalcin in 3 month-old Osteocalcin−/− mice infused subcutaneously for 7 days with either uncarboxylated osteocalcin (300 ng/hour, right panel) or PBS (left panel). Osteocalcin levels were measured in bone, serum, and different parts of the brain (cortex, midbrain, hypothalamus, brainstem, and cerebellum). (Panel B) Subcutaneous infusion of leptin (50 ng/ml, right panel) or PBS (left panel) for 7 days in ob/ob mice. Leptin levels were measured in serum, cortex, midbrain, hypothalamus, brainstem, and cerebellum. (Panel C) Binding of GST-biotin (30 □g/ml) (panel 1) and biotinylated osteocalcin (300 ng/ml) (panels 2-4) to the dorsal (DR) and median (MR) raphe nuclei of the brainstem (identified by anti-5-HT immunofluorescence), to the ventral tegmental area (VTA) of the midbrain (identified by anti-TH immunofluorescence), and to the CA3 and CA4 of the hippocampus (identified anatomically). Panel 5 shows competition with unlabeled osteocalcin (1,000-fold excess). Binding with GST-biotin, osteocalcin-biotinylated, and competition assays were performed on adjacent sections. (Panel D) Expression of Tph2 and GAD 1 in brainstem, and Th in midbrain explants from WT and Gprc6a−/− mice, treated with 10 ng/ml osteocalcin or vehicle. (Panel E) Gene expression in WT primary hindbrain neuron cultures treated with 10 ng/ml osteocalcin or vehicle. (Panel F) Calcium flux response of primary hindbrain cultured neurons to osteocalcin treatment. (Panel G-H) Extracellular current recordings of (Panel G) neurons of the dorsal raphe nucleus and (Panel H) GABAergic interneurons of the brainstem treated with osteocalcin (10 ng/ml).

FIG. 4. Administration of osteocalcin prevents anxiety and depression. (Panel A-E) Behavioral analyses of adult Osteocalcin−/− mice receiving osteocalcin through intracerebro-ventricular (ICV) infusions. (Panel A) Light and Dark test, (Panel B) Elevated plus maze test, (Panel C) Open field test, (Panel D) Forced swim test, and (Panel E) Tail suspension test performed in a cohort of WT (n=7) and Osteocalcin−/− infused with vehicle or osteocalcin (10 ng/hour). In each set of three bars, the rightmost bar represents the results following administration of osteocalcin.

FIG. 5. (Panel A) Expression of osteocalcin in the brains of WT mice is not detected above that in the brains of Osteocalcin−/− mice as judged by quantitative PCR. (Panel B) Expression of osteocalcin in the brains of WT mice is not detected above that in the brains of Osteocalcin−/− mice as judged by in situ hybridization. (Panel C) m-Cherry expression is seen in bone but not in the brain of a mouse model in which the m-Cherry gene was knocked into the Osteocalcin locus. (Panel D) Tamoxifen-treated Osteocalcinosbert2−/− mice showed a significant increase in anxiety-like and depression-like behavior when compared to α1(I)Collagen-Creert2 or Osteocalcinflox/flox mice as judged by the DLT test. (Panel E) Tamoxifen-treated Osteocalcinosbert2−/− mice showed a significant increase in anxiety-like and depression-like behavior when compared to α1(I)Collagen-Creert2 or Osteocalcinflox/flox mice as judged by the EPM test. (Panel F) Tamoxifen-treated Osteocalcinosbert2−/− mice showed a significant increase in anxiety-like and depression-like behavior when compared to α1(I)Collagen-Creert2 or Osteocalcinflox/flox mice as judged by the tail suspension test. (Panel G) Tamoxifen-treated Osteocalcinosbert2−/− mice showed a significant increase in anxiety-like and depression-like behavior when compared to α1(I)Collagen-Creert2 or Osteocalcinflox/flox mice as judged by the tail suspension test. (Panel H) Tamoxifen-treated Osteocalcinosbert2−/− mice showed a significant increase in anxiety-like and depression-like behaviors when compared to α1(I)Collagen-Creert2 or Osteocalcinflox/flox mice as judged by the EPM test. (Panel I) Spatial learning and memory are affected in tamoxifen-treated Osteocalcinosbert2−/− mice.

FIG. 6. Maternal osteocalcin favors fetal neurogenesis. (Panel A) Expression of osteocalcin (qPCR) in bone, brain, and placenta of WT and Ocn−/− newborns (postnatal day [P] 0) and embryos (E13.5-E18.5). (Panel B) Osteocalcin circulating levels in WT or Ocn−/− newborns (P0) and embryos (E13.5-E18.5). (Panel C) Ex vivo dual-perfusion system that monitors the transport of osteocalcin across the placenta. Uncarboxylated mouse osteocalcin (300 ng/ml) was injected through the uterine artery in placentas obtained from WT mice at E14.5, E15.5, and E18.5 of pregnancy. Osteocalcin in fetal eluates is represented as % of maternal input. (Panel D) Circulating levels of osteocalcin in WT embryos originating from WT or Ocn+/− mothers, of Ocn+/− embryos originating from Ocn+/− or Ocn−/− mothers, and of Ocn−/− embryos originating from Ocn+/− or Ocn−/− mothers. Measurements were performed at E16.5 and E18.5. (Panel E) Cresyl violet stain of lateral ventricles of hippocampi of E18.5 WT embryos originating from WT mothers and Ocn−/− embryos originating from Ocn+/− or Ocn−/− mothers. The measurements of the lateral ventricle area over brain area are represented below the images (in %) (scale bars=0.5 mm). (Panel F) Number of apoptotic cells (stained by TUNEL assay) in hippocampi of E18.5 WT embryos carried by WT mothers and Ocn−/− embryos carried by Ocn+/− or Ocn−/− mothers. (Panel G and H) CFC (Panel G) and NOR (Panel H) performed inWT and Ocn−/− mice born from Ocn−/− or Ocn+/− mothers (n=7-18 per group). In the CFC, Ocn−/− mice born from Ocn−/− mother mice exhibited significantly less context-elicited freezing than WT mice in context A and A′. In the NOR, there was a significant increase in the exploratory period in Ocn−/− mice born from Ocn−/− mothers compared to Ocn−/− mice born from Ocn+/− mothers or WT mice when a novel object was introduced. (Panel I and J) BrdU and DCX Immunohistochemistry showing a significantly lower number of BrdU+(Panel I) and DCX+(Panel J) cells in the dentate gyms (DG) of WT and Ocn−/− mice born from Ocn−/− or Ocn+/− mothers. This decrease was even more pronounced in the ventral region of the DG. (Scale bars=0.2 mm.) For (Panel A)-(Panel F), (Panel I), and (Panel J), the statistical test on the top of each graph represents the Student's t test; p<0.05 is significant. For (Panel G) and (Panel H), the statistical test on the top of each graph represents an ANOVA. Significant ANOVAs were followed up with Fisher's PLSD tests where appropriate. *p value<0.05, **p value<0.01, ***p value<0.001.

FIG. 7. Maternal osteocalcin determines spatial learning and memory in adult offspring. (Panel A-F) DLT (Panel A), EPMT (Panel B), OFT (Panel C), FST (Panel D), TST (Panel E), and MWMT (Panel F) performed in 3-month-old Ocn−/− mice born from Ocn−/− mothers injected once a day with vehicle or osteocalcin (240 ng/day) during pregnancy compared to WT mice. (Panel G) Surface of the lateral ventricle over brain area (%) of E18.5 hippocampi coronal sections of WT embryos originating from WT mothers and Ocn−/− embryos originating from osteocalcin-injected Ocn−/− mothers. (Panel H) Number of apoptotic cells (stained by TUNEL assay) of E18.5 hippocampi coronal sections of WT embryos originating from WT mothers and Ocn−/− embryos originating from Ocn−/− mothers injected with osteocalcin (240 ng/day). (Panel I) Cresyl violet, NeuN immunofluorescence, and dentate gyms area (% versus WT) of WT and Ocn−/− embryos originating from osteocalcin-injected Ocn−/− mothers. Scale bars=0.5 mm. (Panel J) Serotonin content in the hippocampus of Osteocalcin−/− E18.5 embryos originating from injected Osteocalcin−/− mothers compared to the ones originating from uninjected Osteocalcin−/− mothers. (Panel K) GABA content in the hippocampus of Osteocalcin−/− E18.5 embryos originating from injected Osteocalcin−/− mothers compared to the ones originating from uninjected Osteocalcin−/− mothers.

FIG. 8. Osteocalcin improves cognitive function in adult wild-type (WT) mice. Results from dark and light (DLT) and elevated plus maze tests (EPMT) performed in 3-month old WT mice infused ICV with vehicle (PBS) or Ocn (3, 10, 30 ng/hour) are shown. (Panel A) DLT measuring the latency to enter, the number of entries, and the time spend in lit compartment. (Panel B) EPMT measuring the number of entries into open arms and the time spend in lit compartments.

FIG. 9. Osteocalcin improves hippocampal function in aged wild-type (WT) mice. Constant and novel object investigation in the Novel Object Recognition test in 17 month old mice treated for 1 month with vehicle or 10 ng/hr recombinant uncarboxylated osteocalcin.

FIG. 10. Osteocalcin administration results in CREB phosphorylation. (Panel A) p-CREB immunofluorescence (IF) in the dentate gyms (DG) of WT and Ocn−/− hippocampal region. (Panel B) p-CREB IF in WT brain sections following a dual stereotactic injection of vehicle (PBS) (on the left) or Ocn (10 ng) (on the right) in the hippocampus. The arrows point toward the DG. (Panel C) PKA IF in WT brain sections following a dual stereotactic injection of vehicle (PBS) (on the right) or Ocn (10 ng) (on the left) in the hippocampus.

FIG. 11. CREB activation by osteocalcin is functionally relevant. Contextual fear conditioning in 3.5 month old mice injected acutely with 10 ng recombinant uncarboxylated osteocalcin 24 hours prior to context exposure. 3 shocks of 0.55 mA were delivered to mice 1 min apart. On Day 1, % freezing was the same for both groups. % freezing was measured again 24 hours after the initial shocks. Osteocalcin injected mice showed increased freezing along with hyperexcitability.

FIG. 12. Influence in bone health on cognition through osteocalcin. (Panel a) Runx2 accumulation (Western blot) in various tissues of 3 month-old WT mouse. Gapdh was used as a loading control. (Panel b) Circulating levels of bioactive osteocalcin in 3 month-old Runx2 and WT littermates.(Panel c) Circulating levels of bioactive osteocalcin in 3 month-old Ocn+/− and WT littermates. (Panel d) Glutamate decarboxylase-1 (Gad 1), and Tyrosine hydroxylase (Th) expression (qPCR) in the brainstem and midbrain of 3 month-old Runx2+/− and WT littermates. (Panel e) Brain-derived neurotrophic factor (BDNF) accumulation (representative Western blot, left) and quantification of band intensities (right) in hippocampi of 3 month-old Runx2+/− and WT littermates. β-tubulin is used as a loading control. (Panel f) Dark to Light Transition (DLT) test performed in Runx2+/− and WT littermates. Time spent in the lit compartment and open arms was measured. (Panel g) Dark to Light Transition (DLT) test performed in Ocn+/− and WT littermates. Time spent in the lit compartment and open arms was measured. (Panel h) Elevated Plus Maze (EPMT) test performed in Runx2+/− and WT littermates. Number of entries and time spent (s) in the open arms were scored. (Panel i) Elevated Plus Maze (EPMT) test performed in Ocn+/− and WT littermates. Number of entries and time spent (s) in the open arms were scored. (Panel j) Morris water maze test (MWMT) performed over 10 days. The graphic shows the time (s) needed for each group of mice, Runx2+/− and WT littermates, to localize a submerged platform in the swimming area. (Panel k) Novel object recognition (NOR) performed in Runx2+/− and WT littermates. Preference index (time spent with novel object/total exploration time) was measured. (Panel 1) MWMT performed over 10 days. The graphic shows the time (s) needed for each group of WT mice, either vehicle-treated, alendronate-treated, or alendronate+osteocalcin-treated, to localize a submerged platform in the swimming area. (Panel m) NOR performed in vehicle-treated, alendronate-treated, and alendronate+osteocalcin-treated WT mice. Preference index (time spent with novel object/total exploration time) was measured. Results are given as mean±s.e.m. *P≤0.05 **P≤0.01 ***P≤0.001, n.s., not significant; by Student's t-test compared to vehicle or WT (b-i, k), or by two-way repeated measures ANOVA followed by Fisher's LSD test (j−1).

FIG. 13. Exogenous osteocalcin improves anxiety and cognition in aged WT mice. (Panel a) EPMT performed in aged mice receiving plasma from aged, young WT, or young Ocn−/− mice, and aged WT mice receiving plasma from young Ocn−/− supplemented with 90 ng/g osteocalcin. Number of entries and time spent (s) in the open arms were scored. (Panel b) NOR performed in aged WT mice receiving plasma from WT mice either aged or young, or from young Ocn−/− mice or from young Ocn−/− supplemented with 90 ng/g osteocalcin. Preference index (time spent with novel object/total exploration time) was measured for each group. (Panel c) BDNF accumulation (representative Western blot, left) and quantification of band intensities (right) in hippocampi of aged WT mice receiving plasma from WT, either aged or young, or from young Ocn−/− mice. α-Tubulin is used as a loading control. (Panel d) DLT performed in 12 and 16 month-old WT mice treated with vehicle or osteocalcin. Number of entries in the lit compartment was measured. (Panel e) EPMT performed in 12 and 16 month-old WT mice treated with vehicle or osteocalcin. Time spent in the lit compartment and open arms was measured. (Panel f) MWMT performed over 10 days in 12 and 16 month-old WT mice treated with vehicle or osteocalcin. The graph shows the time to localize a submerged platform in the swimming area. (Panel g) NOR performed in 12 and 16 month old WT mice treated with vehicle or osteocalcin. Preference index (time spent with novel object/total exploration time) was measured for each group. (Panel h) BDNF accumulation (Western blot) in the hippocampus of WT mice injected peripherally with vehicle, kainic acid used as a positive control, or osteocalcin for 16 hours. β-actin is used as a loading control. Results are given as mean±s.e.m. *P≤0.05 **P≤0.01 ***P≤0.001, n.s., not significant; by Student's t-test compared to vehicle (d-e, g-h); by one-way ANOVA followed by Fisher's LSD test (a-c); or by two-way repeated measures ANOVA followed by Fisher's LSD test (f).

FIG. 14. Identification of the putative receptor of Osteocalcin in the hippocampus and midbrain. (Panel a) In situ hybridization of Gpr158 in E14.5 WT embryos. (Panel b) In situ hybridization of Gpr158, Gpr156, Gpr179, Gprc5a, Gprc5b, Gprc5c and Gprc5b in the brain of 10 day-old WT mice. (Panel c) In situ hybridization of Gpr158 in the brain of 3 month-old WT mice. For the VTA, Th was used as a positive control. (Panel d) Immunofluorescence of Gpr158, Map2 and Gfap in primary hippocampal neurons (DIV 15). (Panel e) Expression of Gpr158 in tissues of 3 month-old WT mice. Expression Gpr158 was compared to the one in cerebellum. (Panel f) Pull-down assay using biotinylated-osteocalcin on solubilized Ocn−/− hippocampal membrane. Purified proteins were subjected to a Western Blot using anti-Gpr158 and anti-G□q. (Panel g) Gpr158 accumulation (Western blot, left) and quantification of band intensities (right) in from solubilized membrane from WT or Ocn−/− hippocampi. Na,K ATPase is used as a loading control. Results are given as mean±s.e.m. *P≤0.05 by Student's t-test compared to WT (g)

FIG. 15. Function analysis of Osteocalcin signaling through Gpr158. (Panel a) IP1 accumulation in WT and Gpr158−/− hippocampal neurons (DIV 15) treated with either vehicle or osteocalcin for 1 hour. Glutamate was used as a positive control. (Panel b) Expression (qPCR) of Th and Bdnf in the midbrain of 6 month-old Gpr158+/− and WT littermates. (Panel c) Expression (qPCR) of Bdnf in WT and Gpr158−/− hippocampal neurons (DIV 15) treated with either vehicle or osteocalcin for 4 hours. (Panel d) Osteocalcin's effect on spontaneous action potential (AP) frequency in CA3 pyramidal neurons in WT (4 of 4 cells) and Gpr158−/− mice. The bars above the recording traces indicate the application of osteocalcin. (Panel e) EPMT performed in 3 month-old Gpr158−/−, Gpr158+/−, and WT littermates. Number of entries and time spent (s) in the open arms were scored. (Panel f) DLT performed in 3 month-old Gpr158−/−, Gpr158+/−, and WT littermates. Time spent in the lit compartment and open arms was measured. (Panel g) Open field test performed in 3 month-old Gpr158−/−, Gpr158+/−, and WT littermates. Total ambulation (cm) and time spent in the center of the arena (s) were measured. (Panel h) MWMT performed over 10 days in 3 month-old Gpr158−/− and WT littermates. The graph shows the time (s) to localize a submerged platform in the swimming area. (Panel i) NOR performed in 3 month-old Gpr158−/−, Gpr158+/−, Ocn+/−, Gpr158+/−; Ocn+/− and WT littermates. Preference index (time spent with novel object/total exploration time) was measured. (Panel j) NOR performed in 3 month-old sh-control- or sh-Gpr158-injected mice. After recovery, mice were injected with saline or osteocalcin (10 ng). Preference index (time spent with novel object/total exploration time) was measured. (Panel k) CFC performed in 3 month-old sh-control- or sh-Gpr158-injected mice. After recovery, mice were injected with saline or osteocalcin (10 ng). Percent freezing 24 hours after training was measured. Results are given as mean±s.e.m. *P≤0.05 **P≤0.01 ***P≤0.001, n.s.: not significant; by Student's t-test compared to WT or untreated (Panel a-c); by one-way ANOVA followed by Fisher's LSD test (Panel e-g, i); or by two-way repeated measures ANOVA followed by Fisher's LSD test (Panel h, j-k).

FIG. 16. Amino acid sequence encoding human GPR158 from NCBI reference sequence NP 065803.2 (SEQ ID NO: 6).

FIG. 17A-C. Nucleotide sequence encoding human GPR158 from NCBI reference sequence NM 020752.2 (SEQ ID NO: 7).

FIG. 18. Amino acid sequence encoding human GPR158 from NCBI reference sequence NM 020752.2 (SEQ ID NO: 8).

FIG. 19A-B. Nucleotide sequence encoding human GPRC6A from Genbank Accession No. AF502962 (SEQ ID NO: 11).

FIG. 20. Amino acid sequence encoding human GPRC6A from Genbank Accession No. AF502962 (SEQ ID NO: 12).

Throughout the drawings, the same reference numerals and characters, unless otherwise stated, are used to denote like features, elements, components, or portions of the illustrated embodiments. Similar features may thus be described by the same reference numerals, which indicate to the skilled reader that exchanges of features between different embodiments can be done unless otherwise explicitly stated. Moreover, while the present disclosure will now be described in detail with reference to the figures, it is done so in connection with the illustrative embodiments and is not limited by the particular embodiments illustrated in the figures. It is intended that changes and modifications can be made to the described embodiments without departing from the true scope and spirit of the present disclosure as defined by the appended claims.

### DETAILED DESCRIPTION OF EXEMPLARY EMBODIMENTS

The exemplary embodiments present disclosure is based in part on the discovery of a previously unknown biochemical pathway linking osteocalcin and cognitive processes in mammals. The present inventors have discovered that osteocalcin crosses the blood-brain barrier, binds to GPR158 and signals in neurons of the brainstem, inhibits GABA, and favors serotonin and dopamine synthesis by increasing the activity of enzymes involved in the synthesis of serotonin and dopamine. These effects lead to beneficial effects on cognitive functions such as memory, learning, anxiety, and depression, as well as to beneficial effects on neurodegeneration associated with aging.

Using mouse models with decreased bone formation or bone resorption, it is shown here that bone health is a significant determinant of anxiety and cognition, in part through osteocalcin, and that osteocalcin is necessary and sufficient to correct the anxiety and cognitive decline that develops with aging. To begin deciphering how osteocalcin transduces its signal in neurons, expression, biochemical, stereotaxic lentiviral-based gene downregulation, and cell-based and genetic assays were used. These led to the identification of an orphan GPCR that is expressed in the midbrain and hippocampus, GPR158, as being necessary to mediate osteocalcin's regulation of anxiety and memory in an inositol triphosphate (IP3)-dependent manner. These results reveal an unanticipated ability of skeletal health to reduce anxiety and improve memory and identify molecular tools that could be used to harness this pathway for therapeutic purposes in the aging population.

The multiple links that tie the production of bioactive osteocalcin and bone remodeling together pose the question of to what extent bone health influences anxiety and cognitive functions. Given where osteocalcin is synthesized and how it becomes active as a hormone (Karsenty, G. & Ferron, M., 2012, Nature 481:314-320), this question was addressed by testing the impact of either arm of bone remodeling, formation and resorption, on anxiety and cognition.

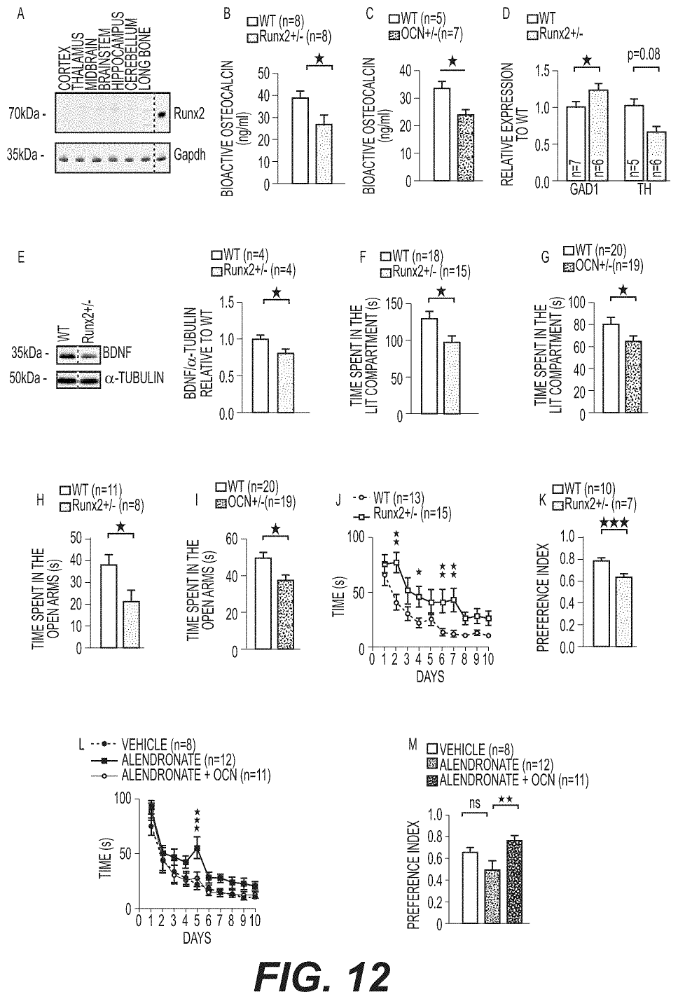

Osteocalcin is synthesized by osteoblasts, the bone-forming cells (Ferron, M. et al., 2010, Cell 142:296-308). To determine the influence of bone formation on anxiety and cognition, mice lacking one allele of Runx2, a master regulator of osteoblast differentiation (Ducy, P., et al., 1997, Cell 89:747-754) that is not detected in the brain, were studied because Runx2+/− mice display reduced bone formation (Ducy, P., et al., 1997, Cell 89:747-754; Lee, B. et al., 1997, Nat Genet 16:307-310) and a 50% decrease in circulating bioactive osteocalcin levels (FIG. 12a-b). Expression of Gadl, a gene down-regulated by osteocalcin signaling (Oury, F. et al., 2013, Cell 155:228-241), was increased, whereas expression of Th, a gene up-regulated by osteocalcin signaling (Oury, F. et al., 2013, Cell 155:228-241), was decreased in the Runx2+/− brainstem and midbrain (FIG. 12d). Furthermore, accumulation of Bdnf, a marker of hippocampal-dependent memory formation (Anastasia, A. et al., 2013, Nat Commun 4:2490; Hall, J., et al., 2000, Nat Neurosci 3:533-535; Nagahara, A. H. et al., 2009, Nat Med 15:331-337) whose regulation by bone derived signals has not previously been reported (Oury, F. et al., 2013, Cell 155:228-241) was decreased in Runx2+/− hippocampi (FIG. 12d). Anxiety-like and exploratory behaviors were next analyzed in 3 month-old Runx2+/− mice and control littermates. In the dark to light transition test (DLT), which is based on the innate aversion of rodents to brightly illuminated areas and their decreased spontaneous exploratory behavior in response to light (Oury, F. et al., 2013, Cell 155:228-241; David, D. J. et al., 2009, Neuron 62:479-493; Crawley, J. N., 1985, Neurosci Biobehav Rev 9:37-44; Zernig, G., et al., 1992, Neurosci Lett 143:169-172; Vicente, M. A., et al., 2008, Neurosci Lett 445:204-208), Runx2+/− mice spent less time in the lit compartment than WT littermates (FIG. 12f). In the elevated plus maze test (EPMT), anxiety results in a shorter time spent in the open arms (Oury, F. et al., 2013, Cell 155:228-241; Nagahara, A. H. et al., 2009, Nat Med 15:331-337; David, D. J. et al., 2009, Neuron 62:479-493). Again, Runx2+/− mice spent less time in the open arms than WT littermates (FIG. 12h). Spatial learning and memory were also assessed through two tests. In the Morris water maze test (MWMT), Runx2+/− mice showed a significant delay in learning the location of the platform over 10 days compared to WT littermates (FIG. 12j). In the novel object recognition test (NOR) (Oury, F. et al., 2013, Cell 155:228-241; Ennaceur, A. & Delacour, J., 1988, Behav Brain Res 31:47-59; Denny, C. A., et al., 2012, Hippocampus 22:1188-1201) which evaluates hippocampal-dependent memory (Oury, F. et al., 2013, Cell 155:228-241; Denny, C. A., et al., 2012, Hippocampus 22:1188-1201; Broadbent, N. J., et al., 2010, Learn Mem 17:5-11), Runx2+/− mice spent significantly less time exploring the novel object than WT littermates (FIG. 12k). These observations, which are overall similar to those made in Osteocalcin+/− mice (Oury, F. et al., 2013, Cell 155:228-241) (FIG. 12c, g, i), indicate that impairing bone formation increases anxiety and hampers spatial learning and memory. Of note, cognitive defects have been reported in patients haplo-insufficient for Runx2 (Izumi, K. et al., 2006, Am J Med Genet A 140:398-401; Takenouchi, T., et al., 2014, Eur J Med Genet 57:319-321).

Because osteocalcin becomes active as a hormone after it has become undercarboxylated due to the low pH existing in the resorption lacuna (Ferron, M. et al., 2010, Cell 142:296-308), the influence of bone resorption on anxiety and cognition was examined. A 3 week-long treatment with alendronate, a small molecule inhibitor of bone resorption (Drake, M. T., et al., 2008, Mayo Clin Proc 83:1032-1045), not only inhibited bone resorption but also decreased the circulating levels of bioactive osteocalcin. Alendronate-treated mice displayed a delay in learning in the MWMT and a memory deficit in NOR. Importantly, these behavioral abnormalities were corrected by peripheral delivery of osteocalcin (FIG. 12l-m). Taken together, these experiments indicate that healthy bone remodeling is necessary to reduce anxiety and to enhance cognition, and that these beneficial effects are mediated in part by osteocalcin.

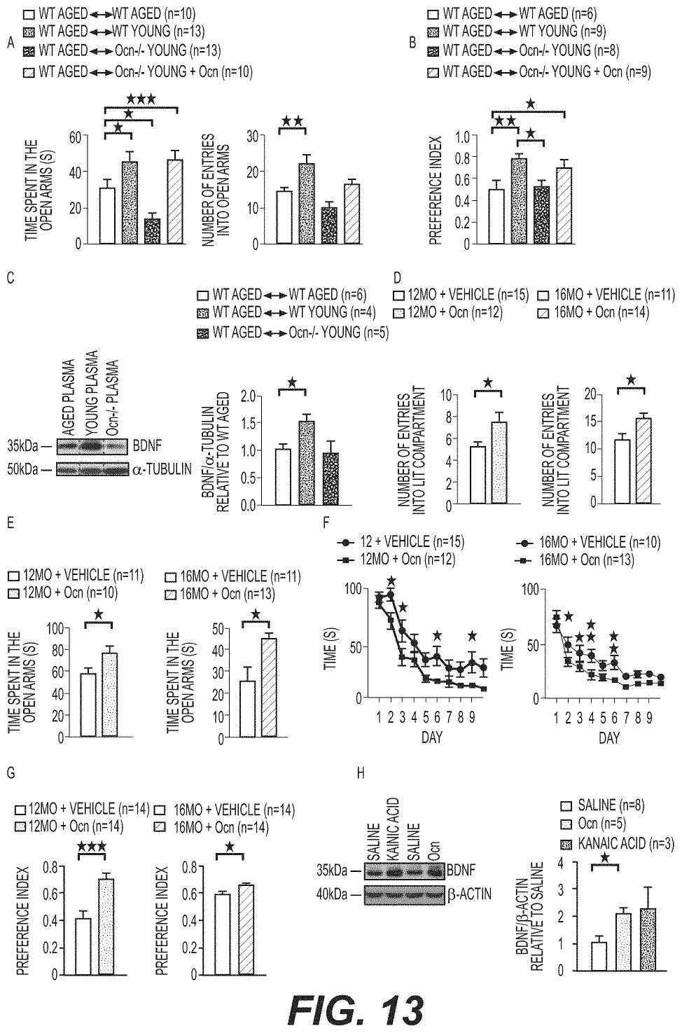

The influence of bone health on anxiety and cognition described above raised the question of whether the decrease in bone health that occurs with age (Ebbesen, E. N., et al., 1999, J Bone Miner Res 14:1394-1403) contributes to age-related decline in cognitive functions. The decrease in circulating osteocalcin levels that occurs around midlife (Mera, P. et al., 2016, Cell Metab 23:1078-1092) raised an even more precise question: to what extent does osteocalcin mediate the influence of bone on cognitive health? To answer this question, whether osteocalcin is necessary for the beneficial effect of plasma from young mice on cognition and anxiety in older mice was investigated. As previously reported, 16 month-old WT mice receiving plasma from 3-month-old WT mice were significantly less anxious and had improved hippocampus-dependent memory compared to those receiving plasma from aged WT mice (Villeda, S. A. et al.,2014, Nat Med 20:659-663) (FIG. 13a-b). Importantly, this improvement was not observed if 16 month-old WT mice instead received plasma obtained from 3-month-old Osteocalcin−/− mice (FIG. 13a-b). That Bdnf accumulation was increased in the hippocampus of 16-month-old WT mice receiving plasma from young WT mice, but not in those receiving plasma from young Osteocalcin−/− or from aged WT mice further suggested that Bdnf is an osteocalcin regulated gene (FIG. 13c). To establish that osteocalcin is necessary to trigger the beneficial effects of plasma from young mice and to rule out any developmental component to the effect of osteocalcin, 16-month-old WT mice were injected with plasma from Osteocalcin−/− mice that had been supplemented with mouse recombinant osteocalcin (90 ng/g). This injection, which increased circulating levels of osteocalcin, resulted in an improvement in anxiety and memory in 16-month-old WT mice comparable to that resulting from the administration of plasma from young WT mice (FIG. 13a-b).

To determine if exogenous osteocalcin would suffice to improve anxiety and cognition in WT mice as they age vehicle or osteocalcin (30 or 90 ng/h) was delivered to 10- or 14-month-old WT mice peripherally for 60 days via mini-pumps prior to analyzing behavior since osteocalcin crosses the blood brain barrier. Whether tested through the DLT or EPMT, 12 and 16 month-old osteocalcin-treated mice showed better exploratory behavior and decreased anxiety-like behavior compared to vehicle-treated littermates (FIG. 13d-e). Likewise, when tested through MWMT and NOR, memory was significantly improved in 12- and 16-month-old osteocalcin-treated mice compared to vehicle-treated littermates (FIG. 13f-g). These experiments demonstrate that when delivered peripherally, exogenous osteocalcin is sufficient to decrease anxiety and improve memory in 12- and 16-month-old WT mice. That Bdnf accumulation were increased in the hippocampi of mice receiving osteocalcin adds further support to the notion that osteocalcin regulates expression of this gene in the hippocampus (FIG. 13h).

By highlighting the importance of osteocalcin in the regulation of anxiety and cognitive functions in aged mice, this body of data raised the question of the signaling pathway used by this hormone in the brain. The bell-shaped curve of osteocalcin signaling in neurons (Oury, F. et al., 2013, Cell 155:228-241) suggested that like Gprc6a, osteocalcin's receptor in peripheral tissues, the receptor of this hormone in the brain might be a GPCR. For this reason, a search was conducted for an orphan GPCR that: (1) like Gprc6a, would belong to the class C family of GPCRs (Chun, L., et al., 2012, Acta Pharmacol Sin 33:312-323); (2) would be expressed in the ventral tegmental area (VTA) of the midbrain and in the hippocampus (Oury, F. et al., 2013, Cell 155:228-241) but 3] would not be expressed in any cell type where Gprc6a is expressed. Analysis of the expression pattern of all orphan class C

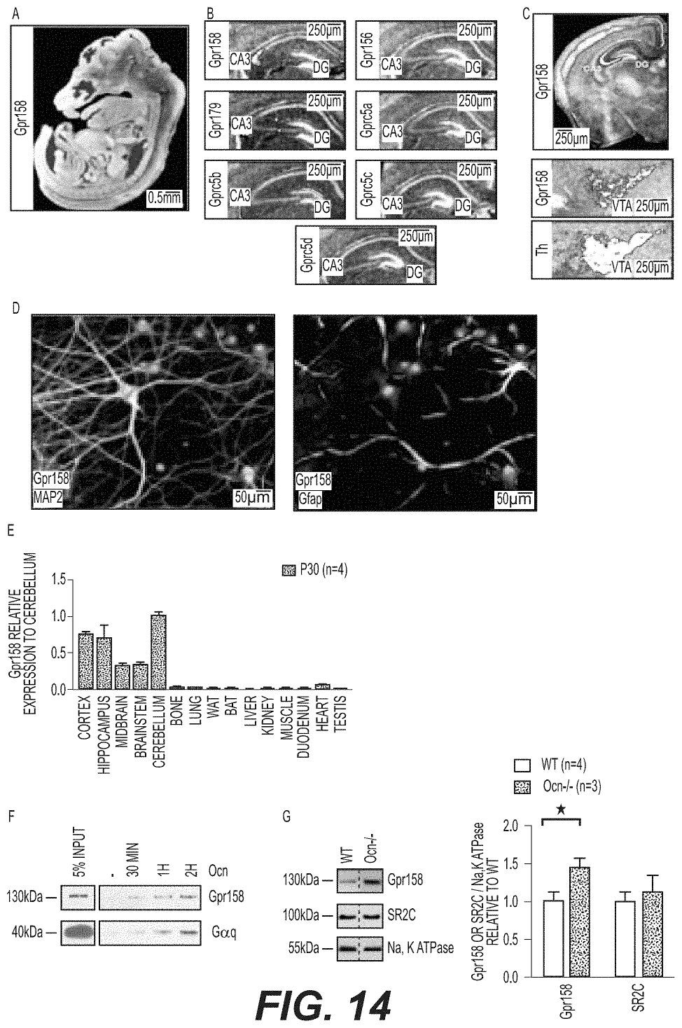

GPCRs identified Gpr158 as being the only one that is expressed in the VTA and in the CA3 region of the hippocampus, where osteocalcin has been previously shown to bind (Oury, F. et al., 2013, Cell 155:228-241) (FIG. 14a,b). GPR158 is also expressed in the somatosensory, motor and auditory area of the cortex, the piriform cortex and the retrosplenial area (FIG. 14c). An immunofluorescence study conducted on primary hippocampal neurons culture showed that Gpr158 is expressed in neurons and not in glial cells (FIG. 14d). Moreover, unlike any other orphan class C Gper, Gpr158 is not expressed in peripheral tissues where osteocalcin signals through Gprc6a, either during development or after birth (FIG. 14a, e). In a pull-down assay performed on solubilized membranes from hippocampal tissue, biotinylated osteocalcin could bind a complex containing Gpr158 and the G□q subunit; additionally, Gpr158 is more abundant in Osteocalcin−/− than in WT hippocampi (FIG. 14f-g). These results support the hypothesis that Gpr158 might be a necessary component of osteocalcin's signaling machinery in the midbrain and hippocampus. Next, biochemical, electrophysiological, and behavioral assays were used on WT and Gpr158−/− cells or mice to determine whether this is the case.

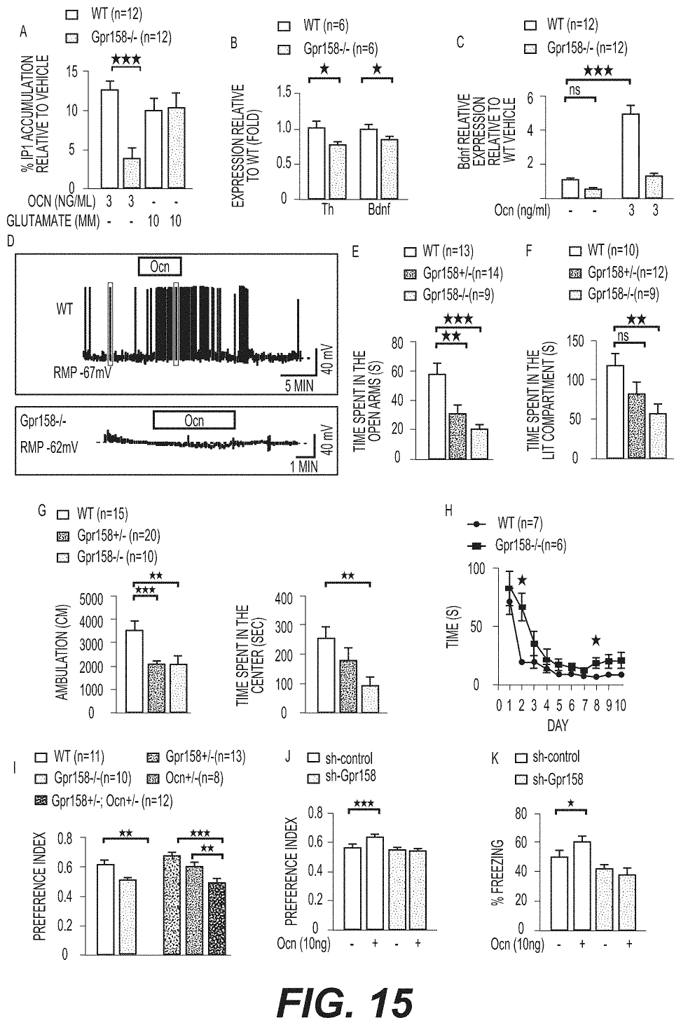

Recombinant osteocalcin did not affect cAMP production, but rather increased the production of IP1, a byproduct of the second messenger IP3, in WT cultured hippocampal neurons; this effect was far less pronounced in Gpr158−/− neurons. Glutamate was used as a positive control in these experiments (FIG. 15a). This result is consistent with the interaction of Gpr158 and Gaq (FIG. 14g). Concordant with the notion that Gpr158 is necessary to transduce osteocalcin signal in the brain, expression of Th and Bdnf, two target genes of osteocalcin, was lower in Gpr158−/− than in WT midbrain, and recombinant uncarboxylated osteocalcin increased Bdnf expression in WT significantly more than in Gpr158−/− hippocampal neurons (FIG. 15b-c). Whole-cell current clamp recording showed that osteocalcin significantly enhanced the action potential frequency in pyramidal cells of the CA3 region of WT but not of Gpr158−/− hippocampi (FIG. 15d). In vivo, when tested in the EPMT and DLT, 3 month-old Gpr158+/− and Gpr158−/− mice were significantly more anxious than WT littermates, as were Osteocalcin+/− and −/− mice (FIG. 15e-f). In a third test, the open field test, anxiety results in a decrease in total ambulation and time spent in the center of the box; these parameters were also significantly decreased in 3 month-old Gpr158+/− and −/− mice as they were in Osteocalcin-deficient mice when compared to WT littermates (FIG. 15g). Spatial learning and memory were assessed through the MWMT and NOR. In both tests, 3 month-old Gpr158−/− mice demonstrated a decrease in learning, although their deficit in the MWMT was less severe than what was observed in Osteocalcin−/− mice (FIG. 15h-i). To determine in vivo whether Gpr158 is a necessary component of the signaling apparatus used by osteocalcin to promote memory, two distinct experiments were performed. First, lentivirus expressing either shRNA targeting Gpr158 (60% decrease in Gpr158 protein levels), or scrambled shRNA as a control, was injected in the anterior hippocampus of WT mice. Fifteen days later, osteocalcin (10 ng) was injected at the same stereotactic coordinates. Osteocalcin enhanced memory performance as assayed by the NOR in control mice but not in mice in which Gpr158 expression had been efficiently downregulated (FIG. 15j). A similar result was obtained when mice were tested for contextual fear conditioning (CFC), a test measuring associative memory that requires the integrity of the hippocampus (FIG. 15k). Second, 3 month-old Gpr158+/−, or Osteocalcin+/− mice and compound heterozygous Gpr158+/−; Osteocalcin+/− mice were subjected to the NOR. While single heterozygous mice did not display any abnormalities in this test, Gpr158+/−; Osteocalcin+/− mice behaved similarly to Gpr158−/− or Osteocalcin−/− mice (FIG. 15i). These results are consistent with the notion that Gpr158 is a necessary component in osteocalcin's regulation of cognitive functions.

The increase in anxiety and the decline in cognition seen in the aging population is a growing public health concern and an unmet medical need. The results presented here identify a hormonal and molecular pathway that is sufficient in the mouse to reduce anxiety and to reverse age-related cognitive decline. In addition to their therapeutic potential, these findings pave the way to elucidate the functions and molecular mechanism of action of osteocalcin in other regions of the brain besides the VTA and the hippocampus where its receptor is expressed.

The exemplary embodiments of the present disclosure is also based in part on the observation that maternally-derived osteocalcin crosses the placenta and prevents neuronal apoptosis in mouse embryos. Uncarboxylated osteocalcin injections in Osteocalcin−/− mouse mothers throughout pregnancy prevent this neuronal apoptosis. These observations indicate that osteocalcin is a critical regulator of neuronal apoptosis and that administration of undercarboxylated/uncarboxylated osteocalcin may be useful in the treatment or prevention of diseases where neuronal apoptosis plays an important role.

Moreover, direct administration of undercarboxylated/uncarboxylated osteocalcin to the brains of adult Osteocalcin−/− mice (mice completely lacking osteocalcin expression) rescued defects in anxiety, depression, learning, and memory in the mice. Since undercarboxylated/uncarboxylated osteocalcin can cross the blood/brain barrier, this result indicates that administration of undercarboxylated/uncarboxylated osteocalcin in such a manner as to increase the blood concentration of undercarboxylated/uncarboxylated osteocalcin in a mammal should provide benficial effects on cognitive functions relating to anxiety, depression, learning, and memory.

In view of the observations described herein, it is concluded that osteocalcin regulates cognitive functions such as anxiety, depression, learning, and memory by binding to and activating GPR158. Thus, certain aspects of the present disclosure are directed to the therapeutic use of agents that activate GPR158 (e.g., undercarboxylated/uncarboxylated osteocalcin) to treat or prevent disorders related to cognition in mammals. It is known that aging is frequently associated with mild to severe cognitive impairment. Aging is also associated with loss of bone mass. Since bone osteoblasts are a major source of osteocalcin, the findings disclosed herein support the use of osteocalcin to activate GPR158 and thus treat cognitive disorders associated with aging. In certain exemplary embodiments, the disorder is increased anxiety, increased depression, decreased memory, or decreased learning ability that occurs as a result of aging.

“Cognitive disorders” include conditions characterized by temporary or permanent loss, either total or partial, of the ability to learn, memorize, solve problems, process information, reason correctly, or recall information. In certain exemplary embodiments of the present disclosure, the cognitive disorder arises as a result of the normal aging process. In other exemplary embodiments, the cognitive disorder is the result of such factors as injury to the brain, specific neurodegenerative disease (e.g., Alzheimer's disease, Parkinson's diease,

Huntington's disease, amyotrophic lateral sclerosis), vascular conditions (e.g., stroke, ischemia), tumors or infections in the brain. When the cognitive disorder is memory loss, the loss may occur in short term or long term memory. Cognitive disorders also include various forms of dementia.

Preventing a disorder related to cognition in mammals means actively intervening as described herein prior to overt onset of the disorder to prevent or minimize the extent of the disorder or slow its course of development.

Treating a disorder related to cognition in mammals means actively intervening after onset of the disorder to slow down, ameliorate symptoms of, minimize the extent of, or reverse the disorder in a patient who is known or suspected of having the disorder.

A “patient” is a mammal, preferably a human, but can also be a companion animal such as dogs or cats, or farm animals such as horses, cattle, pigs, or sheep. In certain exemplary embodiments, the patient is a human who is more than 50, 55, 60, 65, 70, 75, or 80 years old. In certain exemplary embodiments, the patient is a human who is between 50 and 80 years old, between 55 and 75 years old, or between 60 and 70 years old. In certain exemplary embodiments, the patient is a human who is between 50 and 55 years old, between 55 and 60 years old, between 65 and 70 years old, between 70 and 75 years old, between 75 and 80 years old, between 80 and 85 years old, or between 85 and 90 years old.

A patient in need of treatment or prevention for a cognitive disorder includes a patient known or suspected of having or being at risk of developing a cognitive disorder.

Such a patient in need of treatment could be, e.g., a mammal known to have low undercarboxylated/uncarboxylated levels. Patients in need of treatment or prevention by the methods of the present disclosure include patients who are known to be in need of therapy to increase serum undercarboxylated/uncarboxylated levels in order to treat or prevent a cognitive disorder. In some exemplary embodiments, such patients might include mammals that have been identified as having a serum undercarboxylated/uncarboxylated level that is about 5%, about 15%, or about 50% lower than the serum undercarboxylated/uncarboxylated level in normal subjects.

A patient in need of treatment or prevention for a cognitive disorder by the methods of the present disclosure does not include a patient being administered the therapeutic agents described herein where the patient is being administered the therapeutic agents only for a purpose other than to treat or prevent a cognitive disorder. Thus, e.g., a patient in need of treatment or prevention for a cognitive disorder by the methods of the present disclosure does not include a patient being treated with osteocalcin only for the purpose of treating a bone mass disease, metabolic syndrome, glucose intolerance, type 1 diabetes, type 2 diabetes, atherosclerosis, or obesity. Nor does it include a patient being treated with osteocalcin only for the purpose of causing an increase in glucose tolerance, an increase in insulin production, an increase insulin sensitivity, an increase in pancreatic beta-cell proliferation, an increase in adiponectin serum level, a reduction of oxidized phospholipids, a regression of atherosclerotic plaques, a decrease in inflammatory protein biosynthesis, a reduction in plasma cholesterol, a reduction in vascular smooth muscle cell (VSMC) proliferation and number, or a decrease in the thickness of arterial plaque. A patient in need of treatment or prevention for a cognitive disorder by the methods of the present disclosure also does not include a patient being treated with osteocalcin that is not undercarboxylated/uncarboxylated osteocalcin.

In certain exemplary embodiments, the methods of the present disclosure comprise the step(s)/procedures(s) of identifying a patient in need of therapy for a cognitive disorder. Thus, the present disclosure provides a method comprising:

(a) identifying a patient in need of therapy for a cognitive disorder;

(b) administering to the patient a therapeutically effective amount of an agent that activates GPR158.

Other exemplary aspects of the present disclosure are directed to diagnostic methods based on detection of the level of undercarboxylated/uncarboxylated osteocalcin in a patient, which level is associated with disorders related to cognition in mammals. The diagnostic methods may be followed by the administration of a therapeutically effective amount of an agent that activates GPR158, e.g., undercarboxylated/uncarboxylated osteocalcin, to the patient.

In one exemplary aspect, the method of diagnosing a cognitive disorder in a patient can comprise (i) determining a patient level of undercarboxylated/uncarboxylated osteocalcin in a biological sample taken from the patient (ii) comparing the patient level of undercarboxylated/uncarboxylated osteocalcin and a control level of undercarboxylated/uncarboxylated osteocalcin, and (iii) if the patient level is significantly lower than the control level, then diagnosing the patient as having, or being at risk for, the cognitive disorder. A further step may then be to inform the patient or the patient's healthcare provider of the diagnosis. An even further step may be for the healthcare provider to administer a therapeutically effective amount of an agent that activates GPR158, e.g., undercarboxylated/uncarboxylated osteocalcin, to the patient.

Other exemplary aspects of the present disclosure are directed to diagnostic methods based on detection of decreased ratios of undercarboxylated/uncarboxylated vs carboxylated osteocalcin. Such ratios may be associated with disorders related to cognition in mammals. In one aspect, the method of diagnosing a disorder related to cognition in a patient comprises (i) determining a patient ratio of undercarboxylated/uncarboxylated vs. carboxylated osteocalcin in a biological sample taken from the patient (ii) comparing the patient ratio of undercarboxylated/uncarboxylated vs carboxylated osteocalcin and a control ratio of undercarboxylated/uncarboxylated vs carboxylated osteocalcin, and (iii) if the patient ratio is significantly lower than the control ratio, then the patient is diagnosed as having, or being at risk for, the disorder related to cognition. A further step may then be to inform the patient or the patient's healthcare provider of the diagnosis. An even further step may be for the healthcare provider to administer a therapeutically effective amount of an agent that activates GPR158, e.g., undercarboxylated/uncarboxylated osteocalcin, to the patient.

### Pharmaceutical Compositions for Use in Methods of Exemplary Embodiments

Exemplary embodiments of the present disclosure provide pharmaceutical compositions for use in the treatment of a cognitive disorder in mammals comprising an agent that activates GPR158. In certain exemplary embodiments, the agent inhibits the ability of GPR158 to signal through the inositol triphosphate pathway. The agent may be selected from the group consisting of small molecules, polypeptides, antibodies, and nucleic acids. The pharmaceutical compositions of the present disclosure provide an amount of the agent effective to treat or prevent a cognitive disorder in mammals. In certain exemplary embodiments, the pharmaceutical composition provides an amount of the agent effective to treat or prevent neurodegeneration associated with aging, anxiety, depression, memory loss, learning difficulties, and cognitive disorders associated with food deprivation during pregnancy.

In particular exemplary embodiments of the present disclosure, therapeutic agents that may be administered in the methods of the present disclosure include undercarboxylated osteocalcin or uncarboxylated osteocalcin, as well as antibodies, small molecules, antisense nucleic acids or siRNA that activate GPR158.

The therapeutic agents are generally administered in an amount sufficient to lessen cognitive loss due to neurodegeneration associated with aging, lessen anxiety, lessen depression, lessen memory loss, improve learning, or lessen cognitive disorders associated with food deprivation during pregnancy.

In certain exemplary embodiments, pharmaceutical compositions comprising undercarboxylated/uncarboxylated osteocalcin can be administered together with another therapeutic agent that is known to be useful for treating cognitive disorders in mammals. Examples of such other therapeutic agents include monoamine oxidase B inhibitors such as selegiline; vasodilators such as nicerogoline and vinpocetine; phosphatidylserine; propentofyline; anticholinesterases (cholinesterase inhibitors) such as tacrine, galantamine, rivastigmine, vinpocetine, donepezil (ARICEPT® (donepezil hydrochloride)), metrifonate, and physostigmine; lecithin; choline cholinomimetics such as milameline and xanomeline; ionotropic N-methyl-D-aspartate (NMDA) receptor antagonists such as memantine; anti-inflammatory drugs such as prednisolone, diclofenac, indomethacin, propentofyline, naproxen, rofecoxin, ibruprofen and suldinac; metal chelating agents such as cliquinol; Ginkgo biloba; bisphosophonates; selective oestrogen receptor modulators such as raloxifene and estrogen; beta and gamma secretase inhibitors; cholesterol-lowering drugs such as statins; calcitonin; risedronate; alendronate; and combinations thereof

In some exemplary embodiments, the agent that activates GPR158 such as undercarboxylated/uncarboxylated osteocalcin and the other therapeutic agent that is known to be useful for treating cognitive disorders in mammals are present in the same pharmaceutical composition. In other exemplary embodiments, the agent that activates GPR158 such as undercarboxylated/uncarboxylated osteocalcin and the other therapeutic agent that is known to be useful for treating cognitive disorders in mammals are administered in separate pharmaceutical compositions.

In other exemplary embodiments, agent that activates GPR158 such as undercarboxylated/uncarboxylated osteocalcin is the only active pharmaceutical ingredient present in the pharmaceutical compositions of the present disclosure.

Biologically active fragments or variants of the therapeutic agents are also within the scope of the present disclosure. By “biologically active” is meant capable of activating GPR158 such that GPR158 signals through the pathway that is activated when undercarboxylated/uncarboxylated osteocalcin binds to and activates GPR158.

“Biologically active” also refers to fragments or variants of osteocalcin that retain the ability of undercarboxylated/uncarboxylated osteocalcin to treat or prevent a cognitive disorder in mammals.

“Biologically active” also means capable of producing at least one effect in a mammal selected from the group consisting of lessening of cognitive loss due to neurodegeneration associated with aging, lessening of anxiety, lessening of depression, lessening of memory loss, improving learning, and lessening of cognitive disorders associated with food deprivation during pregnancy.

### Pharmaceutical Compositions Comprising Undercarboxylated/Uncarboxylated Osteocalcin

In a specific exemplary embodiment of the present disclosure, pharmaceutical compositions comprising undercarboxylated/uncarboxylated osteocalcin are provided for use in treating or preventing a cognitive disorder in a mammal.

“Undercarboxylated osteocalcin” means osteocalcin in which one or more of the Glu residues at positions Glu17, Glu21, and Glu24 of the amino acid sequence of the mature human osteocalcin having 49 amino acids, or at the positions corresponding to Glu17, Glu21 and Glu24 in other forms of osteocalcin, are not carboxylated. Undercarboxylated osteocalcin includes “uncarboxylated osteocalcin,” i.e., osteocalcin in which all three of the glutamic acid residues at positions 17, 21, and 24 are not carboxylated. Preparations of osteocalcin are considered to be “undercarboxylated osteocalcin” if more than about 10% of the total Glu residues at positions Glu17, Glu21, and Glu24 (taken together) in mature osteocalcin (or the corresponding Glu residues in other forms) of the preparation are not carboxylated. In particular preparations of undercarboxylated osteocalcin, more than about 20%, more than about 30%, more than about 40%, more than about 50%, more than about 60%, more than about 70%, more than about 80%, more than about 90%, more than about 95%, or more than about 99% of the total Glu residues at positions Glu17, Glu21, and Glu24 in mature osteocalcin (or the corresponding Glu residues in other forms) of the preparation are not carboxylated. In particularly preferred exemplary embodiments, essentially all of the

Glu residues at positions Glu17, Glu21 and Glu24 in mature osteocalcin (or the corresponding Glu residues in other forms) of the preparation are not carboxylated.

“Undercarboxylated/uncarboxylated osteocalcin” is used herein to refer collectively to undercarboxylated and uncarboxylated osteocalcin.

Human osteocalcin cDNA is the following sequence (SEQ ID NO:1)

cgcagccacc gagacaccat gagagccctc acactcctcg
ccctattggc cctggccgca ctttgcatcg ctggccaggc
aggtgcgaag cccagcggtg cagagtccag caaaggtgca
gcctttgtgt ccaagcagga gggcagcgag gtagtgaaga
gacccaggcg ctacctgtat caatggctgg gagccccagt
cccctacccg gatcccctgg agcccaggag ggaggtgtgt
gagctcaatc cggactgtga cgagttggct gaccacatcg
gctttcagga ggcctatcgg cgcttctacg gcccggtcta
gggtgtcgct ctgctggcct ggccggcaac cccagttctg
ctcctctcca ggcacccttc tttcctcttc cccttgccct
tgccctgacc tcccagccct atggatgtgg ggtccccatc
atcccagctg ctcccaaata aactccagaa gaggaatctg
aaaaaaaaaa aaaaaaaa

SEQ ID NO:1 encodes the pre-pro-sequence of human osteocalcin (SEQ ID NO:2)

MRALTLLALL ALAALCIAGQ AGAKPSGAES SKGAAFVSKQ
EGSEVVKRPR RYLYQWLGAP VPYPDPLEPR REVCELNPDC
DELADHIGFQ EAYRRFYGPV

Mature human osteocalcin protein is the last 49 amino acids of SEQ ID NO:2 (i.e., positions 52-100) with a predicted molecular mass of 5,800 kDa (Poser et al., 1980, J. Biol. Chem. 255:8685-8691). Mature human osteocalcin protein has the following sequence (SEQ ID NO:9):

YLYQWLGAPV PYPDPLEPRR EVCELNPDCD ELADHIGFQE
AYRRFYGPV

In this application, the amino acid positions of mature human osteocalcin are referred to. It will be understood that the amino acid positions of mature human osteocalcin correspond to those of SEQ ID NO:2 as follows: position 1 of mature human osteocalcin corresponds to position 52 of SEQ ID NO:2; position 2 of mature human osteocalcin corresponds to position 53 of SEQ ID NO:2, etc. In particular, positions 17, 21, and 24 of mature human osteocalcin correspond to positions 68, 72, and 75, respectively, of SEQ ID NO:2.

When positions in two amino acid sequences correspond, it is meant that the two positions align with each other when the two amino acid sequences are aligned with one another to provide maximum homology between them. This same concept of correspondence also applies to nucleic acids.

For example, in the two amino acid sequences AGLYSTVLMGRPS and GLVSTVLMGN, positions 2-11 of the first sequence correspond to positions 1-10 of the second sequence, respectively. Thus, position 2 of the first sequence corresponds to position 1 of the second sequence; position 4 of the first sequence corresponds to position 3 of the second sequence; etc. It should be noted that a position in one sequence may correspond to a position in another sequence, even if the positions in the two sequences are not occupied by the same amino acid.

“Osteocalcin” includes the mature protein and further includes biologically active fragments derived from full-length osteocalcin (SEQ ID NO:2) or the mature protein (SEQ ID NO:9), including various domains, as well as variants as described herein.

In one exemplary embodiment of the present disclosure, the pharmaceutical compositions for use in the methods of the present disclosure comprise a mammalian uncarboxylated osteocalcin. In a preferred embodiment of the present disclosure, the compositions for use in the methods of the present disclosure comprise human uncarboxylated osteocalcin having the amino acid sequence of SEQ ID NO:2, or portions thereof, and encoded for by the nucleic acid of SEQ ID NO:1, or portions thereof In some exemplary embodiments, the compositions for use in the methods of the present disclosure may comprise one or more of the human osteocalcin fragments described herein.

In an exemplary embodiment of the present disclosure, the compositions for use in the methods of the present disclosure comprise human uncarboxylated osteocalcin having the amino acid sequence of SEQ ID NO:9.

In a specific exemplary embodiment of the present disclosure, pharmaceutical compositions can be provided which can comprise human undercarboxylated osteocalcin which does not contain a carboxylated glutamic acid at one or more of positions corresponding to positions 17, 21, and 24 of mature human osteocalcin. A preferred form of osteocalcin for use in the methods of the present disclosure is mature human osteocalcin wherein at least one of the glutamic acid residues at positions 17, 21, and 24 is not carboxylated. In certain exemplary embodiments, the glutamic acid residue at position 17 is not carboxylated. Preferably, all three of the glutamic acid residues at positions 17, 21, and 24 are not carboxylated. The amino acid sequence of mature human osteocalcin is shown in SEQ ID NO:9.

The primary sequence of osteocalcin is highly conserved among species and it is one of the ten most abundant proteins in the human body, suggesting that its function is preserved throughout evolution. Conserved features include 3 Gla residues at positions 17, 21, and 24 and a disulfide bridge between Cys23 and Cys29. In addition, most species contain a hydroxyproline at position 9. The N-terminus of osteocalcin shows highest sequence variation in comparison to other parts of the molecule. The high degree of conservation of human and mouse osteocalcin underscores the relevance of the mouse as an animal model for the human, in both healthy and diseased states, and validates the therapeutic and diagnostic use of osteocalcin to treat or prevent disorders related to cognition in humans based on the experimental data derived from the mouse model disclosed herein.

The exemplary emnbodiment of the present disclosure also describe the use of polypeptide fragments of osteocalcin as agents to activate GPR158. Fragments can be derived from the full-length, naturally occurring amino acid sequence of osteocalcin (e.g., SEQ ID NO:2). Fragments may also be derived from mature osteocalcin (e.g., SEQ ID NO:9). The present disclosure also encompasses fragments of the variants of osteocalcin described herein. A fragment can comprise an amino acid sequence of any length that is biologically active.

Preferred fragments of osteocalcin include fragments containing Glu17, Glu21, and Glu24 of the mature protein. Also preferred are fragments of the mature protein missing the last 10 amino acids from the C-terminal end of the mature protein. Also preferred are fragments missing the first 10 amino acids from the N-terminal end of the mature protein. Also preferred is a fragment of the mature protein missing both the last 10 amino acids from the C-terminal end and the first 10 amino acids from the N-terminal end. Such a fragment comprises amino acids 62-90 of SEQ ID NO:2.

Other preferred fragments of osteocalcin for the pharmaceutical compositions of the present disclosure described herein include polypeptides comprising, consisting of, and/or consisting essentially of, the following sequences of amino acids:
positions 1-19 of mature human osteocalcin
positions 20-43 of mature human osteocalcin
positions 20-49 of mature human osteocalcin
positions 1-43 of mature human osteocalcin
positions 1-42 of mature human osteocalcin
positions 1-41 of mature human osteocalcin
positions 1-40 of mature human osteocalcin
positions 1-39 of mature human osteocalcin
positions 1-38 of mature human osteocalcin
positions 1-37 of mature human osteocalcin
positions 1-36 of mature human osteocalcin
positions 1-35 of mature human osteocalcin
positions 1-34 of mature human osteocalcin
positions 1-33 of mature human osteocalcin
positions 1-32 of mature human osteocalcin
positions 1-31 of mature human osteocalcin
positions 1-30 of mature human osteocalcin
positions 1-29 of mature human osteocalcin
positions 2-49 of mature human osteocalcin
positions 2-45 of mature human osteocalcin
positions 2-40 of mature human osteocalcin
positions 2-35 of mature human osteocalcin
positions 2-30 of mature human osteocalcin
positions 2-25 of mature human osteocalcin
positions 2-20 of mature human osteocalcin
positions 4-49 of mature human osteocalcin
positions 4-45 of mature human osteocalcin
positions 4-40 of mature human osteocalcin
positions 4-35 of mature human osteocalcin
positions 4-30 of mature human osteocalcin
positions 4-25 of mature human osteocalcin
positions 4-20 of mature human osteocalcin
positions 8-49 of mature human osteocalcin
positions 8-45 of mature human osteocalcin
positions 8-40 of mature human osteocalcin
positions 8-35 of mature human osteocalcin
positions 8-30 of mature human osteocalcin
positions 8-25 of mature human osteocalcin
positions 8-20 of mature human osteocalcin
positions 10-49 of mature human osteocalcin
positions 10-45 of mature human osteocalcin
positions 10-40 of mature human osteocalcin
positions 10-35 of mature human osteocalcin
positions 10-30 of mature human osteocalcin
positions 10-25 of mature human osteocalcin
positions 10-20 of mature human osteocalcin
positions 6-34 of mature human osteocalcin
positions 6-35 of mature human osteocalcin
positions 6-36 of mature human osteocalcin
positions 6-37 of mature human osteocalcin
positions 6-38 of mature human osteocalcin
positions 7-34 of mature human osteocalcin
positions 7-35 of mature human osteocalcin
positions 7-36 of mature human osteocalcin
positions 7-37 of mature human osteocalcin
positions 7-38 of mature human osteocalcin
positions 7-30 of mature human osteocalcin
positions 7-25 of mature human osteocalcin
positions 7-23 of mature human osteocalcin
positions 7-21 of mature human osteocalcin
positions 7-19 of mature human osteocalcin
positions 7-17 of mature human osteocalcin
positions 8-30 of mature human osteocalcin
positions 8-25 of mature human osteocalcin
positions 8-23 of mature human osteocalcin
positions 8-21 of mature human osteocalcin
positions 8-19 of mature human osteocalcin
positions 8-17 of mature human osteocalcin
positions 9-30 of mature human osteocalcin
positions 9-25 of mature human osteocalcin
positions 9-23 of mature human osteocalcin
positions 9-21 of mature human osteocalcin
positions 9-19 of mature human osteocalcin
positions 9-17 of mature human osteocalcin

It can be preferred that is a fragment comprising positions 1-36 of mature human osteocalcin. Another preferred fragment is a fragment comprising positions 20-49 of mature human osteocalcin. Other fragments can be designed to contain Pro13 to Tyr76 or Pro13 to Asn26 of mature human osteocalcin. Additionally, fragments containing the cysteine residues at positions 23 and 29 of mature human osteocalcin, and capable of forming a disulfide bond between those two cysteines, are useful.

Fragments can be discrete (not fused to other amino acids or polypeptides) or can be within a larger polypeptide. Further, several fragments can be comprised within a single larger polypeptide. In one embodiment, a fragment designed for expression in a host can have heterologous pre- and pro-polypeptide regions fused to the amino terminus of the osteocalcin fragment and/or an additional region fused to the carboxyl terminus of the fragment.

The exemplary use of the exemplary embodiments can be in the compositions and methods of the present disclosure that are variants of osteocalcin and the osteocalcin fragments described above. “Variants” refers to osteocalcin peptides that contain modifications in their amino acid sequences such as one or more amino acid substitutions, additions, deletions and/or insertions but that are still biologically active. In some instances, the antigenic and/or immunogenic properties of the variants are not substantially altered, relative to the corresponding peptide from which the variant was derived. Such modifications may be readily introduced using standard mutagenesis techniques, such as oligonucleotide directed site-specific mutagenesis as taught, for example, by Adelman et al., 1983, DNA 2:183, or by chemical synthesis. Variants and fragments are not mutually exclusive terms. Fragments also include peptides that may contain one or more amino acid substitutions, additions, deletions and/or insertions such that the fragments are still biologically active.

One particular type of variant that is within the scope of the present disclosure is a variant in which one of more of the positions corresponding to positions 17, 21, and 24 of mature human osteocalcin is occupied by an amino acid that is not glutamic acid. In some exemplary embodiments, the amino acid that is not glutamic acid is also not aspartic acid. Such variants are versions of undercarboxylated osteocalcin because at least one of the three positions corresponding to positions 17, 21, and 24 of mature human osteocalcin is not carboxylated glutamic acid, since at least one of those positions is not occupied by glutamic acid.

In particular exemplary embodiments of the present disclosure, osteocalcin variants canbe provided comprising the amino acid sequence

(SEQ ID NO: 10)
YL YQWLGAPV PYPDPLX₁PRR X₂VCX₃LNPDCD ELADHIGFQE
AYRRFYGPV

wherein

X₁, X₂ and X₃ are each independently selected from an amino acid or amino acid analog, with the proviso that if X₁, X₂ and X₃ are each glutamic acid, then X₁ is not carboxylated, or less than 50 percent of X₂ is carboxylated, and/or less than 50 percent of X₃ is carboxylated.

In certain exemplary embodiments, the osteocalcin variants comprise an amino acid sequence that is different from SEQ ID NO:10 at 1 to 7 positions other than X1, X2 and X3.

In other exemplary embodiments, the osteocalcin variants comprise an amino acid sequence that includes one or more amide backbone substitutions.

Fully functional variants typically contain only conservative variation or variation in non-critical residues or in non-critical regions. Functional variants can also contain substitutions of similar amino acids, which results in no change, or an insignificant change, in function. Alternatively, such substitutions may positively or negatively affect function to some degree. The activity of such functional osteocalcin variants can be determined using assays such as those described herein.

Variants can be naturally-occurring or can be made by recombinant means, or chemical synthesis, to provide useful and novel characteristics for undercarboxylated/uncarboxylated osteocalcin. For example, the variant osteocalcin polypeptides may have reduced immunogenicity, increased serum half-life, increased bioavailability, and/or increased potency. In particular exemplary embodiments, serum half-life is increased by substituting one or more of the native Arg residues at positions 19, 20, 43, and 44 of mature osteocalcin with another amino acid or an amino acid analog, e.g., β-dimethyl-arginine. Such substitutions can be combined with the other changes in the native amino acid sequence of osteocalcin described herein.

Provided for use in the pharmaceutical compositions and methods of the present disclosure are variants that are also derivatives of the osteocalcin and osteocalcin fragments described above. Derivatization is a technique used in chemistry which transforms a chemical compound into a product of similar chemical structure, called derivative. Generally, a specific functional group of the compound participates in the derivatization reaction and transforms the compound to a derivate of different reactivity, solubility, boiling point, melting point, aggregate state, functional activity, or chemical composition. Resulting new chemical properties can be used for quantification or separation of the derivatized compound or can be used to optimize the derivatized compound as a therapeutic agent. The well-known techniques for derivatization can be applied to the above-described osteocalcin and osteocalcin fragments. Thus, derivatives of the osteocalcin and osteocalcin fragments described above will contain amino acids that have been chemically modified in some way so that they differ from the natural amino acids.

Provided also can be osteocalcin mimetics. “Mimetic” refers to a synthetic chemical compound that has substantially the same structural and functional characteristics of a naturally or non-naturally occurring osteocalcin polypeptide, and includes, for instance, polypeptide- and polynucleotide-like polymers having modified backbones, side chains, and/or bases. Peptide mimetics are commonly used in the pharmaceutical industry as non-peptide drugs with properties analogous to those of the template peptide. Generally, mimetics are structurally similar (i.e., have the same shape) to a paradigm polypeptide that has a biological or pharmacological activity, but one or more polypeptide linkages are replaced. The mimetic can be either entirely composed of synthetic, non-natural analogues of amino acids or is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity.

By way of examples that can be adapted to osteocalcin by those skilled in the art: Cho et al., 1993, Science 261:1303-1305 discloses an “unnatural biopolymer” consisting of chiral aminocarbonate monomers substituted with a variety of side chains, synthesis of a library of such polymers, and screening for binding affinity to a monoclonal antibody. Simon et al., 1992, Proc. Natl. Acad. Sci. 89:9367-9371 discloses a polymer consisting of N-substituted glycines (“peptoids”) with diverse side chains. Schumacher et al, 1996, Science 271:1854-1857 discloses D-peptide ligands identified by screening phage libraries of L-peptides against proteins synthesized with D-amino acids and then synthesizing a selected L-peptide using D-amino acids. Brody et al., 1999, Mol. Diagn. 4:381-8 describes generation and screening of hundreds to thousands of aptamers.

A particular type of osteocalcin variant within the scope of the present disclosure is an osteocalcin mimetic in which one or more backbone amides is replaced by a different chemical structure or in which one or more amino acids are replaced by an amino acid analog. In aparticular exemplary embodiment, the osteocalcin mimetic is a retroenantiomer of uncarboxylated human osteocalcin.

Osteocalcin, as well as its fragments and variants, is optionally produced by chemical synthesis or recombinant methods and may be produced as a modified osteocalcin molecule (i.e., osteocalcin fragments or variants) as described herein. Osteocalcin polypeptides can be produced by any conventional means (Houghten, 1985, Proc. Natl. Acad. Sci. USA 82:5131-5135). Simultaneous multiple peptide synthesis is described in U.S. Pat. No. 4,631,211 and can also be used. When produced recombinantly, osteocalcin may be produced as a fusion protein, e.g., a GST-osteocalcin fusion protein.

Undercarboxylated/uncarboxylated osteocalcin molecules that can be used in the methods of the present disclosure include proteins substantially homologous to human osteocalcin, including proteins derived from another organism, i.e., an ortholog of human osteocalcin. One particular ortholog is mouse osteocalcin. Mouse osteocalcin gene 1 cDNA is SEQ ID NO:3, having the following sequence:

agaacagaca agtcccacac agcagcttgg cccagaccta
gcagacacca tgaggaccat ctttctgctc actctgctga
ccctggctgc gctctgtctc tctgacctca cagatgccaa
gcccagcggc cctgagtctg acaaagcctt catgtccaag
caggagggca ataaggtagt gaacagactc cggcgctacc
ttggagcctc agtccccagc ccagatcccc tggagcccac
ccgggagcag tgtgagctta accctgcttg tgacgagcta
tcagaccagt atggcttgaa gaccgcctac aaacgcatct
atggtatcac tatttaggac ctgtgctgcc ctaaagccaa
actctggcag ctcggctttg gctgctctcc gggacttgat
cctccctgtc ctctctctct gccctgcaag tatggatgtc
acagcagctc caaaataaag ttcagatgag gaagtgcaaa
aaaaaaaaaa aaaa

Mouse osteocalcin gene 2 cDNA is SEQ ID NO:4, having the following sequence:

gaacagacaa gtcccacaca gcagcttggt gcacacctag
cagacaccat gaggaccctc tctctgctca ctctgctggc
cctggctgcg ctctgtctct ctgacctcac agatcccaag
cccagcggcc ctgagtctga caaagccttc atgtccaagc
aggagggcaa taaggtagtg aacagactcc ggcgctacct
tggagcctca gtccccagcc cagatcccct ggagcccacc
cgggagcagt gtgagcttaa ccctgcttgt gacgagctat
cagaccagta tggcttgaag accgcctaca aacgcatcta
cggtatcact atttaggacc tgtgctgccc taaagccaaa
ctctggcagc tcggctttgg ctgctctccg ggacttgatc
ctccctgtcc tctctctctg ccctgcaagt atggatgtca
cagcagctcc aaaataaagt tcagatgagg

The amino acid sequence encoded by mouse osteocalcin gene 1 and gene 2 is SEQ ID NO:5, with the following sequence:

MRTLSLLTLL ALAALCLSDL TDPKPSGPES DKAFMSKQEG
NKVVNRLRRY LGASVPSPDP LEPTREQCEL NPACDELSDQ
YGLKTAYKRI YGITI

As used herein, two proteins can be, e.g., substantially homologous when their amino acid sequences are at least about 70-75% homologous. Typically the degree of homology is at least about 80-85%, and most typically at least about 90-95%, 97%, 98% or 99% or more. “Homology” between two amino acid sequences or nucleic acid sequences can be determined by using the algorithms disclosed herein. These exemplary procedures/algorithms can also be used to determine percent identity between two amino acid sequences or nucleic acid sequences.

In a specific embodiment of the present disclosure, the undercarboxylated/uncarboxylated osteocalcin is an osteocalcin molecule sharing at least 80% homology with the human osteocalcin of SEQ ID:2 or a portion of SEQ ID:2 that is at least 8 amino acids long. In another embodiment, the undercarboxylated/uncarboxylated osteocalcin is an osteocalcin molecule sharing at least 80%, at least 90%, at least 95%, or at least 97% amino acid sequence identity with the human osteocalcin of SEQ ID:2 or a portion of SEQ ID:2 that is at least 8 amino acids long. Homologous sequences include those sequences that are substantially identical. In preferred exemplary embodiments, the homology or identity is over the entire length of mature human osteocalcin.

To determine the percent homology or percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). Preferably, the length of a reference sequence aligned for comparison purposes is at least 30%, preferably at least 40%, more preferably at least 50%, even more preferably at least 60%, and even more preferably at least 70%, 80%, or 90% or more of the length of the sequence that the reference sequence is compared to. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The present disclosure also encompasses polypeptides having a lower degree of identity but which have sufficient similarity so as to perform one or more of the same functions performed by undercarboxylated/uncarboxylated osteocalcin, e.g., binding to and activating GPR158. Similarity is determined by considering conserved amino acid substitutions. Such substitutions are those that substitute a given amino acid in a polypeptide by another amino acid of like characteristics. Conservative substitutions are likely to be phenotypically silent. Guidance concerning which amino acid changes are likely to be phenotypically silent may be found in Bowie et al., 1990, Science 247:1306-1310.

Examples of conservative substitutions are the replacements, one for another, among the hydrophobic amino acids Ala, Val, Leu, and Ile; interchange of the hydroxyl residues Ser and Thr; exchange of the acidic residues Asp and Glu; substitution between the amide residues Asn and Gln; exchange of the basic residues Lys, His and Arg; replacements among the aromatic residues Phe, Trp and Tyr; exchange of the polar residues Gln and Asn; and exchange of the small residues Ala, Ser, Thr, Met, and Gly.

The comparison of sequences and determination of percent identity and homology between two osteocalcin polypeptides can be accomplished using a mathematical algorithm. See, for example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, HG., eds., Humana Press, New Jersey, 1994; Sequence Analysis in

Molecular Biology, van Heinje, G., Academic Press, 1987; and Sequence Analysis Primer, Gribskov, M. and Devereux, J., eds., M Stockton Press, New York, 1991. A non-limiting example of such a mathematical algorithm is described in Karlin et al., 1993, Proc. Natl. Acad. Sci. USA 90:5873-5877.

The percent identity or homology between two osteocalcin amino acid sequences may be determined using the Needleman et al., 1970, J. Mol. Biol. 48:444-453 algorithm.

A substantially homologous osteocalcin, according to the present disclosure, may also be a polypeptide encoded by a nucleic acid sequence capable of hybridizing to the human osteocalcin nucleic acid sequence under highly stringent conditions, e.g., hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel et al., eds., 1989, Current Protocols in Molecular Biology, Vol. I, Green Publishing Associates, Inc., and John Wiley & Sons, Inc., New York, at p. 2.10.3) and encoding a functionally equivalent gene product; or under less stringent conditions, such as moderately stringent conditions, e.g., washing in 0.2×SSC/0.1% SDS at 42° C. (Ausubel et al., 1989 supra), yet which still encodes a biologically active undercarboxylated/uncarboxylated osteocalcin.

A substantially homologous osteocalcin according to the present disclosure may also be a polypeptide encoded by a nucleic acid sequence capable of hybridizing to a sequence having at least 70-75%, typically at least about 80-85%, and most typically at least about 90-95%, 97%, 98% or 99% identity to the human osteocalcin nucleic acid sequence, under stringent conditions, e.g., hybridization to filter-bound DNA in 0.5 M NaHPO4, 7% sodium dodecyl sulfate (SDS), 1 mM EDTA at 65° C., and washing in 0.1×SSC/0.1% SDS at 68° C. (Ausubel F.M. et al., eds., 1989, Current Protocols in Molecular Biology, Vol. I, Green Publishing Associates, Inc., and John Wiley & sons, Inc., New York, at p. 2.10.3) and encoding a functionally equivalent gene product; or under less stringent conditions, such as moderately stringent conditions, e.g., washing in 0.2×SSC/0.1% SDS at 42° C. (Ausubel et al., 1989 supra), yet which still encodes a biologically active undercarboxylated/uncarboxylated osteocalcin.

It will be understood that a biologically active fragment or variant of human osteocalcin may contain a different number of amino acids than native human osteocalcin.

Accordingly, the position number of the amino acid residues corresponding to positions 17, 21, and 24 of mature human osteocalcin may differ in the fragment or variant. One skilled in the art would easily recognize such corresponding positions from a comparison of the amino acid sequence of the fragment or variant with the amino acid sequence of mature human osteocalcin.

Peptides corresponding to fusion proteins in which full length osteocalcin, mature osteocalcin, or an osteocalcin fragment or variant is fused to an unrelated protein or polypeptide are also within the scope of the present disclosure and can be designed on the basis of the osteocalcin nucleotide and amino acid sequences disclosed herein. Such fusion proteins include fusions to an enzyme, fluorescent protein, or luminescent protein which provides a marker function. In a preferred embodiment of the present disclosure, the fusion protein comprises fusion to a polypeptide capable of targeting the osteocalcin to a particular target cell or location in the body. For example, osteocalcin polypeptide sequences may be fused to a ligand molecule capable of targeting the fusion protein to a cell expressing the receptor for said ligand. In a particular embodiment, osteocalcin polypeptide sequences may be fused to a ligand capable of targeting the fusion protein to specific neurons in the brain of a mammal.

Osteocalcin can also be made as part of a chimeric protein for drug screening or use in making recombinant protein. These chimeric proteins comprise an osteocalcin peptide sequence linked to a heterologous peptide having an amino acid sequence not substantially homologous to the osteocalcin. The heterologous peptide can be fused to the N-terminus or C-terminus of osteocalcin or can be internally located. In one embodiment, the fusion protein does not affect osteocalcin function. For example, the fusion protein can be a GST-fusion protein in which the osteocalcin sequences are fused to the N- or C-terminus of the GST sequences. Other types of fusion proteins include, but are not limited to, enzymatic fusion proteins, for example beta-galactosidase fusions, yeast two-hybrid GAL-4 fusions, poly-His fusions and Ig fusions. Such fusion proteins, particularly poly-His fusions, can facilitate the purification of recombinant osteocalcin. In certain host cells (e.g., mammalian host cells), expression and/or secretion of a protein can be increased by using a heterologous signal sequence. Therefore, the fusion protein may contain a heterologous signal sequence at its N-terminus.

Those skilled in art would understand how to adapt well-known techniques for use with osteocalcin. For example, European Patent Publication No. 0 464 533 discloses fusion proteins comprising various portions of immunoglobulin constant regions (Fc regions). The Fc region is useful in therapy and diagnosis and thus results, for example, in improved pharmacokinetic properties (see, e.g., European Patent Publication No. 0 232 262). In drug discovery, for example, human proteins have been fused with Fc regions for the purpose of high-throughput screening assays to identify antagonists (Bennett et al., 1995, J. Mol. Recog. 8:52-58 and Johanson et al., 1995, J. Biol. Chem. 270:9459-9471). Thus, various exemplary embodiments of this disclosure also utilize soluble fusion proteins containing an osteocalcin polypeptide and various portions of the constant regions of heavy or light chains of immunoglobulins of various subclasses (e.g., IgG, IgM, lgA, IgE,1gB). Preferred as immunoglobulin is the constant part of the heavy chain of human IgG, particularly IgG1, where fusion takes place at the hinge region. For some uses, it is desirable to remove the Fc region after the fusion protein has been used for its intended purpose. In a particular embodiment, the Fc part can be removed in a simple way by a cleavage sequence, which is also incorporated and can be cleaved, e.g., with factor Xa.

A chimeric or fusion protein can be produced by standard recombinant DNA techniques. For example, DNA fragments coding for the different protein sequences can be ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., 1992, Current Protocols in Molecular Biology). Moreover, many expression vectors are commercially available that already encode a fusion moiety (e.g., a GST protein). An osteocalcin-encoding nucleic acid can be cloned into such an expression vector such that the fusion moiety is linked in-frame to osteocalcin.

Chimeric osteocalcin proteins can be produced in which one or more functional sites are derived from a different isoform, or from another osteocalcin molecule from another species. Sites also could be derived from osteocalcin-related proteins that occur in the mammalian genome but which have not yet been discovered or characterized.

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art.

Accordingly, the osteocalcin polypeptides useful in the methods of the present disclosure also encompass derivatives which contain a substituted non-naturally occurring amino acid residue that is not one encoded by the genetic code, in which a substituent group is included, in which the mature polypeptide is fused with another compound, such as a compound to increase the half-life of the polypeptide (for example, polyethylene glycol), or in which the additional amino acids are fused to the osteocalcin polypeptide, such as a leader or secretory sequence or a sequence for purification of the osteocalcin polypeptide or a pro-protein sequence.

Undercarboxylated/uncarboxylated osteocalcin can be modified according to known methods in medicinal chemistry to increase its stability, half-life, uptake or efficacy. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

In a specific exemplary embodiment of the present disclosure, modifications may be made to the osteocalcin to reduce susceptibility to proteolysis at residue Arg43 as a means for increasing serum half life. Such modifications include, for example, the use of retroenantioisomers, D-amino acids, or other amino acid analogs.

Acylation of the N-terminal amino group can be accomplished using a hydrophilic compound, such as hydroorotic acid or the like, or by reaction with a suitable isocyanate, such as methylisocyanate or isopropylisocyanate, to create a urea moiety at the N-terminus. Other agents can also be N-terminally linked that will increase the duration of action of the osteocalcin derivative.

Reductive amination is the process by which ammonia is condensed with aldehydes or ketones to form imines which are subsequently reduced to amines. Reductive amination is a useful method for conjugating undercarboxylated/uncarboxylated osteocalcin and its fragments or variants to polyethylene glycol (PEG). Covalent linkage of PEG to undercarboxylated/uncarboxylated osteocalcin and its fragments and variants may result in conjugates with increased water solubility, altered bioavailability, pharmacokinetics, immunogenic properties, and biological activities. See, e.g., Bentley et al., 1998, J. Pharm. Sci. 87:1446-1449.

Several particularly common modifications that may be applied to undercarboxylated/uncarboxylated osteocalcin and its fragments and variants such as glycosylation, lipid attachment, sulfation, hydroxylation and ADP-ribosylation are described in most basic texts, such as Proteins-Structure and Molecular Properties, 2nd ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al., 1990, Meth. Enzymol. 182:626-646 and Rattan et al., 1992, Ann. New York Acad. Sci. 663:48-62.

As is also well known, polypeptides are not always entirely linear. For instance, polypeptides may be branched as a result of ubiquitination, and they may be circular, with or without branching, generally as a result of post-translation events, including natural processing events and events brought about by human manipulation which do not occur naturally. Circular, branched and branched circular polypeptides may be synthesized by non-translational natural processes and by synthetic methods. Well-known techniques for preparing such non-linear polypeptides may be adapted by those skilled in the art to produce non-linear osteocalcin polypeptides.

Modifications can occur anywhere in the undercarboxylated/uncarboxylated osteocalcin and its fragments and variants, including the peptide backbone, the amino acid side-chains and the amino or carboxyl termini. Blockage of the amino or carboxyl group in a polypeptide, or both, by a covalent modification, is common in naturally-occurring and synthetic polypeptides and may be applied to the undercarboxylated/uncarboxylated osteocalcin or its fragments and variants used in the present disclosure. For instance, the amino terminal residue of polypeptides made in E. coli, prior to proteolytic processing, almost invariably will be N-formylmethionine. Thus, the use of undercarboxylated/uncarboxylated osteocalcin and its fragments and variants with N-formylmethionine as the amino terminal residue are within the scope of the present disclosure.

A brief description of various protein modifications that come within the scope of this disclosure are set forth in the table below:

TABLE 1

| Protein Modification | Description |
|---|---|
| Acetylation | Acetylation of N-terminus or ε-lysines. Introducing an acetyl |
| | group into a protein, specifically, the substitution of an acetyl |
| | group for an active hydrogen atom. |
| | A reaction involving the replacement of the hydrogen atom |
| | of a hydroxyl group with an acetyl group (CH₃CO) yields a |
| | specific ester, the acetate. Acetic anhydride is commonly |
| | used as an acetylating agent, which reacts with free hydroxyl |
| | groups. |
| | Acylation may facilitate addition of other functional groups. |
| | A common reaction is acylation of e.g., conserved lysine |
| | residues with a biotin appendage. |
| ADP-ribosylation | Covalently linking proteins or other compounds via an |
| | arginine-specific reaction. |
| Alkylation | Alkylation is the transfer of an alkyl group from one |
| | molecule to another. The alkyl group may be transferred as |
| | an alkyl carbocation, a free radical or a carbanion (or their |
| | equivalents). Alkylation is accomplished by using certain |
| | functional groups such as alkyl electrophiles, alkyl |
| | nucleophiles or sometimes alkyl radicals or carbene acceptors. |
| | A common example is methylation (usually at a lysine or |
| | arginine residue). |
| Amidation | Reductive animation of the N-terminus Methods for |
| | amidation of insulin are described in U.S. Pat. No. 4,489,159. |
| Carbamylation | Nigen et al. describes a method of carbamylating hemoglobin. |
| Citrullination | Citrullination involves the addition of citrulline amino acids |
| | to the arginine residues of a protein, which is catalyzed by |
| | peptidylarginine deaminase enzymes (PADs). This generally |
| | converts a positively charged arginine into a neutral citrulline |
| | residue, which may affect the hydrophobicity of the protein |
| | (and can lead to unfolding). |
| Condensation of amines | Such reactions, may be used, e.g., to attach a peptide to other |
| with aspartate or glutamate | proteins labels. |
| Covalent attachment | Flavin mononucleotide (FAD) may be covalently attached to |
| of flavin | serine and/or threonine residues. May be used, e.g., as a |
| | light-activated tag. |
| Covalent attachment of | A heme moiety is generally a prosthetic group that consists |
| heme moiety | of an iron atom contained in the center of a large heterocyclic |
| | organic ring, which is referred to as a porphyrin. The heme |
| | moiety may be used, e.g., as a tag for the peptide. |
| Attachment of a nucleotide | May be used as a tag or as a basis for further derivatising a |
| or nucleotide derivative | peptide. |
| Cross-linking | Cross-linking is a method of covalently joining two proteins. |
| | Cross-linkers contain reactive ends to specific functional |
| | groups (primary amines, sulfhydryls, etc.) on proteins or |
| | other molecules. Several chemical groups may be targets for |
| | reactions in proteins and peptides For example, Ethylene |
| | glycol bis[succinimidylsuccinate, Bis[2- |
| | (succinimidooxycarbonyloxy)ethyl]sulfone, and |
| | Bis[sulfosuccinimidyl] suberate link amines to amines. |
| Cyclization | For example, cyclization of amino acids to create optimized |
| | delivery forms that are resistant to, e.g., aminopeptidases |
| | (e.g., formation of pyroglutamate, a cyclized form of |
| | glutamic acid). |
| Disulfide bond formation | Disulfide bonds in proteins are formed by thiol-disulfide |
| | exchange reactions, particularly between cysteine residues |
| | (e.g., formation of cystine). |
| Demethylation | See, e.g., U.S. Pat. No. 4,250,088 (Process for demethylating lignin). |
| Formylation | The addition of a formyl group to, e.g., the N-terminus of a |
| | protein. See, e.g., U.S. Pat. Nos. 4,059,589, 4,801,742, |
| | and 6,350,902. |
| Glycylation | The covalent linkage of one to more than 40 glycine residues |
| | to the tubulin C-terminal tail. |
| Glycosylation | Glycosylation may be used to add saccharides (or |
| | polysaccharides) to the hydroxy oxygen atoms of serine and |
| | threonine side chains (which is also known as O-linked |
| | Glycosylation). Glycosylation may also be used to add |
| | saccharides (or polysaccharides) to the amide nitrogen of |
| | asparagine side chains (which is also known as N-linked |
| | Glycosylation), e.g., via oligosaccharyl transferase. |
| GPI anchor formation | The addition of glycosylphosphatidylinositol to the C- |
| | terminus of a protein. GPI anchor formation involves the |
| | addition of a hydrophobic phosphatidylinositol group- |
| | linked through a carbohydrate containing linker (e.g., |
| | glucosamine and mannose linked to phosphoryl |
| | ethanolamine residue)-to the C-terminal amino acid of a |
| | protein. |
| Hydroxylation | Chemical process that introduces one or more hydroxyl |
| | groups (—OH) into a protein (or radical). Hydroxylation |
| | reactions are typically catalyzed by hydroxylases. Proline is |
| | the principal residue to be hydroxylated in proteins, which |
| | occurs at the C^{γ} atom, forming hydroxyproline (Hyp). In |
| | some cases, proline may be hydroxylated at its C^{β} atom. |
| | Lysine may also be hydroxylated on its C^{δ} atom, forming |
| | hydroxylysine (Hyl). These three reactions are catalyzed by |
| | large, multi-subunit enzymes known as prolyl 4-hydroxylase, |
| | prolyl 3-hydroxylase and lysyl 5-hydroxylase, respectively. |
| | These reactions require iron (as well as molecular oxygen |
| | and α-ketoglutarate) to carry out the oxidation, and use |
| | ascorbic acid to return the iron to its reduced state. |
| Iodination | See, e.g., U.S. Pat. No. 6,303,326 for a disclosure of an enzyme |
| | that is capable of iodinating proteins. U.S. Pat. No. 4,448,764 |
| | discloses, e.g., a reagent that may be used to iodinate proteins. |
| ISGylation | Covalently linking a peptide to the ISG15 (Interferon- |
| | Stimulated Gene 15) protein, for, e.g., modulating immune |
| | response. |
| Methylation | Reductive methylation of protein amino acids with |
| | formaldehyde and sodium cyanoborohydride has been shown |
| | to provide up to 25% yield of N-cyanomethyl (—CH₂CN) |
| | product. The addition of metal ions, such as Ni²⁺, which |
| | complex with free cyanide ions, improves reductive |
| | methylation yields by suppressing by-product formation. |
| | The N-cyanomethyl group itself, produced in good yield |
| | when cyanide ion replaces cyanoborohydride, may have |
| | some value as a reversible modifier of amino groups in |
| | proteins. (Gidley et al.) Methylation may occur at the |
| | arginine and lysine residues of a protein, as well as the N- |
| | and C-terminus thereof. |
| Myristoylation | Myristoylation involves the covalent attachment of a |
| | myristoyl group (a derivative of myristic acid), via an amide |
| | bond, to the alpha-amino group of an N-terminal glycine |
| | residue. This addition is catalyzed by the N- |
| | myristoyltransferase enzyme. |
| Oxidation | Oxidation of cysteines. |
| | Oxidation of N-terminal Serine or Threonine residues |
| | (followed by hydrazine or aminooxy condensations). |
| | Oxidation of glycosylations (followed by hydrazine or |
| | aminooxy condensations). |
| Palmitoylation | Palmitoylation is the attachment of fatty acids, such as |
| | palmitic acid, to cysteine residues of proteins. |
| | Palmitoylation increases the hydrophobicity of a protein. |
| (Poly)glutamylation | Polyglutamylation occurs at the glutamate residues of a |
| | protein. Specifically, the gamma-carboxy group of a |
| | glutamate will form a peptide-like bond with the amino |
| | group of a free glutamate whose alpha-carboxy group may be |
| | extended into a polyglutamate chain. The glutamylation |
| | reaction is catalyzed by a glutamylase enzyme (or removed |
| | by a deglutamylase enzyme). Polyglutamylation has been |
| | carried out at the C-terminus of proteins to add up to about |
| | six glutamate residues. Using such a reaction, Tubulin and |
| | other proteins can be covalently linked to glutamic acid residues. |
| Phosphopantetheinylation | The addition of a 4′-phosphopantetheinyl group. |
| Phosphorylation | A process for phosphorylation of a protein or peptide by |
| | contacting a protein or peptide with phosphoric acid in the |
| | presence of a non-aqueous apolar organic solvent and |
| | contacting the resultant solution with a dehydrating agent is |
| | disclosed e.g., in U.S. Pat. No. 4,534,894. Insulin products are |
| | described to be amenable to this process. See, e.g., U.S. Pat. No. |
| | 4,534,894. Typically, phosphorylation occurs at the serine, |
| | threonine, and tyrosine residues of a protein. |
| Prenylation | Prenylation (or isoprenylation or lipidation) is the addition of |
| | hydrophobic molecules to a protein. Protein prenylation |
| | involves the transfer of either a farnesyl (linear grouping of |
| | three isoprene units) or a geranyl-geranyl moiety to C- |
| | terminal cysteine (s) of the target protein. |
| Proteolytic Processing | Processing, e.g., cleavage of a protein at a peptide bond. |
| Selenoylation | The exchange of, e.g., a sulfur atom in the peptide for |
| | selenium, using a selenium donor, such as selenophosphate. |
| Sulfation | Processes for sulfating hydroxyl moieties, particularly |
| | tertiary amines, are described in, e.g., U.S. Pat. No. 6,452,035. A |
| | process for sulphation of a protein or peptide by contacting |
| | the protein or peptide with sulphuric acid in the presence of a |
| | non-aqueous apolar organic solvent and contacting the |
| | resultant solution with a dehydrating agent is disclosed. |
| | Insulin products are described to be amenable to this process. |
| | See, e.g., U.S. Pat. No. 4,534,894. |
| SUMOylation | Covalently linking a peptide a SUMO (small ubiquitin- |
| | related Modifier) protein, for, e.g., stabilizing the peptide. |
| Transglutamination | Covalently linking other protein (s) or chemical groups (e.g., |
| | PEG) via a bridge at glutamine residues |
| tRNA-mediated | For example, the site-specific modification (insertion) of an |
| addition of amino acids | amino acid analog into a peptide. |
| (e.g., arginylation) | |
| Ubiquitination | The small peptide ubiquitin is covalently linked to, e.g., |
| | lysine residues of a protein. The ubiquitin-proteasome |
| | system can be used to carryout such reaction. See, e.g., U.S. |
| | 2007-0059731. |

Theexemplary emebodiments of the present disclosure also encompasses the use of prodrugs of agents that activate GPR158 such as undercarboxylated/uncarboxylated osteocalcin or derivative or variant thereof that can be produced by esterifying the carboxylic acid functions of the agents that activate GPR158 such as undercarboxylated/uncarboxylated osteocalcin or derivative or variant thereof with a lower alcohol, e.g., methanol, ethanol, propanol, isopropanol, butanol, etc. The use of prodrugs of the agents that activate GPR158 such as undercarboxylated/uncarboxylated osteocalcin or derivative or variant thereof that are not esters is also contemplated. For example, pharmaceutically acceptable carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters of the agents that activate GPR158 such as undercarboxylated/uncarboxylated osteocalcin or derivative or variant thereof are also contemplated. In some exemplary embodiments, the prodrugs will contain a biohydrolyzable moiety (e.g., a biohydrolyzable amide, biohydrolyzable carbamate, biohydrolyzable carbonate, biohydrolyzable ester, biohydrolyzable phosphate, or biohydrolyzable ureide analog). Guidance for the preparation of prodrugs of the undercarboxylated/uncarboxylated osteocalcin or derivative or variant thereof disclosed herein can be found in publications such as Design of Prodrugs, Bundgaard, A. Ed., Elsevier, 1985; Design and Application of Prodrugs, A Textbook of Drug Design and Development, Krosgaard-Larsen and H. Bundgaard, Ed., 1991, Chapter 5, pages 113-191; and Bundgaard, H., Advanced Drug Delivery Review, 1992, 8, pages 1-38.

To practice the methods of the present disclosure, it may be desirable to recombinantly express osteocalcin, e.g., by recombinantly expressing a cDNA sequence encoding osteocalcin. The cDNA sequence and deduced amino acid sequence of human osteocalcin is represented in SEQ ID NO:1 and SEQ ID NO:2. Osteocalcin nucleotide sequences may be isolated using a variety of different methods known to those skilled in the art. For example, a cDNA library constructed using RNA from a tissue known to express osteocalcin can be screened using a labeled osteocalcin probe. Alternatively, a genomic library may be screened to derive nucleic acid molecules encoding osteocalcin. Further, osteocalcin nucleic acid sequences may be derived by performing a polymerase chain reaction (PCR) using two oligonucleotide primers designed on the basis of known osteocalcin nucleotide sequences. The template for the reaction may be cDNA obtained by reverse transcription of mRNA prepared from cell lines or tissue known to express osteocalcin.

While the osteocalcin polypeptides and peptides can be chemically synthesized (e.g., see Creighton, 1983, Proteins: Structures and Molecular Principles, W.H. Freeman & Co., N.Y.), large polypeptides derived from osteocalcin and the full length osteocalcin itself may be advantageously produced by recombinant DNA technology using techniques well known in the art for expressing a nucleic acid. Such methods can be used to construct expression vectors containing the osteocalcin nucleotide sequences and appropriate transcriptional and translational control signals. These methods include, for example, in vitro recombinant DNA techniques, synthetic techniques, and in vivo genetic recombination. See, for example, the techniques described in Ausubel et al., 1989, supra.

A variety of host-expression vector systems may be utilized to express the osteocalcin nucleotide sequences. In a preferred embodiment, the osteocalcin peptide or polypeptide is secreted and may be recovered from the culture media.

Appropriate expression systems can be chosen to ensure that the correct modification, processing and subcellular localization of the osteocalcin protein occurs. To this end, bacterial host cells are useful for expression of osteocalcin, as such cells are unable to carboxylate osteocalcin.

The isolated osteocalcin can be purified from cells that naturally express it, e.g., osteoblasts, or purified from cells that naturally express osteocalcin but have been recombinantly modified to overproduce osteocalcin, or purified from cells that that do not naturally express osteocalcin but have been recombinantly modified to express osteocalcin. In a particular embodiment, a recombinant cell has been manipulated to activate expression of the endogenous osteocalcin gene. For example, International Patent Publications WO 99/15650 and WO 00/49162 describe a method of expressing endogenous genes termed random activation of gene expression (RAGE), which can be used to activate or increase expression of endogenous osteocalcin. The RAGE methodology involves non-homologous recombination of a regulatory sequence to activate expression of a downstream endogenous gene. Alternatively, International Patent Publications WO 94/12650, WO 95/31560, and WO 96/29411, as well as U.S. Pat. No. 5,733,761 and U.S. Pat. No. 6,270,985, describe a method of increasing expression of an endogenous gene that involves homologous recombination of a DNA construct that includes a targeting sequence, a regulatory sequence, an exon, and a splice-donor site. Upon homologous recombination, a downstream endogenous gene is expressed. The methods of expressing endogenous genes described in the foregoing patents are hereby expressly incorporated by reference herein.

In certain exemplary embodiments of methods of the present disclosure, the therapeutic agent that activates GPR158 is administered to a patient in a dosage range of from about 0.5 μg/kg/day to about 100 mg/kg/day, from about 1 μg/kg/day to about 90 mg/kg/day, from about 5 μg/kg/day to about 85 mg/kg/day, from about 10 μg/kg/day to about 80 mg/kg/day, from about 20 μg/kg/day to about 75 mg/kg/day, from about 50 μg/kg/day to about 70 mg/kg/day, from about 150 μg/kg/day to about 65 mg/kg/day, from about 250 μg/kg/day to about 50 mg/kg/day, from about 500 μg/kg/day to about 50 mg/kg/day, from about 1 mg/kg/day to about 50 mg/kg/day, from about 5 mg/kg/day to about 40 mg/kg/day, from about 10 mg/kg/day to about 35 mg/kg/day, from about 15 mg/kg/day to about 30 mg/kg/day, from about 5 mg/kg/day to about 16 mg/kg/day, or from about 5 mg/kg/day to about 15 mg/kg/day.

In certain exemplary embodiments of methods of the present disclosure, the therapeutic agent that activates GPR158 is administered to a patient in a dosage range of from about 0.5 μg/kg/day to about 100 μg/kg/day, from about 1 μg/kg/day to about 80 μg/kg/day, from about 3 μg/kg/day to about 50 μg/kg/day, or from about 3 μg/kg/day to about 30 μg/kg/day.

In certain exemplary embodiments of methods of the present disclosure, the therapeutic agent that activates GPR158 administered to a patient in a dosage range of from about 0.5 ng/kg/day to about 100 ng/kg/day, from about 1 ng/kg/day to about 80 ng/kg/day, from about 3 ng/kg/day to about 50 ng/kg/day, or from about 3 ng/kg/day to about 30 ng/kg/day.

### Exemplary Antibody Activators OF GPR158

The exemplary embodiments of the present disclosure also provides compositions comprising an antibody or antibodies, as well as biologically active fragments or variants thereof, that are capable of activating GPR158 signaling through the pathway that is activated when undercarboxylated/uncarboxylated osteocalcin binds to and activates GPR158.

An antibody that activates GPR158 can be used therapeutically to treat the cognitive disorders described herein. In certain exemplary embodiments, the antibody binds to the extracellular domain of GPR158.

In certain exemplary embodiments, the antibody that activates GPR158 binds to an epitope in human GPR158 encoded by SEQ ID NO:6 or to a polypeptide having an amino acid sequence that is substantially homologous or identical to SEQ ID NO:7 or SEQ ID NO:8. In other exemplary embodiments, the antibody that activates GPR158 binds to an epitope in a polypeptide having an amino acid sequence that is at least 70%, 80%, 90%, 95%, or 99% homologous or identical to SEQ ID NO:7 or SEQ ID NO:8.

The term “epitope” refers to an antigenic determinant on an antigen to which an antibody binds. Epitopes usually consist of chemically active surface groupings of molecules such as amino acids or sugar side chains, and typically have specific three-dimensional structural characteristics, as well as specific charge characteristics. Epitopes generally have at least five contiguous amino acids but some epitopes are formed by discontiguous amino acids that are brought together by the folding of the protein that contains them.

The terms “antibody” and “antibodies” include polyclonal antibodies, monoclonal antibodies, humanized or chimeric antibodies, single chain Fv antibody fragments, Fab fragments, and F(ab′)2 fragments. Polyclonal antibodies are heterogeneous populations of antibody molecules that are specific for a particular antigen, while monoclonal antibodies are homogeneous populations of antibodies to a particular epitope contained within an antigen. Monoclonal antibodies are particularly useful in the present disclosure.

Antibody fragments that have specific binding affinity for GPR158 can be generated by known techniques. Such antibody fragments include, but are not limited to, F(ab′)2 fragments that can be produced by pepsin digestion of an antibody molecule, and Fab fragments that can be generated by reducing the disulfide bridges of F(ab′)2 fragments. Alternatively, Fab expression libraries can be constructed. See, for example, Huse et al., 1989, Science 246:1275-1281. Single chain Fv antibody fragments are formed by linking the heavy and light chain fragments of the Fv region via an amino acid bridge (e.g., 15 to 18 amino acids), resulting in a single chain polypeptide. Single chain Fv antibody fragments can be produced through standard techniques, such as those disclosed in U.S. Pat. No. 4,946,778.

Once produced, antibodies or fragments thereof can be tested for recognition of the target polypeptide by standard immunoassay methods including, for example, enzyme-linked immunosorbent assay (ELISA) or radioimmunoassay assay (RIA). See, Short Protocols in Molecular Biology eds. Ausubel et al., Green Publishing Associates and John Wiley & Sons (1992).

### Exemplary Formulation and Administration of Pharmaceutical Compositions

The exemplary embodiments of the present disclosure describes the use of the polypeptides, nucleic acids, antibodies, small molecules and other therapeutic agents described herein formulated in pharmaceutical compositions to administer to a subject. The therapeutic agents (also referred to as “active compounds”) can be incorporated into pharmaceutical compositions suitable for administration to a subject, e.g., a human. Such compositions typically comprise the polypeptides, nucleic acids, antibodies, small molecules and a pharmaceutically acceptable carrier. Preferably, e.g., such compositions are non-pyrogenic when administered to humans.

The pharmaceutical compositions of the present disclosure are administered in an amount sufficient to activate GPR158 signaling through the pathway that is activated when undercarboxylated/uncarboxylated osteocalcin binds to and activates GPR158.

As used herein the language “pharmaceutically acceptable carrier” is intended to include any and all solvents, binders, diluents, disintegrants, lubricants, dispersion media, coatings, antibacterial and antifungal agents, isotonic and absorption delaying agents, and the like, compatible with pharmaceutical administration. The use of such media and agents for pharmaceutically active substances is well known in the art. As long as any conventional media or agent is compatible with the active compound, such media can be used in the compositions of the present disclosure. Supplementary active compounds or therapeutic agents can also be incorporated into the compositions. A pharmaceutical composition of the present disclosure is formulated to be compatible with its intended route of administration. Examples of routes of administration include parenteral, e.g., intravenous, intradermal, intranasal, subcutaneous, oral, inhalation, transdermal (topical), transmucosal, and rectal administration.

The term “administer” is used in its broadest sense and includes any method of introducing the compositions of the present disclosure into a subject. This includes producing polypeptides or polynucleotides in vivo as by transcription or translation of polynucleotides that have been exogenously introduced into a subject. Thus, polypeptides or nucleic acids produced in the subject from the exogenous compositions are encompassed in the term “administer.”

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylene diamine tetra acetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Exemplary pharmaceutical compositions suitable for injectable use include sterile aqueous solutions (where the therapeutic agents are water soluble) or dispersions and sterile powders for the extemporaneous preparation of sterile injectable solutions or dispersion. For intravenous administration, suitable carriers include physiological saline, bacteriostatic water, Cremophor EL® (BASF, Parsippany, N.J.) or phosphate buffered saline (PBS). In all cases, the composition must be sterile and should be fluid to the extent that easy syringability exists. It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., undercarboxylated/uncarboxylated osteocalcin protein or an antibody that activates GPR158) in the required amount in an appropriate solvent with one or a combination of the ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yield a powder of the active ingredient plus any additional desired ingredient from a previously sterile-filtered solution thereof.

Oral compositions generally include an inert diluent or an edible carrier. They can be enclosed in gelatin capsules or compressed into tablets. Depending on the specific conditions being treated, pharmaceutical compositions of the present disclosure for treatment of cognitive disorders in mammals can be formulated and administered systemically or locally. Techniques for formulation and administration can be found in “Remington: The Science and Practice of Pharmacy” (20th edition, Gennaro (ed.) and Gennaro, Lippincott, Williams & Wilkins, 2000). For oral administration, the agent can be contained in enteric forms to survive the stomach or further coated or mixed to be released in a particular region of the GI tract by known methods. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, PRIMOGEL®, or corn starch; a lubricant such as magnesium stearate or STEROTES®; a glidant such as colloidal silicon dioxide; a sweetening agent such as sucrose or saccharin; or a flavoring agent such as peppermint, methyl salicylate, or orange flavoring.

For administration by inhalation, the compounds may be delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

If appropriate, the compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to particular cells with, e.g., monoclonal antibodies) can also be used as pharmaceutically acceptable carriers. These can be prepared according to methods known to those skilled in the art, for example, as described in U.S. Pat. No. 4,522,811.

It is especially advantageous to formulate oral or parenteral compositions in unit dosage form for ease of administration and uniformity of dosage. “Unit dosage form” as used herein refers to physically discrete units suited as unitary dosages for the subject to be treated; each unit containing a predetermined quantity of active compound calculated to produce the desired therapeutic effect in association with the required pharmaceutical carrier. The specification for the unit dosage forms of the present disclosure are dictated by and directly dependent on the unique characteristics of the active compound and the particular therapeutic effect to be achieved, and the limitations inherent in the art of compounding such an active compound for the treatment of individuals.

As indicated herein, the agent may be administered continuously by pump or frequently during the day for extended periods of time. In certain exemplary embodiments, the agent may be administered at a rate of from about 0.3-100 ng/hour, preferably about 1-75 ng/hour, more preferably about 5-50 ng/hour, and even more preferably about 10-30 ng/hour.

The agent may be administered at a rate of from about 0.1-100 μg/hr, preferably about 1-75 μg/hr, more preferably about 5-50 μg/hr, and even more preferably about 10-30 μg/hr. It will also be appreciated that the effective dosage of antibody, protein, or polypeptide used for treatment may increase or decrease over the course of a particular treatment. Changes in dosage may result and become apparent from monitoring the level of undercarboxylated/uncarboxylated osteocalcin in a biological sample, preferably blood or serum.

In an exemplary embodiment of the present disclosure, the agent can be delivered by subcutaneous, long-term, automated drug delivery using an osmotic pump to infuse a desired dose of the agent for a desired time. Insulin pumps are widely available and are used by diabetics to automatically deliver insulin over extended periods of time. Such insulin pumps can be adapted to deliver the agent for use in the methods of the present disclosure. The delivery rate of the agent can be readily adjusted through a large range to accommodate changing requirements of an individual (e.g., basal rates and bolus doses). New pumps permit a periodic dosing manner, i.e., liquid is delivered in periodic discrete doses of a small fixed volume rather than in a continuous flow manner. The overall liquid delivery rate for the device is controlled and adjusted by controlling and adjusting the dosing period. The pump can be coupled with a continuous monitoring device and remote unit, such as a system described in U.S. Pat. No. 6,560,471, entitled “Analyte Monitoring Device and Methods of Use.” In such an arrangement, the hand-held remote unit that controls the continuous blood monitoring device could wirelessly communicate with and control both the blood monitoring unit and the fluid delivery device delivering therapeutic agents for use in the methods of the present disclosure.

In some exemplary embodiments of the present disclosure, a patient is tested to determine if his serum undercarboxylated/uncarboxylated osteocalcin levels are significantly lower than normal levels (about 25% below) before administering treatment with the therapeutic agent. The frequency of administration may vary from a single dose per day to multiple doses per day. Preferred routes of administration include oral, intravenous and intraperitoneal, but other forms of administration may be chosen as well.

A “therapeutically effective amount” of a protein or polypeptide, small molecule, antibody, or nucleic acid is an amount that achieves the desired therapeutic result. For example, if a therapeutic agent is administered to treat or prevent a cognitive disorder in mammals, a therapeutically effective amount is an amount that ameliorates one or more symptoms of the disorder, or produces at least one effect selected from the group consisting of lessening of cognitive loss due to neurodegeneration associated with aging, lessening of anxiety, lessening of depression, lessening of memory loss, improving learning, and lessening of cognitive disorders associated with food deprivation during pregnancy.

A therapeutically effective amount of protein or polypeptide, small molecule or nucleic acid for use in the present disclosure typically varies and can be an amount sufficient to achieve serum therapeutic agent levels typically of between about 1 nanogram per milliliter and about 10 micrograms per milliliter in the subject, or an amount sufficient to achieve serum therapeutic agent levels of between about 1 nanogram per milliliter and about 7 micrograms per milliliter in the subject. Other preferred serum therapeutic agent levels include about 0.1 nanogram per milliliter to about 3 micrograms per milliliter, about 0.5 nanograms per milliliter to about 1 microgram per milliliter, about 1 nanogram per milliliter to about 750 nanograms per milliliter, about 5 nanograms per milliliter to about 500 nanograms per milliliter, and about 5 nanograms per milliliter to about 100 nanograms per milliliter.

The amount of therapeutic agent disclosed herein to be administered to a patient in the methods of the present disclosure can be determined by those skilled in the art through routine methods and may range from about 1 mg/kg/day to about 1,000 mg/kg/day, from about 5 mg/kg/day to about 750 mg/kg/day, from about 10 mg/kg/day to about 500 mg/kg/day, from about 25 mg/kg/day to about 250 mg/kg/day, from about 50 mg/kg/day to about 100 mg/kg/day, or other suitable amounts.

The amount of therapeutic agent disclosed herein to be administered to a patient in the methods of the present disclosure also may range from about 1 μg/kg/day to about 1,000 μg/kg/day, from about 5 μg/kg/day to about 750 μg/kg/day, from about 10 μg/kg/day to about 500 μg/kg/day, from about 25 μg/kg/day to about 250 μg/kg/day, or from about 50 μg/kg/day to about 100 μg/kg/day.

The amount of therapeutic agent disclosed herein to be administered to a patient in the methods of the present disclosure also may range from about 1 ng/kg/day to about 1,000 ng/kg/day, from about 5 ng/kg/day to about 750 ng/kg/day, from about 10 ng/kg/day to about 500 ng/kg/day, from about 25 ng/kg/day to about 250 ng/kg/day, or from about 50 ng/kg/day to about 100 ng/kg/day.

The skilled artisan will appreciate that certain factors may influence the dosage required to effectively treat a subject, including but not limited to the severity of the condition, previous treatments, the general health and/or age of the subject, and other disorders or diseases present.

Treatment of a subject with a therapeutically effective amount of a protein, polypeptide, nucleotide or antibody can include a single treatment or, preferably, can include a series of treatments.

In certain exemplary embodiments, treatment of a subject with undercarboxylated/uncarboxylated osteocalcin in order to activate GPR158 leads to undercarboxylated/uncarboxylated osteocalcin being about 10%, about 15%, about 20%, about 25%, about 30%, about 35%, about 40%, about 45%, or about 50% of the total osteocalcin in the blood of the patient.

It is understood that the appropriate dose of a small molecule agent depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, and the effect which the practitioner desires the small molecule to have. It is furthermore understood that appropriate doses of a small molecule depend upon the potency of the small molecule with respect to the expression or activity to be modulated. When one or more of these small molecules is to be administered to an animal (e.g., a human) in order to activate GPR158, a relatively low dose may be prescribed at first, with the dose subsequently increased until an appropriate response is obtained. In addition, it is understood that the specific dose level for any particular subject will depend upon a variety of factors including the activity of the specific compound employed, the age, body weight, general health, and diet of the subject, the time of administration, the route of administration, the rate of excretion, whether other drugs are being administered to the patient, and the degree of expression or activity to be modulated.

For prevention or treatment, a suitable subject can be an individual who is suspected of having, has been diagnosed as having, or is at risk of developing a cognitive disorder in mammals.

Suitable routes of administration of the pharmaceutical compositions useful in the methods of the present disclosure can include oral, intestinal, parenteral, transmucosal, transdermal, intramuscular, subcutaneous, transdermal, rectal, intramedullary, intrathecal, intravenous, intraventricular, intraatrial, intraaortal, intraarterial, or intraperitoneal administration. The pharmaceutical compositions useful in the methods of the present disclosure can be administered to the subject by a medical device, such as, but not limited to, catheters, balloons, implantable devices, biodegradable implants, prostheses, grafts, sutures, patches, shunts, or stents. In one preferred embodiment, the therapeutic agent (e.g., undercarboxylated/uncarboxylated osteocalcin) can be coated on a stent for localized administration to the target area. In this situation a slow release preparation of undercarboxylated/uncarboxylated osteocalcin, for example, is preferred.

The compounds of the present disclosure may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations and that may be consulted by those skilled in the art for techniques useful for practicing the present disclosure include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

While uncarboxylated osteocalcin crosses the blood-brain barrier, certain derivatives, variants, or modified forms of osteocalcin may not. In exemplary embodiments of the present disclosure utilizing a form of osteocalcin that does not cross the blood-brain barrier, one may take advantage of methods known in the art for transporting substances across the the blood-brain barrier. For example, the methods disclosed in U.S. Patent Application Publication No. 2013/0034590 or U.S. Patent Application Publication No.

2013/0034572 may be used. The human insulin or transferrin receptor can be utilized by targeting these receptors with a monoclonal antibody-modified osteocalcin conjugate (Pardridge, 2007, Pharm. Res. 24:1733-1744; Beduneau et al., 2008, J. Control. Release 126:44-49). Surfactant coated poly(butylcyanoacrylate) nanoparticles containing modified osteocalcin my be used (Kreuter et al., 2003, Pharm. Res. 20:409-416). Alternatively, cationic carriers such as cationic albumin conjugated to pegylated nanoparticles containing modified osteocalcin may be used to deliver modified osteocalcin to the brain (Lu et al., 2006, Cancer Res. 66:11878-11887).

The above-described methods known in the art for transporting substances across the the blood-brain barrier may also be utilized for other therapeutic agents that activate GPR158, if those other agents do not cross the blood-brain barrier on their own.

In yet another exemplary aspect of the present disclosure, undercarboxylated/uncarboxylated osteocalcin is administered as a pharmaceutical composition with a pharmaceutically acceptable excipient. Exemplary pharmaceutical compositions for undercarboxylated/uncarboxylated osteocalcin include injections as solutions or injections as injectable self-setting or self-gelling mineral polymer hybrids. Undercarboxylated/uncarboxylated osteocalcin may be administered using a porous crystalline biomimetic bioactive composition of calcium phosphate. See U.S. Pat. Nos. 5,830,682; 6,514,514; and 6,511,958 and U.S. Patent Application Publications Nos. 2006/0063699; 2006/0052327; 2003/199615; 2003/0158302; 2004/0157864; 2006/0292670; 2007/0099831 and 2006/0257492, all of which are incorporated herein in their entirety by reference.

### Exemplary Methods Of Treatment

The exemplary embdoiemnst of the present disclosure provide exemplary methods for activating the GPR158 signaling pathway for treating or preventing a variety of different cognitive disorders in mammals. According to the exemplary embdoiemnst of the present disclosure, the methods can provide an amount of an agent effective to treat or prevent a cognitive disorder associated with the GPR158 signaling pathway. The agent may be selected from the group consisting of small molecules, antibodies and nucleic acids. Such disorders include, but are not limited to, neurodegeneration associated with aging, anxiety, depression, memory loss, and cognitive disorders associated with food deprivation during pregnancy.

In certain exemplary embodiments, the methods can comprise identifying a patient in need of treatment or prevention of neurodegeneration associated with aging, anxiety, depression, memory loss, learning difficulties, or cognitive disorders associated with food deprivation during pregnancy and then applying the methods disclosed herein to the patient.

In one exemplary embodiment of the present disclosure, the method of treatment comprises administering to a patient in need thereof a therapeutically effective amount of undercarboxylated/uncarboxylated osteocalcin sufficient to raise the patient's blood level of undercarboxylated/uncarboxylated osteocalcin compared to the pretreatment patient level. Since undercarboxylated/uncarboxylated osteocalcin can cross the blood/brain barrier, this can lead to therapeutically effective levels of undercarboxylated/uncarboxylated osteocalcin in target areas of the brain that express GPR158. Preferably, the patient is a human. In another embodiment, the method of treatment comprises administering to a patient in need thereof a therapeutically effective amount of undercarboxylated/uncarboxylated osteocalcin sufficient to raise the ratio of undercarboxylated/uncarboxylated osteocalcin to total osteocalcin in the patient's blood compared to the pretreatment patient ratio.

In another exemplary aspect of the present disclosure, a method is provided for treating or preventing a cognitive disorder in a mammal comprising administering to a mammal in need thereof undercarboxylated/uncarboxylated osteocalcin in a therapeutically effective amount, sufficient to activate GPR158, and that produces at least one effect selected from the group consisting of lessening of cognitive loss due to neurodegeneration associated with aging, lessening of anxiety, lessening of depression, lessening of memory loss, improving learning, and lessening of cognitive disorders associated with food deprivation during pregnancy, compared to pretreatment levels. Preferably, the mammal is a human.

Certain exemplary embodiments of the present disclosure is directed to methods (i) for treating or preventing a cognitive disorder in a mammal comprising administering to a mammal in need of such treatment or prevention in a therapeutically effective amount an agent that activates GPR158 to a degree sufficient to produce at least one effect selected from the group consisting of lessening of cognitive loss due to neurodegeneration associated with aging, lessening of anxiety, lessening of depression, lessening of memory loss, improving learning, and lessening of cognitive disorders associated with food deprivation during pregnancy, compared to pretreatment levels. Preferably, the mammal is a human.

In the exemplary methods described herein, it will be understood that “treating” a disease or disorder encompasses not only improving the disease or disorder or its symptoms but also retarding the progression of the disease or disorder or ameliorating the deleterious effects of the disease or disorder.

Efficacy of the methods of treatment described herein can be monitored by determining whether the methods ameliorate any of the symptoms of the disease or disorder being treated.

### Exemplary Drug Screening and Assays

Cell-based and non-cell based methods of drug screening are provided to identify candidate agents that are capable of activating GPR158 signaling through the pathway that is activated when undercarboxylated/uncarboxylated osteocalcin activates GPR158. Such agents find use in treating or preventing cognitive disorders in mammals.

Non-cell based screening methods are provided to identify compounds that bind to and activate GPR158. Such non-cell based methods include a method to identify, or assay for, an agent that binds to GPR158, the method comprising the steps of: (i) providing a mixture comprising GPR158 or a fragment or variant thereof, (ii) contacting the mixture with a candidate agent, (iii) determining whether the candidate agent binds to the GPR158 or a fragment or variant thereof in the mixture, wherein if the agent binds to the GPR158 or a fragment or variant thereof. The method optionally comprises (iv) determining whether the agent activates GPR158 and/or (v) administering the agent to a patient in need of treatment for a cognitive disorder in mammals. In certain exemplary embodiments, the mixture comprises membrane fragments comprising GPR158 or a fragment or variant thereof

The binding of the agent to the target molecule in the above-described assay may be determined through the use of competitive binding assays. The competitor is a binding moiety known to bind to GPR158 or a fragment or variant thereof. Under certain circumstances, there may be competitive binding as between the agent and the binding moiety, with the binding moiety displacing the agent or the agent displacing the binding moiety. In certain exemplary embodiments, the competitor is undercarboxylated/uncarboxylated osteocalcin.

Either the agent or the competitor may be labeled. Either the agent, or the competitor is added first to the GPR158 or a fragment or variant thereof for a time sufficient to allow binding. Incubations may be performed at any temperature which facilitates optimal binding, typically between 4° C. and 40° C. Incubation periods may also be chosen for optimum binding, but may also optimized to facilitate rapid high throughput screening. Typically, between 0.1 and 1 hour will be sufficient. Excess agent and competitor are generally removed or washed away.

Using such assays, the competitor may be added first, followed by the agent. Displacement of the competitor is an indication that the agent is binding to the GPR158 or a fragment or variant thereof and thus may be capable of modulating the activity of the GPR158. In this embodiment, either component can be labeled. Thus, for example, if the competitor is labeled, the presence of label in the wash solution indicates displacement by the agent.

In another example, the agent is added first, with incubation and washing, followed by the competitor. The absence of binding by the competitor may indicate that the agent is bound to the GPR158 or a fragment or variant thereof with a higher affinity than the competitor. Thus, if the agent is labeled, the presence of the label on the GPR158 or a fragment or variant thereof, coupled with a lack of competitor binding, may indicate that the agent is capable of binding to the GPR158 or a fragment or variant thereof

The exemplary method may comprise differential screening to identify agents that are capable of activating GPR158. In such an instance, the exemplary methods can comprise combining GPR158 or a fragment or variant thereof and a competitor in a first sample. A second sample comprises an agent, the GPR158 or a fragment or variant thereof, and a competitor. Addition of the agent is performed under conditions which allow the modulation of the activity of the GPR158 or a fragment or variant thereof. The binding of the competitor is determined for both samples, and a change, or difference in binding between the two samples indicates the presence of an agent capable of binding to the GPR158 or a fragment or variant thereof and potentially activating the activity of GPR158. That is, if the binding of the competitor is different in the second sample relative to the first sample, the agent is capable of binding to the GPR158 or a fragment or variant thereof

Positive controls and negative controls may be used in the assays. Preferably, all control and test samples are performed in at least triplicate to obtain statistically significant results. Incubation of all samples is for a time sufficient for the binding of the agent to the GPR158 or a fragment or variant thereof. Following incubation, all samples are washed free of non-specifically bound material and the amount of bound, generally labeled agent determined. For example, where a radiolabel is employed, the samples may be counted in a scintillation counter to determine the amount of bound agent.

A variety of other reagents may be included in the screening assays. These include reagents like salts, neutral proteins, e.g. albumin, detergents, etc. which may be used to facilitate optimal protein-protein binding and/or reduce non-specific or background interactions. Also, reagents that otherwise improve the efficiency of the assay, such as protease inhibitors, nuclease inhibitors, anti-microbial agents, etc., may be used. The mixture of components may be added in any order that provides for the requisite binding.

Thus, in one example, the methods comprise combining a sample comprising GPR158 activity. By GPR158 activity is meant one or more of the biological activities associated with the activation of GPR158 by osteocalcin. The screening assays are designed to find agents that are useful in the treatment of cognitive disorders in mammals.

The agents identified by the methods described above may be further screened to identify those agents that activate GPR158 but do not activate. In certain exemplary embodiments, the further screening may comprise:

(a) providing a cell that expresses GPRC6A;

(b) exposing the cell to an agent that has been identified as an activator of GPR158; and

(c) determining that the candidate substance does not bind to and/or activate the GPRC6A expressed by the cell.

Optionally, the method also comprises: (d) determining if the agent that has been identified as an activator of GPR158is suitable for use in the prevention and treatment of a cognitive disorder in mammals.

In certain exemplary embodiments, step (a) can comprise providing cells that recombinantly express GPRC6A. In certain exemplary embodiments, the cells that recombinantly express GPRC6A are NIH 3T3 cells, HEK 293 cells, BHK cells, COS cells, CHO cells, Xenopus oocytes, or insect cells. In certain exemplary embodiments, the GPRC6A is human GPRC6A. In certain exemplary embodiments, the GPRC6A is the protein disclosed at GenBank accession no. AF502962.

In certain exemplary embodiments, the agent that has been identified as an activator of GPR158 is from a library of candidate substances. In certain exemplary embodiments, the entire library of substances is screened to identify agents that activate GPR158. In certain exemplary embodiments, a portion of the library is screened.

In certain exemplary embodiments, step (b) is carried out by growing the cell in tissue culture and adding the agent that has been identified as an activator of GPR158 to the medium in which the cell is growing or has been grown. Alternatively, the medium in which the cell is growing or has been grown may be removed and fresh medium containing the agent that has been identified as an activator of GPR158 may be added the tissue culture plate or well in which the cell is growing or has been grown.

In certain exemplary embodiments, step (c) comprises determining if the agent that has been identified as an activator of GPR158 competes with labeled uncarboxlated osteocalcin for binding to the GPRC6A. In certain exemplary embodiments, step (c) comprises labeling the agent that has been identified as an activator of GPR158 and determining if the labeled agent that has been identified as an activator of GPR158 binds to the GPRC6A expressed by the cell.

In certain exemplary embodiments, step (c) comprises determining if the agent that has been identified as an activator of GPR158 produces a physiological response in the cell selected from the group consisting of: an increase in the concentration of cAMP in the cell. an increase in testosterone synthesis in the cell, an increase in the expression of StAR in the cell, an increase in the expression of Cypl1a in the cell, an increase in the expression of Cyp17 in the cell, an increase in the expression of 3β-HSD in the cell, an increase in the expression of Grth in the cell, an increase in the expression of tACE in the cell, an increase in CREB phosphorylation in the cell, and a decrease in the amount cleaved Caspase 3 in the cell. The physiological response may also be a combination of any of the foregoing physiological responses. In certain exemplary embodiments, the physiological response is an increase in the concentration of cAMP in the cell together with a lack of an increase in tyrosine phosphorylation, ERK activation, and intracellular calcium accumulation. In exemplary embodiments where a physiological response is determined, it may be advantageous to use a cell that does not naturally express GPRC6A but that has been engineered to recombinantly express GPRC6A. In such cases, the cell prior to transformation to a state that recombinantly expresses GPRC6A can serve as a negative control.

In certain exemplary embodiments, step (c) can comprise determining if the agent that has been identified as an activator of GPR158 affects the binding of a G protein to the GPRC6A. Here, too, it may be advantageous to use cells that recombinantly express GPRC6A and to use those same cells before transformation as negative controls. In certain exemplary embodiments, the cell is co-transfected with a construct encoding GPRC6A and a construct encoding a Ga protein. See, e.g., Christiansen et al., 2007, Br. J. Pharmacol. 150:798-807 and Pi et al., 2005, J. Biol. Chem. 280:40201-40209.

Exemplary embodiments of the present disclosure also provide cell-based screening methods to identify agents that activate GPR158 and are suitable for use in the prevention and treatment of a cognitive disorder in mammals where the methods comprise:

(a) providing a cell containing GPR158 protein;

(b) exposing the cell to a candidate agent;

(c) determining that the candidate agent activates the GPR158 in the cell; and

(d) determining if the candidate agent is suitable for use in the prevention and treatment of a cognitive disorder in mammals.

In certain exemplary embodiments, step (a) can comprise providing a cell that recombinantly expresses GPR158. In certain exemplary embodiments, the cells that recombinantly express GPR158 are NIH 3T3 cells, HEK 293 cells, BHK cells, COS cells, CHO cells, Xenopus oocytes, or insect cells. In certain exemplary embodiments, the GPR158 is encoded by the nucleotide sequence shown in SEQ ID NO: 6. In certain exemplary embodiments, the GPR158 comprises the amino acid sequence shown in SEQ ID NO: 7 or SEQ ID NO:8.

In certain exemplary embodiments, the candidate agent can be from a library of candidate agents. In certain exemplary embodiments, the entire library of agents is exposed to the cell. In certain exemplary embodiments, a portion of the library is exposed to the cell.

In certain exemplary embodiments, step (c) can comprise determining if the candidate agent competes with labeled uncarboxlated osteocalcin for binding to the GPR158.

In certain exemplary embodiments, step (c) comprises labeling the candidate agent and determining if the labeled candidate agent binds to the GPR158 in the cell.

In certain exemplary embodiments, step (d) can comprise administering the candidate agent to a mammal and determining that the candidate agent produces an effect in the mammal selected from the group consisting of lessening of cognitive loss due to neurodegeneration associated with aging, lessening of anxiety, lessening of depression, lessening of memory loss, improving learning, and lessening of cognitive disorders associated with food deprivation during pregnancy.

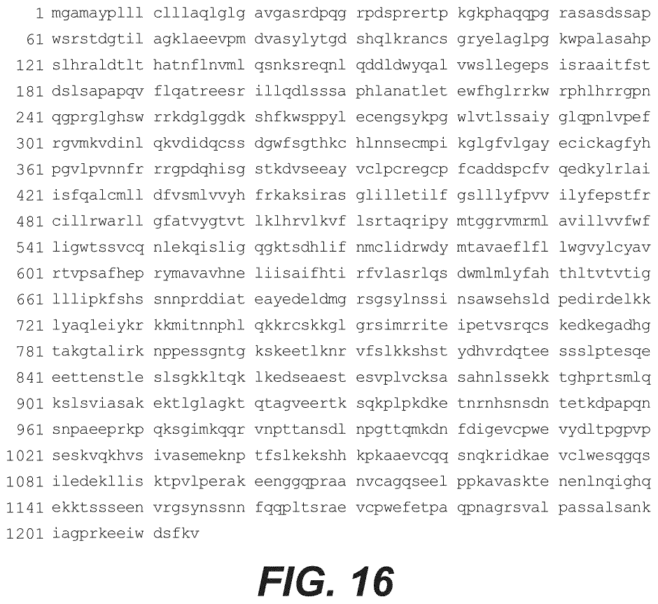

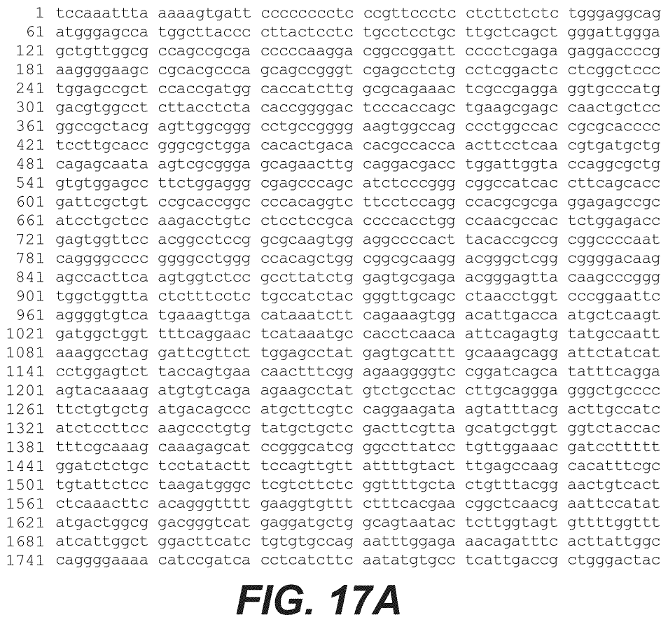

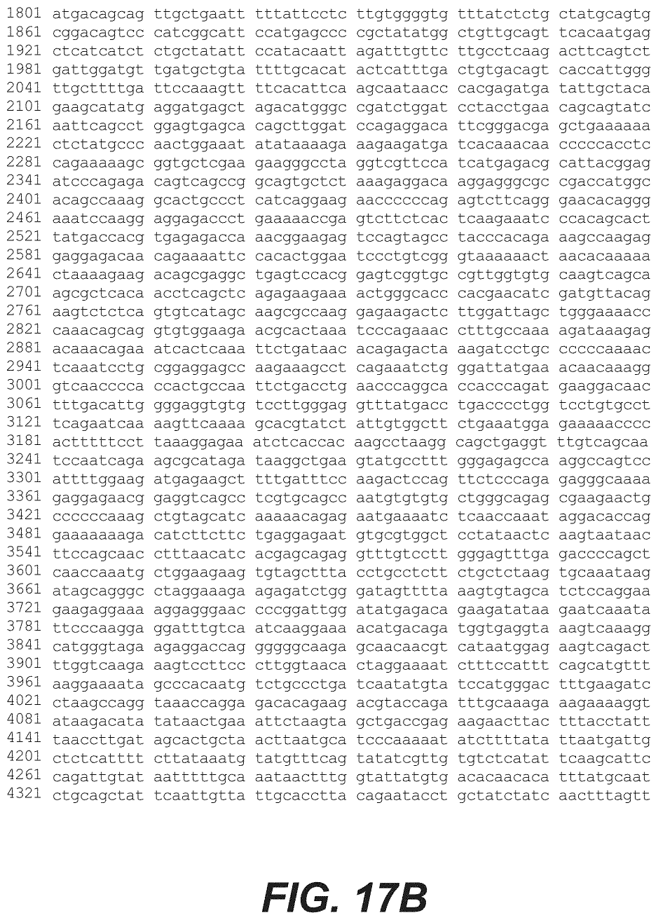

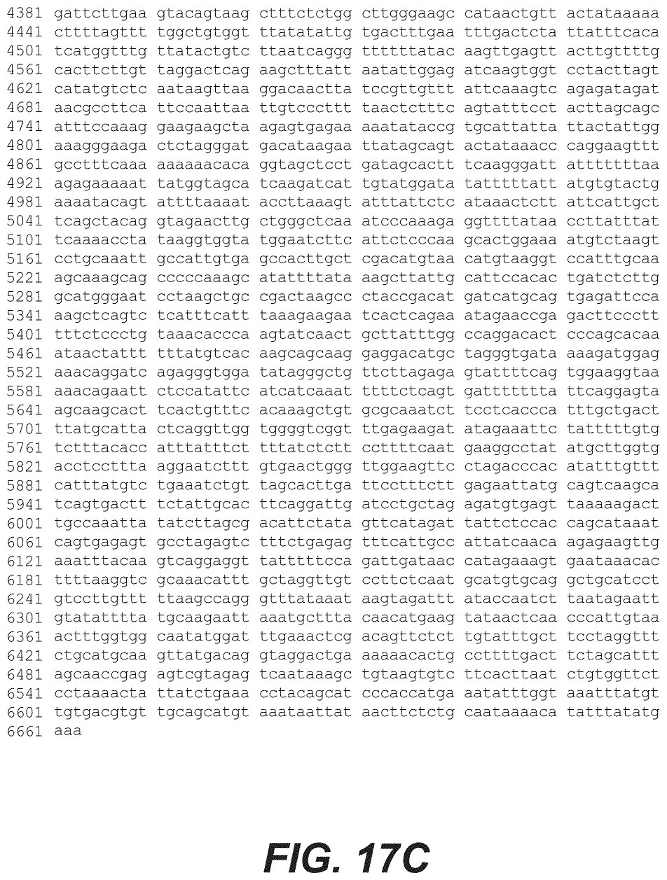

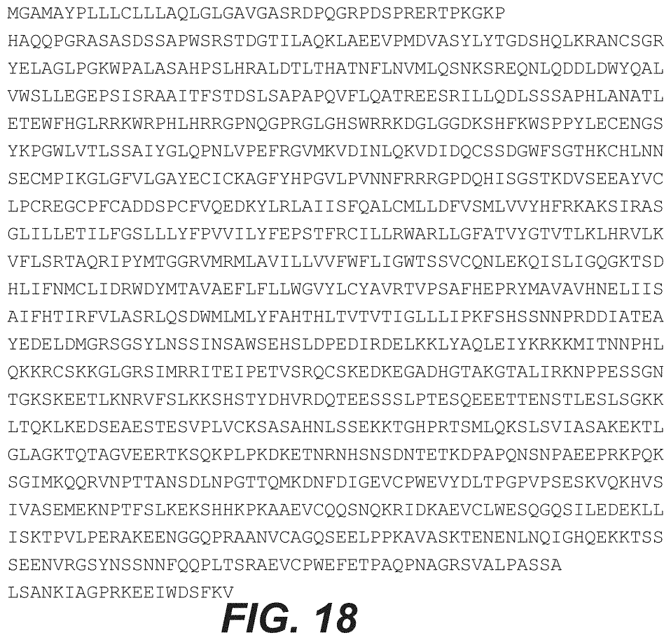

In certain exemplary embodiments of the methods described herein, GPR158 is the protein disclosed at NCBI reference sequence NP 065803.2 or NM 020752.2. The nucleotide and amino acid sequences disclosed at NCBI reference sequence NP 065803.2 or NM 020752.2 are shown in FIGS. 16, 17A-C, and 18 herein, respectively.

In certain exemplary embodiments of the methods disclosed above, GPR158 is a protein homologous to the protein disclosed at NCBI reference sequence NP 065803.2 or NM 020752.2. In certain exemplary embodiments of the methods described herein, GPR158 is a protein having about 80-99%, about 85-97%, or about 90-95% amino acid sequence identity to the protein disclosed at NCBI reference sequence NP 065803.2 or NM 020752.2.

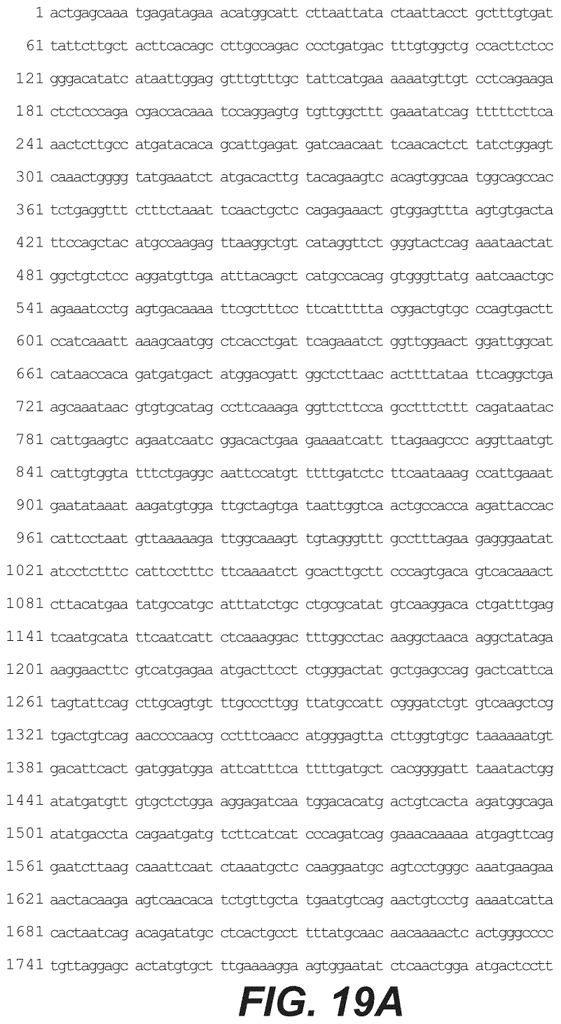

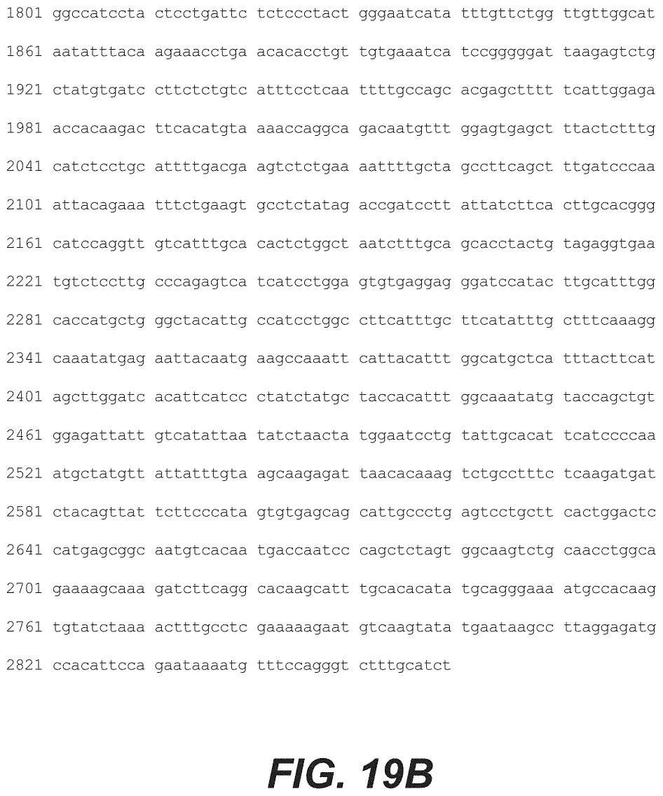

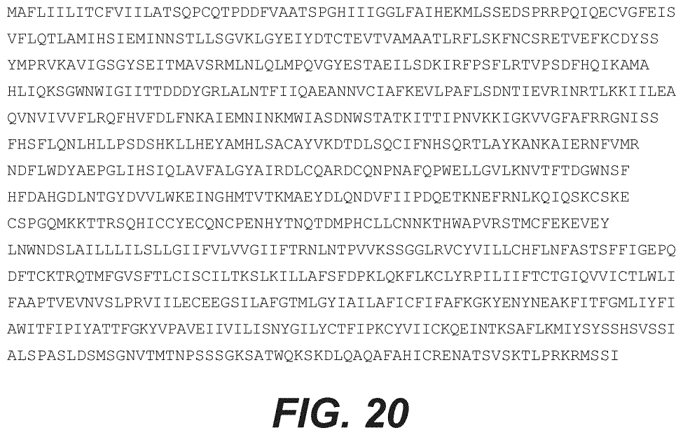

In certain exemplary embodiments of the methods described herein, GPRC6A is the protein disclosed at GenBank accession no. AF502962. The nucleotide and amino acid sequences disclosed at GenBank accession no. AF502962 are shown in FIGS. 19A-B and 20 herein, respectively.

In certain exemplary embodiments of the methods described herein, GPRC6A is a protein homologous to the protein disclosed at GenBank accession no. AF502962. In certain exemplary embodiments of the methods disclosed above, GPRC6A is a protein having about 80-99%, about 85-97%, or about 90-95% amino acid sequence identity to the protein disclosed at GenBank accession no. AF502962.

In certain exemplary embodiments of the methods described herein, GPRC6A is the protein disclosed Wellendorph & Branner-Osborne, 2004, Gene 335:37-46.

In certain exemplary embodiments of the present disclosure, the agents identified by the methods of screening against GPR158 and/or GPRC6A are administered to a mammal in need of treatment for a cognitive disorder. Accordingly, the present disclosure includes a method of treating cognitive disorders in mammals comprising administering to a mammal in need of treatment for a cognitive disorder a pharmaceutical composition comprising a therapeutically effective amount of an agent that activates GPR158 but does not activate GPRC6A and a pharmaceutically acceptable carrier or excipient.

Agents that activate GPCR6A include ornithine, lysine, and arginine and may be used as control in the above-described assays (Christiansen et al., 2007, Br. J. Pharmacol. 150:798-807).

Cells to be used in the screening or assaying methods described herein include cells that naturally express GPR158 as well as cells that have been genetically engineered to express (or overexpress) GPR158.

The term “agent” as used herein includes any molecule, e.g., protein, oligopeptide, small organic molecule, polysaccharide, polynucleotide, lipid, etc., or mixtures thereof

Generally, in the assays described herein, a plurality of assay mixtures is run in parallel with different agent concentrations to obtain a differential response to the various concentrations. Typically, one of these concentrations serves as a negative control, i.e., is at zero concentration or below the level of detection.

Agents for use in screening encompass numerous chemical classes, though typically they are organic molecules, preferably small organic compounds having a molecular weight of more than 100 and less than about 2,500 daltons, preferably less than about 500 daltons. Agents comprise functional groups necessary for structural interaction with proteins, particularly hydrogen bonding, and typically include at least an amine, carbonyl, hydroxyl or carboxyl group, preferably at least two of these functional chemical groups. The agents often comprise cyclical carbon or heterocyclic structures and/or aromatic or polyaromatic structures substituted with one or more of the above functional groups. Agents are also found among biomolecules including peptides, saccharides, fatty acids, steroids, purines, pyrimidines, derivatives, structural analogs or combinations thereof. Particularly preferred biomolecules are peptides.

Libraries of high-purity small organic ligands and peptides that have well-documented pharmacological activities are available from numerous sources for use in the assays herein. One example is an NCI diversity set which contains 1,866 drug-like compounds (small, intermediate hydrophobicity). Another is an Institute of Chemistry and Cell Biology (ICCB; maintained by Harvard Medical School) set of known bioactives (467 compounds) which includes many extended, flexible compounds. Some other examples of the ICCB libraries are: Chem Bridge DiverSet E (16,320 compounds); Bionet 1 (4,800 compounds); CEREP (4,800 compounds); Maybridge 1 (8,800 compounds); Maybridge 2 (704 compounds); Maybridge HitFinder (14,379 compounds); Peakdale 1 (2,816 compounds); Peakdale 2 (352 compounds); ChemDiv Combilab and International (28,864 compounds); Mixed Commercial Plate 1 (352 compounds); Mixed Commercial Plate 2 (320 compounds); Mixed Commercial Plate 3 (251 compounds); Mixed Commercial Plate 4 (331 compounds); ChemBridge Microformat (50,000 compounds); Commercial Diversity Setl (5,056 compounds). Other NCI Collections are: Structural Diversity Set, version 2 (1,900 compounds); Mechanistic Diversity Set (879 compounds); Open Collection 1 (90,000 compounds); Open Collection 2 (10,240 compounds); Known Bioactives Collections: NINDS Custom Collection (1,040 compounds); ICCB Bioactives 1 (489 compounds); SpecPlus Collection (960 compounds); ICCB Discretes Collections. The following ICCB compounds were collected individually from chemists at the ICCB, Harvard, and other collaborating institutions: ICCB1 (190 compounds); ICCB2 (352 compounds); ICCB3 (352 compounds); ICCB4 (352 compounds). Natural Product Extracts: NCI Marine Extracts (352 wells); Organic fractions—NCI Plant and Fungal Extracts (1,408 wells); Philippines Plant Extracts 1 (200 wells); ICCB-ICG Diversity Oriented Synthesis (DOS) Collections; DDS1 (DOS Diversity Set) (9600 wells). Compound libraries are also available from commercial suppliers, such as ActiMol, Albany Molecular, Bachem, Sigma-Aldrich, TimTec, and others.

Known and novel pharmacological agents identified in screens may be further subjected to directed or random chemical modifications, such as acylation, alkylation, esterification, or amidification to produce structural analogs.

When screening, designing, or modifying compounds, other factors to consider include the Lipinski rule-of-five (not more than 5 hydrogen bond donors (OH and NH groups); not more than 10 hydrogen bond acceptors (notably N and O); molecular weight under 500 g/mol; partition coefficient log P less than 5), and Veber criteria, which are recognized in the pharmaceutical art and relate to properties and structural features that make molecules more or less drug-like.

The agent may be a protein. By “protein” in this context is meant at least two covalently attached amino acids, and includes proteins, polypeptides, oligopeptides and peptides. The protein may be made up of naturally occurring amino acids and peptide bonds, or synthetic peptidomimetic structures. Thus “amino acid,” or “peptide residue,” as used herein means both naturally occurring and synthetic amino acids. For example, homo-phenylalanine, citrulline and norleucine are considered amino acids for the purposes of the present disclosure. “Amino acids” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations.

The agent may be a naturally occurring protein or fragment or variant of a naturally occurring protein. Thus, for example, cellular extracts containing proteins, or random or directed digests of proteinaceous cellular extracts, may be used. In this way, libraries of prokaryotic and eukaryotic proteins may be made for screening against one of the various proteins. Libraries of bacterial, fungal, viral, and mammalian proteins, with the latter being preferred, and human proteins being especially preferred, may be used.

Agents may be peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “random” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized agent bioactive proteinaceous agents.

The library may be fully randomized, with no sequence preferences or constants at any position. Alternatively, the library may be biased. That is, some positions within the sequence are either held constant, or are selected from a limited number of possibilities. For example, the nucleotides or amino acid residues are randomized within a defined class, for example, of hydrophobic amino acids, hydrophilic residues, sterically biased (either small or large) residues, towards the creation of cysteines, for cross-linking, prolines for SH3 domains, serines, threonines, tyrosines or histidines for phosphorylation sites, etc., or to purines, etc.

The agent may be an isolated nucleic acid or oligonucleotide. By “nucleic acid” or “oligonucleotide” or grammatical equivalents herein means at least two nucleotides covalently linked together. Such nucleic acids will generally contain phosphodiester bonds, although in some cases, as outlined below, nucleic acid analogs are included that may have alternate backbones, comprising, for example, phosphoramide (Beaucage et al., 1993, Tetrahedron 49:1925 and references therein; Letsinger, 1970, J. Org. Chem. 35:3800; Sprinzl et al., 1977, Eur. J. Biochem. 81:579; Letsinger et al., 1986, Nucl. Acids Res. 14:3487; Sawai et al, 1984, Chem. Lett. 805; Letsinger et al., 1988, J. Am. Chem. Soc. 110:4470; and Pauwels et al., 1986, Chemica Scripta 26:141); phosphorothioate (Mag et al., 1991, Nucleic Acids Res. 19:1437; and U.S. Patent No. 5,644,048), phosphorodithioate (Briu et al., 1989, J. Am. Chem. Soc. 111:2321); O-methylphophoroamidite linkages (see Eckstein, Oligonucleotides and Analogues: A Practical Approach, Oxford University Press), and peptide nucleic acid backbones and linkages (see Egholm, 1992, J. Am. Chem. Soc. 114:1895; Meier et al., 1992, Chem. Int. Ed. Engl. 31:1008; Nielsen, 1993, Nature, 365:566; Carlsson et al., 1996, Nature 380:207); all of which publications are incorporated by reference and may be consulted by those skilled in the art for guidance in designing nucleic acid agents for use in the methods described herein.

Other analog nucleic acids include those with positive backbones (Denpcy et al., 1995, Proc. Natl. Acad. Sci. USA 92:6097); non-ionic backbones (U.S. Pat. Nos. 5,386,023; 5,637,684; 5,602,240; 5,216,141; and 4,469,863; Kiedrowshi et al., 1991, Angew. Chem. Intl. Ed. English 30:423; Letsinger et al., 1988, J. Am. Chem. Soc. 110:4470;

Letsinger et al., 1994, Nucleoside & Nucleoside 13:1597; Chapters 2 and 3, ASC Symposium Series 580, “Carbohydrate Modifications in Antisense Research,” Ed. Y. S. Sanghui and P. Dan Cook; Mesmaeker et al., 1994, Bioorganic & Medicinal Chem. Lett. 4:395; Jeffs et al., 1994, J. Biomolecular NMR 34:17); and non-ribose backbones, including those described in U.S. Pat. Nos. 5,235,033 and 5,034,506, and Chapters 6 and 7, ASC Symposium Series 580, “Carbohydrate Modifications in antisense Research,” Ed. Y. S. Sanghui and P. Dan Cook. Nucleic acids containing one or more carbocyclic sugars are also included within the definition of nucleic acids that may be used as agents as described herein. Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments. In addition, mixtures of naturally occurring acids and analogs can be made. Alternatively, mixtures of different nucleic acid analogs, and mixtures of naturally occurring nucleic acids and analogs may be made. The nucleic acids may be single stranded or double stranded, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xanthine hypoxanthine, isocytosine, isoguanine, etc.

As described above generally for proteins, nucleic acid agents may be naturally occurring nucleic acids, random nucleic acids, or “biased” random nucleic acids. For example, digests of prokaryotic or eukaryotic genomes may be used as outlined above for proteins.

The agents may be obtained from combinatorial chemical libraries, a wide variety of which are available commercially or in the literature. By “combinatorial chemical library” herein is meant a collection of diverse chemical compounds generated in a defined or random manner, generally by chemical synthesis. Millions of chemical compounds can be synthesized through combinatorial mixing.

The determination of the binding of the agent to GPR158 or GPRC6A may be done in a number of exemplary ways. In a preferred exemplary embodiment, the agent is labeled, and binding determined directly. For example, this may be done by attaching all or a portion of GPR158 or GPRC6A to a solid support, adding a labeled agent (for example an agent comprising a radioactive or fluorescent label), washing off excess reagent, and determining whether the label is present on the solid support. Various blocking and washing steps may be utilized as is known in the art.

By “labeled” herein is meant that the agent is either directly or indirectly labeled with a label which provides a detectable signal, e.g. a radioisotope (such as 3H, 14C, 32P, 33P, 35S, or 125I), a fluorescent or chemiluminescent compound (such as fluorescein isothiocyanate, rhodamine, or luciferin), an enzyme (such as alkaline phosphatase, beta-galactosidase or horseradish peroxidase), antibodies, particles such as magnetic particles, or specific binding molecules, etc. Specific binding molecules include pairs, such as biotin and streptavidin, digoxin and antidigoxin, etc. For the specific binding members, the complementary member would normally be labeled with a molecule which provides for detection, in accordance with known procedures, as outlined above. The label can directly or indirectly provide a detectable signal. Only one of the components may be labeled. Alternatively, more than one component may be labeled with different labels.

Transgenic mice, including knock in and knock out mice, and isolated cells from them that over or under express the nucleic acids disclosed herein (e.g., cDNA for GPR158 or GPRC6A) can be made using routine methods known in the art. In certain instances, nucleic acids are inserted into the genome of the host organism operably connected to and under the control of a promoter and regulatory elements (endogenous or heterogeneous) that will cause the organism to over express the nucleic acid gene or mRNA. One example of an exogenous/heterogeneous promoter included in the transfecting vector carrying the gene to be amplified is alpha 1(I) collagen. Many such promoters are known in the art.

Disclosed herein are transgenic mice and mouse cells, and transfected human cells overexpressing GPR158 or GPRC6A. Also disclosed herein are mutant mice that have deletions of one or both alleles for GPR158 and/or GPRC6A, and various combinations of mutants.

Also disclosed herein are vectors carrying the cDNA or mRNA encoding GPR158 or GPRC6A for insertion into the genome of a target animal or cell. Such vectors can optionally include promoters and regulatory elements operably linked to the cDNA or mRNA. By “operably linked” is meant that promoters and regulatory elements are connected to the cDNA or mRNA in such a way as to permit expression of the cDNA or mRNA under the control of the promoters and regulatory elements.

The present disclosure is illustrated herein by the following examples, which should not be construed as limiting. The contents of all references, pending patent applications and published patents, cited throughout this application are hereby expressly incorporated by reference. Those skilled in the art will understand that this disclosure may be embodied in many different forms and should not be construed as limited to the exemplary embodiments set forth herein. Rather, these exemplary embodiments are provided so that this disclosure will fully convey the present disclosure to those skilled in the art. Many modifications and other exemplary embodiments of the present disclosure will come to mind in one skilled in the art to which this disclosure pertains having the benefit of the teachings presented in the foregoing description. Although specific terms are employed, they are used as in the art unless otherwise indicated.

### EXAMPLES

### Example 1

### Materials and methods

In Vivo Experiments

Osteocalcin−/−, Gprc6a−/−, Osteocalcin-mCherry, and Osteocalcin foxed mice have been previously described (Ducy et al., 1996, Nature 382:448-452; Oury et al., 2011, Cell 144:796-809). Mouse genotypes were determined by PCR. For all experiments, controls were littermate female WT, Cre-expressing, or flox/flox. All mice were maintained on a pure 129-Sv genetic background except for the inducible deletion Osteocalcin model (mix background: 25% C57/BL6 and 75% 129sv). For inducible gene deletion mice, tamoxifen was prepared in corn oil and injected intraperitoneally (IP) (1 mg/20g of body weight) over one week. For osteocalcin delivery to pregnant mice, IP injections (240 ng/day) were performed as soon as a plug was present daily until delivery (E0.5-E18.5). For osteocalcin or leptin infusion in adult Osteocalcin−/− or ob/ob mice, pumps (Alzet micro-osmotic pump, Model 1002) delivering osteocalcin (300 ng/hr), leptin (50 ng/hr), or vehicle were surgically installed subcutaneously in the backs of 3-month old mice. For the postnatal rescue of cognitive functions in Osteocalcin−/− mice, osteocalcin (10 ng/hour) or vehicle were delivered intrasubventricularly (icv) as previously described (Ducy et al., 2000, Cell 100:197-207). Leptin and osteocalcin content in sera and tissues were determined by ELISA.

Maternal-fetal transport of osteocalcin was monitored using ex vivo dual perfusion of the mouse placenta (Goeden & Bonnin, 2013, Nature Protocols 8:66-74). Osteocalcin (300 ng/ml) was injected on the maternal side through the uterine artery in placentas obtained from WT mice at E14.5, E15.5, and E18.5 of pregnancy (n=3 independent perfusions per age). Osteocalcin transport through the placenta was analyzed by measuring the concentration of osteocalcin present in fetal eluates obtained through the umbilical vein, each time point (1-9) corresponding to a 10 minute collection period (at 5 μl/min). Collection time points (from 10 to 30 min of perfusion) were obtained during materal fluid infusion; time points 4-6 (from 30 to 60 min of perfusion) were obtained during osteocalcin infusion into the maternal uterine artery, whereas for time points 7-9 (from 60 to 90 min of perfusion, respectively) the maternal uterine artery was infused with maternal fluid alone.

Histology

All dissections were performed in ice-cold PBS 1× under a Leica MZ8 dissecting light microscope. Brainstems were isolated from the cerebellum and the hypothalamus was removed from the midbrain during collection. All parts of the brain isolated were flash frozen in liquid nitrogen and kept at -80° C. until use.

Immunofluorescence of whole adult and embryonic brains was performed on 20 μm coronal cryostat slices of tissue fixed with 4% PFA, embedded in cryomatrix (Tissue-Tek) and stored at −80° C. Sections were allowed to dry at room temperature, post-fixed in 4% PFA followed by permeabilization with 0.1% Triton detergent. After room temperature blocking with donkey serum, sections were incubated with anti 5-HT (Sigma) or anti-Neun antibody (Millipore) overnight at 4° C. Anti-5-HT slides were further incubated with donkey anti-rabbit cy-3 conjugated antibody (Jackson Laboratories). Slides were mounted with Fluorogel (Electron Microscopy Sciences).

For in situ mRNA hybridization, 20 μM coronal and sagittal sections of adult mouse brain were cryostat sectioned and collected on positively charged microscope slides. Cryosections were incubated with a DIG-labeled probe at 69° C. followed by incubation with alkaline phosphatase-conjugated anti-DIG antibody, and developed by incubation with NBT/BCIP.

Cresyl violet staining to visualize brain morphology was carried out by incubating 20 μm cryosections defatted with 1:1 chloroform:ethanol in cresyl violet acetate (1 g/L) overnight. The stain was differentiated using ethanol and xylene and mounted using DPX mounting medium for histology (Sigma).

To assess apoptosis in WT and Osteocalcin−/− brains, 20 μm cryostat sections were processed using theAPOPTAG® Fluorescein Direct In Situ Apoptosis Detection Kit (Millipore) according to manufacturer's protocol. Images were obtained using Leica DM 4000B, and Image J was used to quantify cell number and intensity of staining.

Binding Assays

Brains from 8-week-old mice were snap-frozen in isopentane, and 20mm thick sections were prepared and desiccated overnight at 4° C. under vacuum. On the following day, sections were rehydrated in ice-cold binding buffer (50 mM TrisHCl [pH 7.4], 10 mM MgCl₂, 0.1 mM EDTA and 0.1% BSA) for 15 min and incubated for 1 hr in the presence of biotinylated osteocalcin (3, 30, 300, 3000 ng/ml) or biotynylated recombinant GST as a control (10 μg/ml). After washing in harvesting buffer (50 mM Tris-HCl, pH 7.4), samples were fixed in 4% paraformaldehyde for 15 min, washed in PBS and incubated with goat anti-biotin antibody (1:1000, Vector laboratories) over night at 4° C. Signal was visualized by incubating with anti-goat IgG Cy-3 using Leica DM 5000B microscope (Leica). The binding assays were perform on adjacent sections for each conditions tested.

Biochemistry and Molecular Biology

For western blotting, frozen hippocampi from E18.5 embryos were lysed and homogenized in 250 μl tissue lysis buffer (25 mM Tris HCl 7.5; 100 mM NaF; 10 mM Na4P2O7; 10 mmM EDTA; 1% NP 40). Samples were pooled together in threes by genotype to reduce variability. Proteins were transferred to nitrocellulose membranes and blocked with TBST-5% milk prior to overnight incubation with primary antibody in TBST-5% BSA. HRP-coupled secondary antibodies and ECL were used to visualize the signal.

For gene expression studies, RNA was isolated from primary neurons or tissue using TRIZOL® (Invitrogen). cDNA synthesis was performed following a standard protocol from Invitrogen and qPCR analyses were done using specific quantitative PCR primers from SABiosciences (http://www.sabiosciences.com/RT2PCR.php).

Serotonin, dopamine, norepinephrine, and GABA contents were measured by HPLC as previously described (Bach et al., 2011, J. Neurochem. 118:1067-1074). Neurotransmitter contents in 7 to 15 mice of each genotype were measured in cerebral cortex, striatum, hippocampus, hypothalamus, midbrain, brainstem, and cerebellum.

Cell Biology

For primary culture of hindbrain neurons, E14.5 embryos were obtained from matings of 129-Sv WT mice. Hindbrains were dissected out and collected in ice-cold filter sterilized HBSS buffered with 10 mM HEPES until dissection was complete, at which point they were finely chopped into 2 mm cubes, dissociated by trituration with a fire-polished Pasteur pipette and spun down at 4° C. Cells were then plated onto poly-D-lysine coated coverslips or dishes in Neurobasal medium supplemented with 2% B27, 0.25 mM Glutamax, 0.25 mM L-glutamine, penicillin G (50 U/ml), and streptomycin sulphate (50 mg/ml). Cultures were fed every 3-6 days with one half replacement medium without L-glutamine.

For calcium imaging, primary hindbrain neurons seeded on 12 mm coverslips were allowed the appropriate time to form structural networks. These cultures were washed with HBSS and loaded with 2.5 μM FURA-2, AM calcium indicator for 45 minutes at room temperature, according to the manufacturer's protocol. Cells were then washed to remove excess indicator and incubated for 30 minutes to allow internalized esters to become de-esterified. 30 ng/ml of osteocalcin was prepared with the control buffer of 1× HBBS supplemented with 10 mM HEPES buffer and 2 mM CaCl2. Using a Zeiss microscope with a perfusion system, coverslips were first perfused with control and osteocalcin. After each stimulation, cells were depolarized with 50 mM KCl to determine the percentage of live cells being imaged. All treatments were recorded using two-photon laser scanning microscopy by Prairie Technologies and analyzed by Z axis profile plotting using ImageJ.

Brain Explants

Brains were dissected and incubated for 30 minutes in ice cold oxygenated artificial cerebrospinal fluid (ACSF). Brains were then sliced at 500 μm at the midbrain, −1.55 to −2.35 mm from the bregma, and at the level of the brainstem, from −4.04 to −4.48 mm and from −4.60 to −5.20 mm from the bregma, to include the median and dorsal raphe, respectively. These slices were incubated in ACSF for 1 h, constantly oxygenated (95% O2 and 5% CO₂) for 4 h, after which they were treated with either osteocalcin (10 ng/ml) or PBS for four hours. Expression of Tph2, TH, GAD 1, GAD2, and Ddc was measured by qPCR.

Electrophysiology

Brain slice preparations and electrophysiological recordings were performed according to methods known in the art. Briefly, WT mice were anesthetized with ether and then decapitated. The brains were rapidly removed and immersed in an oxygenated bath solution at 40° C. containing (in mM): sucrose 220, KCl 2.5, CaCl₂ 1, MgC1₂ 6, NaH₂PO₄ 1.25, NaHCO₃ 26, and glucose 10 pH 7.3 with NaOH. Coronal slices (350 μm thick) containing dorsal raphe (DR) were cut on a vibratome and maintained in a holding chamber with artificial cerebrospinal fluid (ACSF) (bubbled with 5% CO₂ and 95% O₂) containing (in mM): NaCl 124, KCl 3, CaCl₂ 2, MgCl₂ 2, NaH₂PO₄ 1.23, NaHCO₃ 26, glucose 10, pH 7.4 with NaOH, and were transferred to a recording chamber constantly perfused with bath solution (330 C) at 2 ml/min after at least a 1 hr recovery. Whole-cell current clamp was performed to observe action potentials in DR serotoninergic (5-HT) neurons with a Multiclamp 700A amplifier (Axon instrument, CA). Patch pipettes with a tip resistance of 4-6 MΩ were made of borosilicate glass (World Precision Instruments) with a Sutter pipette puller (P-97) and filled with a pipette solution containing (in mM): K-gluconate (or Cs-gluconate) 135, MgCl₂ 2, HEPES 10, EGTA 1.1, Mg-ATP 2, Na₂-phosphocreatine 10, and Na₂-GTP 0.3, pH 7.3 with KOH. After a giga-Ohm (GΩ) seal and whole-cell access were achieved, the series resistance (between 20 and 40 MΩ) was partially compensated by the amplifier. 5-HT neurons were identified according to their unique properties (long duration action potential, activation by norepinephrine, and inhibition by serotonin itself. Under current clamp, 5-HT neurons were usually quiescent in slices because of the loss of noradrenergic inputs. The application of α1-adrenergic agonist phenylephrine (PE, 3 μM) elicited action potentials and the application of serotonin creatinine sulfate complex (3 μM) inhibited action potentials in these neurons. The effect of leptin on 5-HT neurons was examined in DR neurons responding to both PE and serotonin. Before the application of osteocalcin, action potentials in brainstem neurons were restored by application of PE in the bath. All data were sampled at 3-10 kHz and filtered at 1-3 kHz with an Apple Macintosh computer using Axograph 4.9 (Axon Instruments). Electrophysiological data were analyzed with Axograph 4.9 and plotted with Igor Pro software (WaveMetrics, Lake Oswego, Oreg.).

Physiological Measurements

Physical activity, including ambulatory activity (xamb) and total activity (xtot) was measured using infrared beams connected to the Oxymax system as previously described (Ferron et al, 2012, Bone 50:568-575). Energy expenditure measurements were obtained using a six-chamber oxymax system (Columbus Instruments, Ohio). After 30 hr acclimatation to the apparatus, data for 24 hr measurement were collected and analyzed as recommended by the manufacturer. Oxygen consumption was calculated by taking the difference between input oxygen flow and output oxygen flow. Carbon dioxide production was calculated by taking the difference between output and input carbon dioxide flows. The respiratory exchange ratio (RER) corresponded to the ratio between carbon dioxide production and oxygen consumption (RER=VCO₂/VO₂). Heat production was calculated by indirect calorimetry using the flowing formulas:

Heat=CV×VO₂/BW

CV=3.815+1.232×RER

Behavioral Studies

Tail Suspension Test (TST)

Tail suspension testing was performed as previously described (Mayorga et al., 2001, J. Pharmacology Exper. Therapeutics 298:1101-1107; Stem et al., 1985, Psychopharmacology 85:367-370). Mice were transported a short distance from the holding facility to the testing room and left there undisturbed for at least 1 hour. Mice were individually suspended by the tail (distance from floor was 35 cm) using adhesive tape (distance from tip of tail was 2 cm). Typically, mice demonstrated several escape-oriented behaviors interspersed with temporally increasing bouts of immobility. The parameter recorded was the number of seconds spent immobile. Mice were scored by a highly trained observer, over a 5 min period, blind to the genotype of the mice.

Open Field Paradigm Test (OFT)

Anxiety and locomotor activity of mice were measured using the open field test (David et al., 2009, Neuron 62:479-493). Each animal was placed in a 43×43 cm open field chamber, and tested for 30 min. Mice were monitored throughout each test session by video tracking and analyzed using Matlab software. Mice were placed individually into the center of the open-field arena and allowed to explore freely. The overall motor activity was quantified as the total distance travelled. The anxiety was quantified measuring the number of rearings and the time and distance spent in the center versus periphery of the open field chamber (in %).

Elevated Plus Maze Test (EPMT)

Each mouse was allowed to explore the apparatus for 5 min. Global activity was assessed by measuring the number of entries into the open arms (David et al, 2009, Neuron 62:479-493). Anxiety was assessed by comparing the time spent in the open arms.

Mouse Forced Swim Test (FST)

The forced swimming test was carried out according to the method described by David et al., 2009, Neuron 62:479-493. Briefly, mice were dropped individually into glass cylinders (height: 25 cm, diameter: 10 cm) containing 10 cm water height, maintained at 23-25° C. Animals were tested for a total of 6 min. The total duration of immobility time was recorded. Mice were considered immobile when they made no attempts to escape with the exception of the movements necessary to keep their heads above the water. Mice were scored by an observer blind to their genotypes.

Light and Dark Test

The test was performed in a quiet, darkened room. Mice were individually housed in cages containing a handful of bedding from their home cage and acclimated to the room at least 1 h before the test. Naive mice were placed individually in the testing chamber in the dark compartment. The test was 5 min in duration, and time spent and number of entries in light compartments were recorded a highly trained observer, blind to the genotype of the mice.

Morris Water Maze Test

Spatial memory was assessed with Morris water maze (MWM) setup (Morris, 1981, Nature 297:681-683) using a training protocol adapted for mice (D'Hooge et al., 2005, J. Soc. Neurosci. 25:6539-6549). The maze had a diameter of 150 cm and contained water (23° C.) that was made opaque with non-toxic white paint. The pool was located in a brightly lit room with distal visual cues, including computer, tables and posters with geometric figures attached to the walls. Spatial learning is assessed across repeated trials (4 trials/day for 10 days).

During trials, a small platform (diameter 10 cm) was hidden beneath the surface at a fixed position. Mice were placed in the water at the border of the maze and had to reach the platform after which they were transported back to their home cage. Mice that did not reach the platform within 2 min were gently guided towards the platform and were left on it for 10 s before being placed back in their cages. Four of such daily training trials (inter trial interval: 5 min) were given on 10 subsequent days. Starting positions in the pool varied between four fixed positions (0°, 90°, 180° and 270°) so that each position was used. Since a decrease in latency to find the platform was already present on the second acquisition day, the first acquisition day is also reported.

### Example 2

### Osteocalcin Crosses the Blood Brain Barrier and Binds to Specific Neurons in the Brain

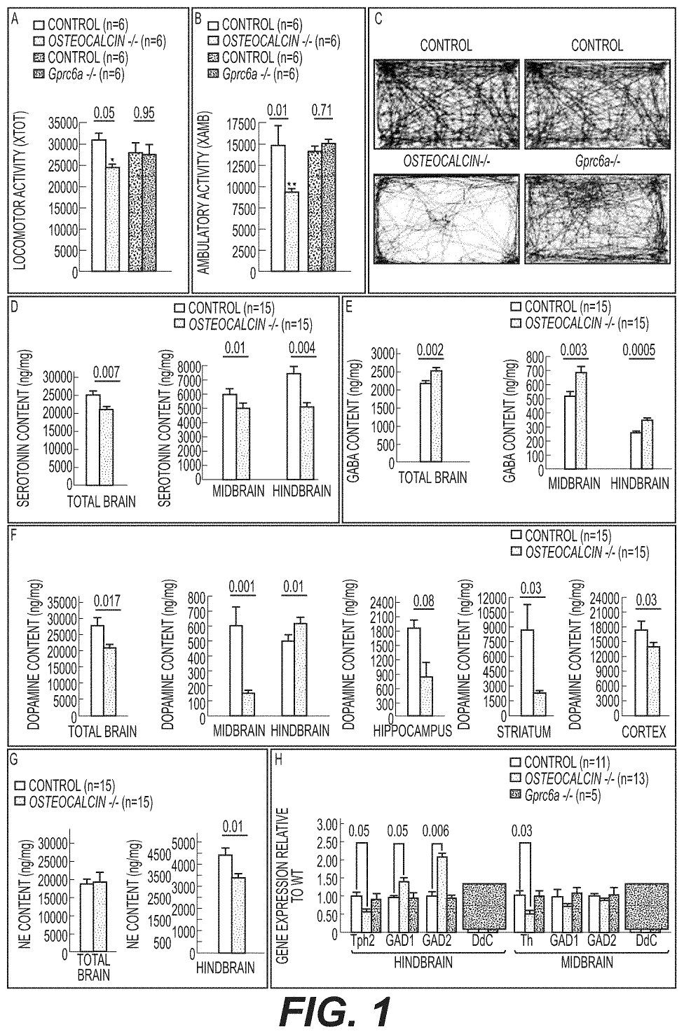

The passivity of Osteocalcin^{-/-} mice is an obvious feature noticed by all investigators handling them. This phenotype was quantified in three-month old Osteocalcin−/− female mice, which demonstrated a significant decrease in locomotor and ambulatory activity during light and dark phases as compared to wild-type (WT) littermates (FIG. 1A-C). Since this observation was made in female mutant mice, it rules out the possibility that this phenotype was secondary to a lack of sex steroid hormones because osteocalcin does not regulate their synthesis in female mice (Oury). Likewise, it was not secondary to a measurable deficit in muscle functions since Osteocalcin−/− and WT mice ran similarly on a treadmill apparatus. This decrease in locomotion was not seen in mice lacking gprc6a, the only known osteocalcin receptor (FIG. 1A-C) and the receptor that is believed to mediate osteocalcin's metabolic functions. This latter result implied that the passivity of the Osteocalcin−/− mice cannot be a consequence of their metabolic abnormalities, since those are equally severe in Osteocalcin−/− and Gprc6a−/− mice.

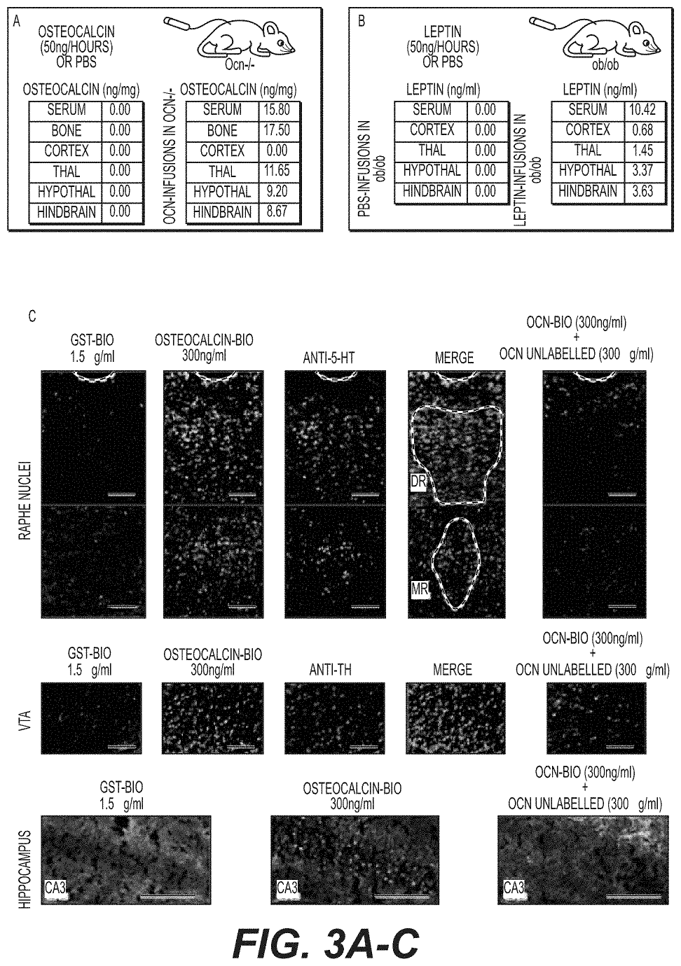

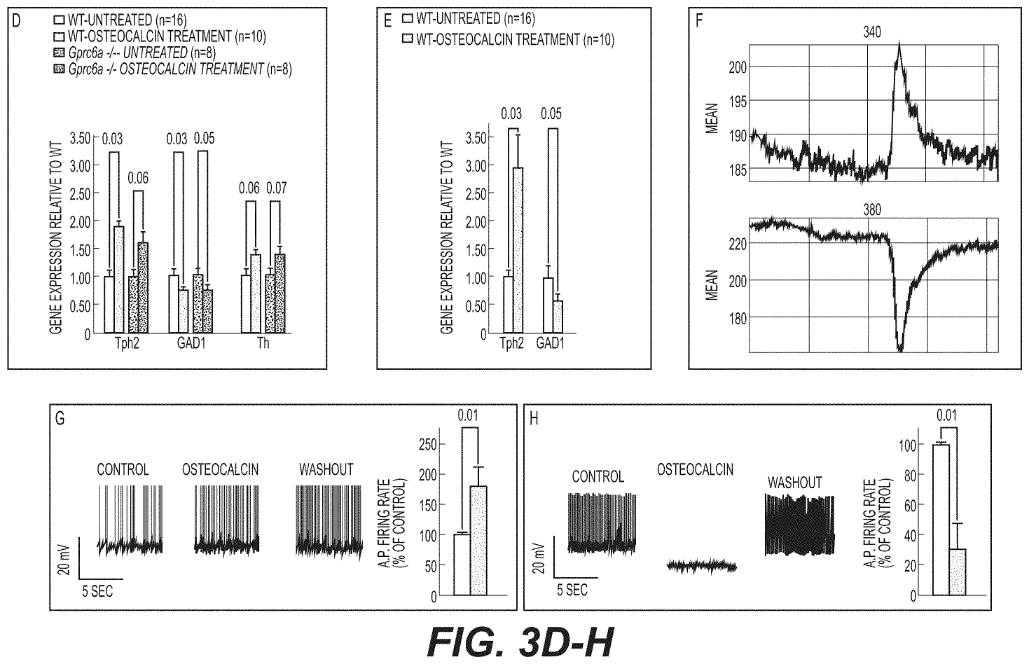

To understand how this behavioral phenotype develops, whether osteocalcin crosses the blood brain barrier (BBB) was tested by installing pumps that subcutaneously delivered vehicle or uncarboxylated osteocalcin (50 ng/hour) in three-month-old Osteocalcin−/− mice. The positive control for this experiment was subcutaneous infusion of leptin (50 ng/hour) in 3 month-old ob/ob mice, since leptin is known to cross the BBB (Banks et al., 1996, Peptides 17:305-311). Seven days later, osteocalcin and leptin content were measured in blood, bone, and in various parts of the brain in Osteocalcin−/− and ob/ob mice, respectively. In ob/ob mice, leptin could be detected in the brainstem and hypothalamus, two structures where it binds (FIG. 3B) (Yadav et al., 2009, Cell 138:976-989, Friedman et al., 2000, Nature 395:763-770). Osteocalcin accumulated in Osteocalcin−/− mice in the brainstem, thalamus, and hypothalamus, where its concentration approached 50% of that observed in serum (FIG. 3A).

This accumulation in discrete regions of the brain raised the question of whether osteocalcin binds to specific neurons in the brain. This was tested by incubating sections of adult or embryonic (E18.5) WT brains with biotinylated undercarboxylated osteocalcin or GST-biotin alone (30 μg/ml), followed by immunofluorescence analysis using an anti-biotin antibody. In the conditions of this assay, osteocalcin bound to several neuronal populations in the forebrain, midbrain, and brainstem (FIG. 3C). In the midbrain, osteocalcin bound to the ventral tegmental area and the substantia nigrae, two nuclei located close to the midline on the floor of the midbrain (FIG. 3C). In the brainstem, osteocalcin bound to neurons of the raphe nuclei (FIG. 3C). Osteocalcin binding in the midbrain and brainstem was specific since it was chased away by an excess of unlabeled osteocalcin but not by an excess of GST (FIG. 3C).

### Example 3

### Osteocalcin Affects the Biosynthesis of Various Neurotransmitters in the Brain

That osteocalcin binds specifically to neurons of the raphe, where brain-derived serotonin is synthesized, together with the influence that brain serotonin exerts on bone mass accrual (Yadav et al., 2009, Cell 138:976-989; Oury et al., 2010, Genes & Development 24:2330-2342), raised the possibility that osteocalcin may influence the synthesis of various neurotransmitters, and that the absence of this regulation may explain the passivity of Osteocalcin^{-/-} mice. The content of serotonin, dopamine, norepinephrine, γ-aminobutyric acid (GABA) and their metabolites in various areas of the brain of three-month old WT and Osteocalcin^{-/-} mice was measured through high pressure liquid chromatography (HPLC).

Serotonin and norepinephrine contents were significantly decreased in the brainstem while dopamine content was markedly decreased in the midbrain, cortex, and striatum of Osteocalcin^{-/-} mice compared to WT mice (FIG. 1D, 1F-G). Of note, this pattern of neurotransmitter accumulation in Osteocalcin^{-/-} mice was similar to what is observed in Tph2^{+/-} mice. Conversely, GABA content was increased in all areas tested in the brains of Osteocalcin^{-/-} mice (FIG. 1E). This is different from what was observed in Tph2^{+/-} mice in which GABA content was increased only in the hindbrain. The content of neurotransmitters was indistinguishable between WT and Gprc6a^{-/-} brains.

The expression of genes encoding rate limiting enzymes implicated in the biosynthesis of these neurotransmitters was studied. Expression of Tph2, the initial and rate limiting enzyme in brain serotonin synthesis, was decreased in the brainstem of Osteocalcin^{-/-} mice and the expression of Th, the rate limiting enzyme in dopamine synthesis, was decreased in the midbrain (FIG. 1H). The same was true for aromatic L-amino decarboxylase (Ddc). Conversely, expression of GAD1 and 2, two enzymes required for GABA biosynthesis, was increased in brainstem of Osteocalcin^{-/-} mice. Expression of all these genes was similar in Gprc6a^{-/-} and WT mice (FIG. 1H), further indicating that osteocalcin signals in the brain in a Gprc6a-independent manner.

A consequence of the positive regulation of Th expression by osteocalcin is that the sympathetic tone as determined by norepinephrine content in the brainstem and Ucp1 expression in brown fat is significantly decreased in Osteocalcin^{-/-} mice. This provides an explanation for the high bone mass originally noted in these mutant mice (Ducy et al., 1996 Nature 382:448-452).

To determine if osteocalcin acts directly on neurons to modulate neurotransmitter synthesis, several types of assays were performed. First, brainstem and midbrain explants from WT and Gprc6a^{-/-} mice were generated. Brains were sliced (500 μm) at the level of the median and dorsal raphe of the brainstem (from −4.04 to −4.48 mm and from −4.60 to −5.20 mm, respectively), so that they would be enriched in serotonin-producing neurons, as well as at the level of substantia nigrae and ventral tegmental areas (VTA) of the midbrain (from −1.55 to −2.35 mm and from −2.55 to −3.25 mm, respectively). Enrichment in serotoninergic and catecholaminergic neurons in these explants was verified by their high Tph2 and Th expression. While leptin, used here as a positive control, reduced, as it should, Tph2 expression in WT or Gprc6a^{-/-} brainstem explants, osteocalcin (3 ng/ml) increased expression of this gene 2.5 fold in both WT and Gprc6a^{-/-} explants. (FIG. 3D). Additionally, osteocalcin increased Th expression in midbrain explants and decreased Gad1 expression in both WT and in Gprc6a^{-/-} hindbrain explants (FIG. 3D). Second, the cultured WT and Gprc6a^{-/-} mouse primary hindbrain neurons (MPHN) were treated with osteocalcin (3 ng/ml) Tph2 expression increased more than three-fold and GAD1 expression decreased by 65% in both WT and Gprc6a−/− primary brainstem neuronal culture following a 2 or 4 hours treatment with osteocalcin (FIG. 3E). Third, to further confirm that osteocalcin signals in neurons of the hindbrain, calcium flux in MPHN treated with undercarboxylated or carboxylated osteocalcin (FIG. 3F) was measured. Undercarboxylated but not carboxylated osteocalcin induced changes in calcium fluxes in those neurons. Finally, an electrophysiological analysis showed, through whole cell current clamp recording, that osteocalcin activates the action potential frequency of brainstem neurons but decreases it in neurons of the locus coeruleus (FIG. 3G). Moreover, osteocalcin inhibits the action potential frequency of the GABAergic interneurons of the hindbrain (FIG. 3H).

Taken together, results of these four different assays support the notion that osteocalcin not only binds to but acts directly, in a Gprc6a-independent manner, on neurons in the raphe to increase Tph2 expression, serotonin accumulation, Th expression, and norepinephrine content, as well as to inhibit GABA synthesis. Osteocalcin also signals in neurons of the midbrain to promote Th expression and dopamine accumulation in that region. Hence, in a feedback manner, bone signals via osteocalcin to serotonergic neurons that are a regulator of bone mass. A consequence of the regulation of Th expression by osteocalcin is that the sympathetic tone is low in Osteocalcin^{-/-} mice, a feature that explains the high bone mass originally noted in these mutant mice (Ducy et al., 1996, Nature 382:448-452).

### Example 4

### Osteocalcin Affects Several Types of Behavior

An implication of the regulation of serotonin and dopamine by osteocalcin is that Osteocalcin^{-/-} mice should demonstrate broad cognitive impairments that, along with their low sympathetic tone, may explain their passivity. To test if this is the case, Osteocalcin^{-/-}, Osteocalcin^{+/-}, Esp^{-/-}, and Gprc6a^{-/-} mice were subjected to a battery of behavioral tests. As controls in these experiments, WT littermates and Tph2^{+/-} mice that demonstrated a decrease in serotonin and dopamine content similar to that one observed in Osteocalcin^{-/-} mice were used.

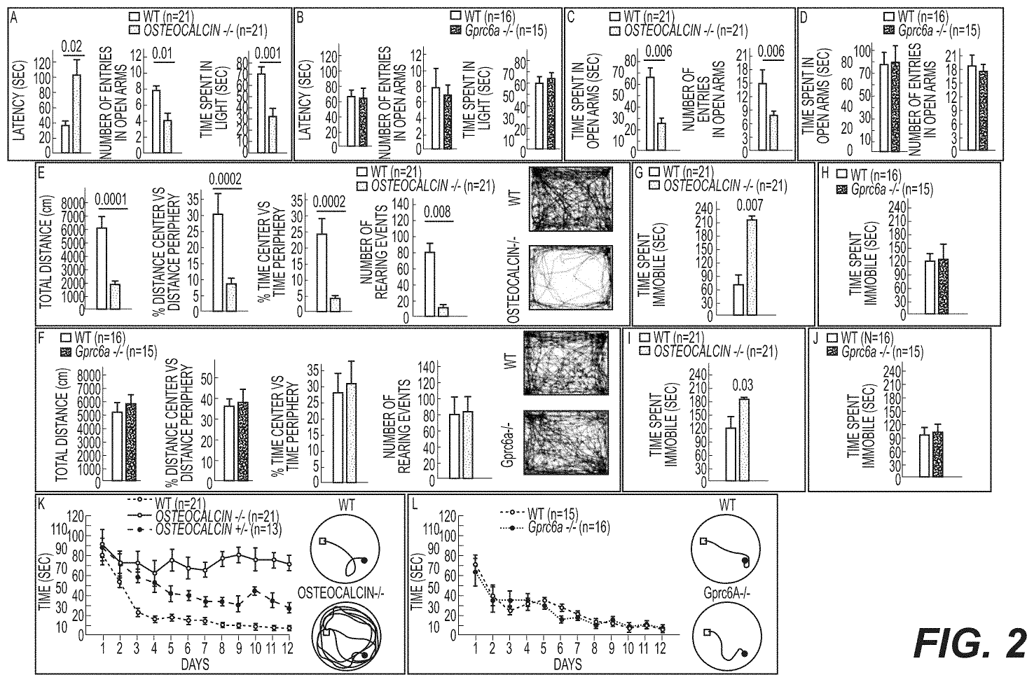

Anxiety-like behavior was analyzed through three conflict-based tests. The first, the dark/light transition test (DLT), is based on the innate aversion of rodents to brightly illuminated areas and on their spontaneous exploratory behavior to avoid the light (Crawley et al., 1985, Neuroscience and Biobehavorial Reviews 9:37-44; David et al., 2009, Neuron 62:479-493). The test apparatus consists of a dark, safe compartment and an illuminated, aversive one. Mice are tested for 6 min each and three parameters recorded: (i) latency to enter the lit compartment, (ii) time spent in the lit compartment, and (iii) number of transitions between compartments. In Osteocalcin−/− mice, there was an increase in the latency to enter in the lit compartment and a decrease of time spent in the lit compartment, two indications of anxiety-related behavior. There was also a decrease in the number of transitions between compartments, another indication of anxiety-related behavior and of motor-exploratory activity (FIG. 2A-B). Conversely, the opposite was true in Esp−/− mice. The elevated plus maze (EPM) test (Lira et al., 2003, Biological Psychiatry 54:960-971; Holmes et al., 2000, Physiology and Behavior 71:509-516) that exploits the aversion of rodents to open spaces was also used. The EPM is comprised of two open and two enclosed arms, each with an open roof elevated 60 cm from the floor. Testing takes place in bright ambient light conditions. Animals are placed onto the central area facing one closed arm and allowed to explore the EPM for 5 min. The total number of arm entries and time spent in open arms measure general activity. A decrease in the proportion of time spent and in the number of entries into the open arms indicates an increase in anxiety. This is exactly what was seen in Osteocalcin^{-/-} mice, while Esp^{-/-} mice demonstrated less anxiety-like behaviors and more exploratory drive than WT littermates (FIG. 2C-D). Lastly, the open field paradigm test (OFT) have been used in which a novel environment evokes anxiety and exploration (David et al., 2009, Neuron 62:479-493; Sahay et al., 2011, Nature 472:466-470). Animals are placed in the center of an open field box and video-tracked under normal light conditions over 30 min. Osteocalcin^{-/-} mice demonstrated a drastic decrease in the distance moved, in time spent in the center, and in vertical activity compared to WT littermates, all features indicative of increased anxiety (FIG. 2E-F).

Anxiety is often accompanied by depression. This was assessed by the tail suspension test (TST), in which animals are subjected to the short-term, inescapable stress of being suspended by their tails, to which they respond by developing an immobile posture (Cryan et al., 2005, Neurosci. Behavorial Rev. 20:571-625; Crowley et al., 2006; Neuropsychopharmacology 29:571-576; David et al., 2009, Neuron 62:479-493). In this test, the more time mice remain immobile, the more depressed they are. This is what was observed in Osteocalcin^{-/-} mice (FIG. 2G-H). In the forced swim test (FST), mice are subjected to two trials during which they are forced to swim in a glass cylinder filled with water from which they cannot escape. The first trial lasts 15 minutes. Twenty-four hours later, a second trial is performed that lasts 6 minutes. Over time, mice cease their attempts to escape and float passively, indicative of a depression-like state. Consistent with the other behavioral tests, Osteocalcin^{-/-} mice spent 45% more time floating than WI mice (FIG. 2I-J).

To assess memory and spatial learning behavior, Osteocalcin^{-/-} and Osteocalcin^{+/-} mice were subjected to the Morris water maze (MWMT) task. This test relies on the ability of mice to learn distance cues and to navigate around the perimeter of an open swimming arena to locate a submerged platform to escape the water. Spatial learning is assessed across repeated trials (4 trials/day for 12 days). Osteocalcin^{+/-} and Osteocalcin^{-/-} mice showed a delayed and a complete inability to learn, respectively (FIG. 2K-L).

As noted for neurotransmitter content and for gene expression in the brain, Gprc6a^{-/-} mice were indistinguishable from WT littermates in all these tests. Collectively, these tests indicate that osteocalcin prevents anxiety and depression, and enhances exploratory behavior, memory, and learning.

### Example 5

### Administration of Osteocalcin Corrects Cognitive Defects

The pharmacological relevance of this ability of osteocalcin to signal in neurons was established by delivering uncarboxylated osteocalcin through intracerebro-ventricular (ICV) infusions (10 ng/hour) in WT and Osteocalcin^{-/-} mice. The localization of the cannula was verified by administering methylene blue through these pumps. The dye labeled all ventricles, indicating that osteocalcin was probably diffusing throughout the brain.

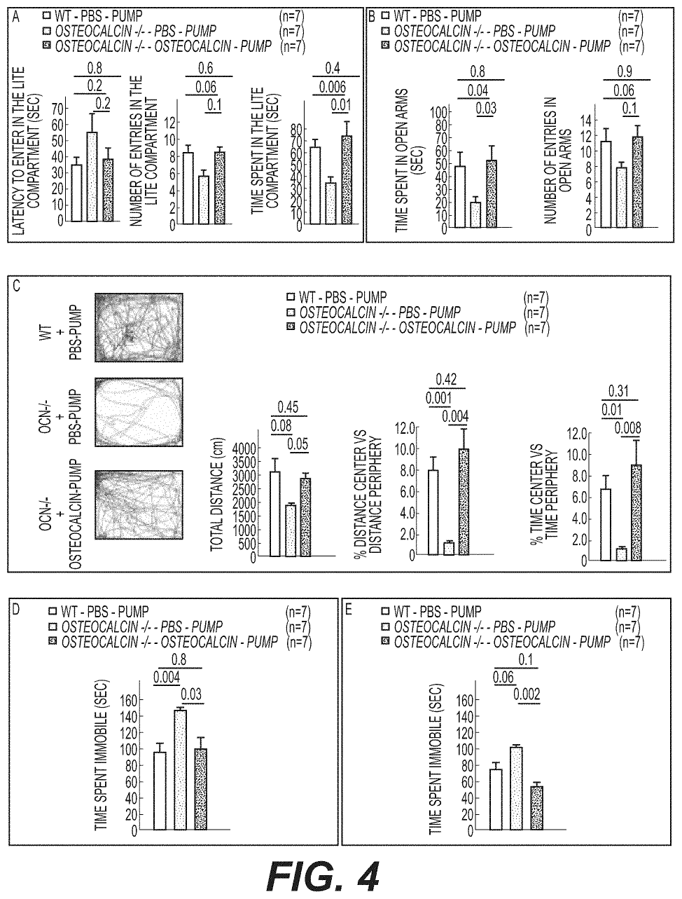

Moreover, measurements of osteocalcin in the blood of infused Osteocalcin^{-/-} mice showed that there was no leakage of the centrally delivered hormone into the general circulation. This week-long treatment with uncarboxylated osteocalcin corrected the anxiety and depression features noted in Osteocalcin^{-/-} mice (FIG. 4A-E). Collectively, the results described herein indicate that osteocalcin prevents anxiety and depression in the mouse by acting directly in the brain.

### Example 6

### Osteocalcin Regulates Cognitive Functions Post-Natally

The results presented above raised the following two questions: Is there a cryptic expression of osteocalcin in the brain that could explain these functions? And, if not, does the influence of osteocalcin on cognitive functions occur post-natally?

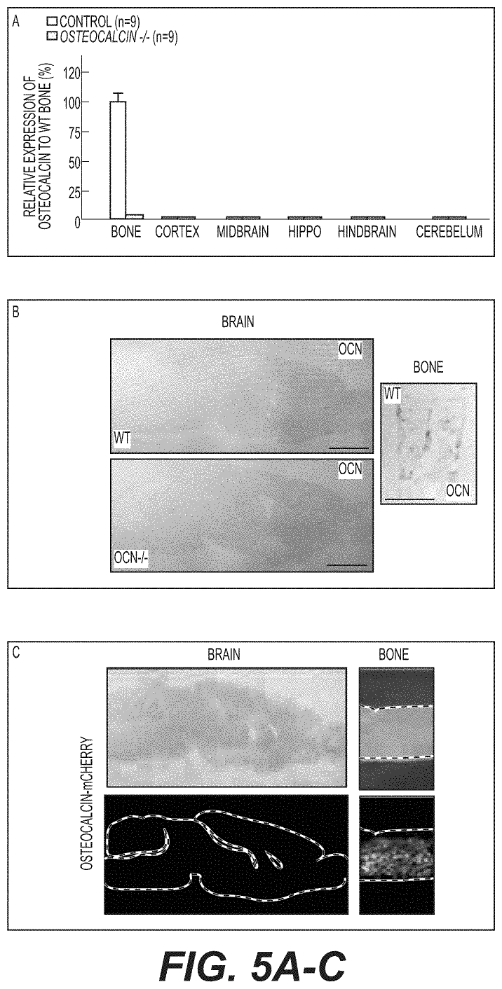

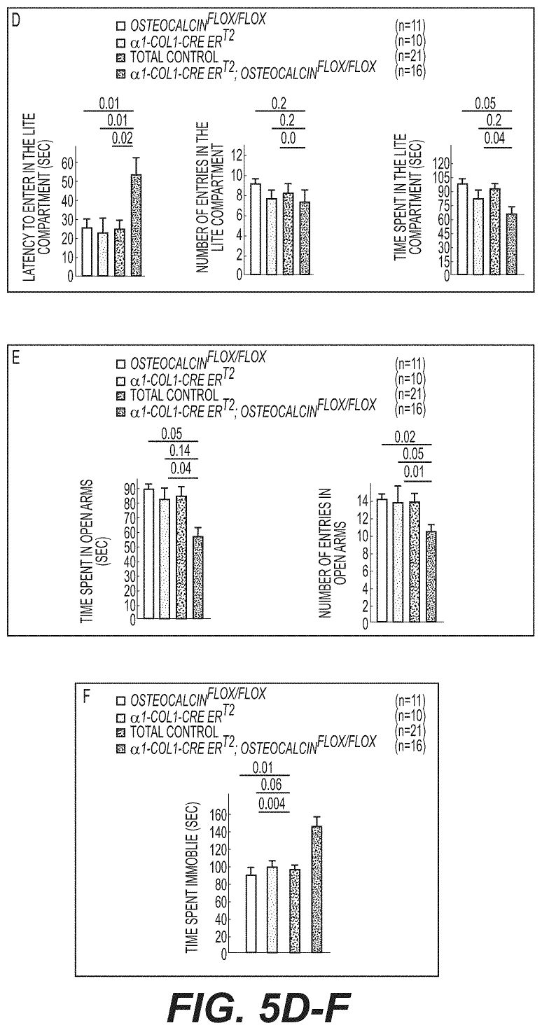

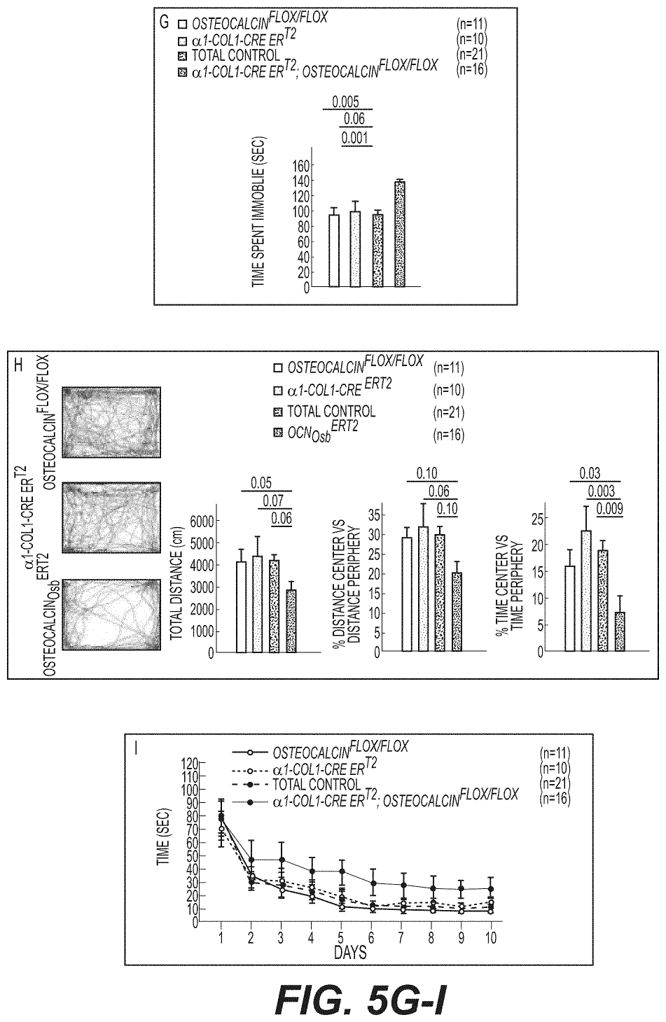

Whether tested by quantitative PCR or in situ hybridization, expression of osteocalcin in the brain of WT adult mice above what was seen in Osteocalcin^{-/-} brain (FIG. 5A-B) was not detected. Moreover, when using a mouse model in which the m-Cherry gene was knocked into the Osteocalcin locus, m-Cherry expression was seen in bone but not in the brain (FIG. 5C). In view of these results, an osteoblast-specific and inducible deletion of osteocalcin was performed by crossing mice harboring a foxed allele of osteocalcin with mice expressing Cre^{ert2} under the control of osteoblast-specific regulatory elements of the mouse Colla1 gene to delete osteocalcin only in osteoblasts (Osteocalcin_{osb}^{ert2-/-} mice). That Osteocalcin_{osb}^{ert2-/-} mice showed a marked reduction in osteocalcin circulating levels following treatment with tamoxifen (1 mg/g BW daily for 5 days) verified that the osteocalcin gene had been efficiently inactivated.

Osteocalcin_{osb}^{ert-/-} mice were treated at 6 weeks with daily injections of tamoxifen (1 mg/20 g of body weight) for 1 week. To ensure that a stable deletion of osteocalcin was achieved, mice were re-injected with another round of tamoxifen every 3 weeks. Six weeks later, α1(I)Collagen-Cre^{ert2}, Osteocalcin^{flox/flox}, and Osteocalcin_{osb}^{ert2-/-} mice were then subjected to behavioral analysis. Tamoxifen-treated Osteocalcin_{osb}^{ert2-/-} mice showed a significant increase in anxiety-like and depression-like behaviors when compared to α1(1)Collagen-Cre^{ert2} or Osteocalcin^{flox/flox} mice (FIG. 5D-I). Spatial learning and memory were also affected in tamoxifen-treated Osteocalcin_{osb}^{ert2-/-} mice but more mildly than in mice harboring a constitutive deletion of Osteocalcin (FIG. 5J). At the molecular level, there was a decrease in Tph2 and Th expression in the brainstem and midbrain respectively of Osteocalcin_{osb}^{ert2-/-} mice treated with tamoxifen and an increase in Gad1 and Gad2 expression in their brainstem (FIG. 5K). These experiments indicate that osteocalcin regulation of anxiety and depression-like behaviors occurs post-natally, while spatial learning and memory seemed to be only partially affected by osteocalcin post-natally.

### Example 7

### Maternal Osteocalcin Crosses the Placenta

Osteocalcin can be measured in the serum of WT embryos as early as E14.5 (FIG. 6A). Studying Osteocalcin expression during development between E13.5 and E18.5 by qPCR or through in situ hybridization failed to detect expression of Osteocalcin anywhere in the embryo except in the developing skeleton (FIG. 6B). Likewise, in the mouse model in which the m-Cherry reporter gene had been knocked into the Osteocalcin locus m-Cherry was expressed in the developing skeleton but not in the developing brain between E13.5 and E18.5. Osteocalcin expression was not detected in the placenta at any of these developmental stages. Hence, during development as is the case after birth, Osteocalcin is a bone-specific gene. The most important result of this survey though was that Osteocalcin expression could not be detected in the developing skeleton until E16.5, two days after the protein is detectable in the blood of the embryos (FIG. 6A-B). This observation suggested that maternal-derived osteocalcin might reach the fetal blood stream.

Any influence of maternal osteocalcin on fetal brain development requires that this hormone cross the placenta. This was investigated through an ex vivo dual perfusion system that monitors the transport of substances across the mouse placenta (Bonnin et al., 2011, Nature 472:347-350; Goeden and Bonnin, 2012, Nature Protocols 8:66-74). This analysis revealed that osteocalcin begins to cross the placenta at day 14.5 of gestation, a developmental stage when Osteocalcin expression cannot be detected in the embryos. A larger transplacental transfer of maternal osteocalcin to the fetal circulation was observed at day 15.5 or 18.5 of gestation (FIG. 6C).

Given the ability of osteocalcin to cross the placenta its circulating levels in embryos of various genotypes and origins were measured. That osteocalcin was detectable (3.6 ng/ml) in the serum of E18.5 Osteocalcin^{-/-} embryos carried by Osteocalcin^{+/-} mothers (FIG. 6D) verified that in vivo maternal osteocalcin crosses the placenta. Still at E18.5, osteocalcin circulating levels in WT embryos were 27.9 ng/ml when carried by WT mothers but only 7.4 ng/ml when their mothers were Osteocalcin^{+/-}. In E16.5 embryos, there were 6.9 ng/ml of osteocalcin in the serum of WT embryos carried by WT mothers while the hormone could not be detected in the serum of WT or Osteocalcin^{+/-} embryos carried by Osteocalcin^{+/-} mothers (FIG. 6D). Osteocalcin also could not be detected at that embryonic stage in Osteocalcin^{-/-} embryos carried by Osteocalcin^{+/-} mothers (FIG. 6D). These results indicate that maternally-derived osteocalcin contributes significantly to the pool of this hormone found in the serum of E16.5 and E18.5 embryos.

### Example 8

### Maternal Osteocalcin Affects Brain Development

To assess the influence of maternal osteocalcin on fetal brain development, an histological analysis of WT, Osteocalcin^{+/-} and Osteocalcin^{-/-} embryos originating from either WT, Osteocalcin^{+/-} or Osteocalcin^{-/-} mothers was performed.

Regardless of the genotype of the mothers, there was no difference in the ratio of brain weight over body weight between WT and Osteocalcin^{-/-} embryos at E16.5 (FIG. 6E). In contrast, this ratio was significantly decreased in E18.5 Osteocalcin^{-/-} embryos originating from Osteocalcin^{-/-} mothers compared to Osteocalcin^{-/-} embryos carried by Osteocalcin^{+/-} mothers or WT embryos carried by WT mothers (FIG. 6E). Consistent with these observations, cresyl violet staining of histological sections showed an enlargement of the cerebral ventricles in the brains of E18.5 Osteocalcin^{-/-} embryos originating from Osteocalcin^{-/-} mothers compared to the ones originating from Osteocalcin^{+/-} mothers (FIG. 6F). When measured by a Tunnel assay, there were significantly more apoptotic cells in the hippocampus of E18.5 Osteocalcin^{-/-} embryos originating from Osteocalcin^{-/-} mothers than in Osteocalcin^{+/-} embryos originating from Osteocalcin^{+/-} mothers or in WT embryos originating from WT mothers (FIG. 6G). A NeuN immunofluorescence study verified that there were fewer neurons in the hippocampus of E18.5 embryos, regardless of their genotype, if they were carried by Osteocalcin^{-/-} mothers than in embryos carried by Osteocalcin^{+/-} mothers (FIG. 6H). There was also a thinning of the molecular layer of the gyms dentate in the hippocampus of adult Osteocalcin^{-/-} mice born from Osteocalcin^{-/-} mothers compared to those born from Osteocalcin^{+/-} mothers. Taken together, these observations indicate that maternal osteocalcin is necessary for proper development of the embryonic mouse brain.

### Example 9

### Maternal Osteocalcin Favors Spatial Memory and Learning in Adult Offspring

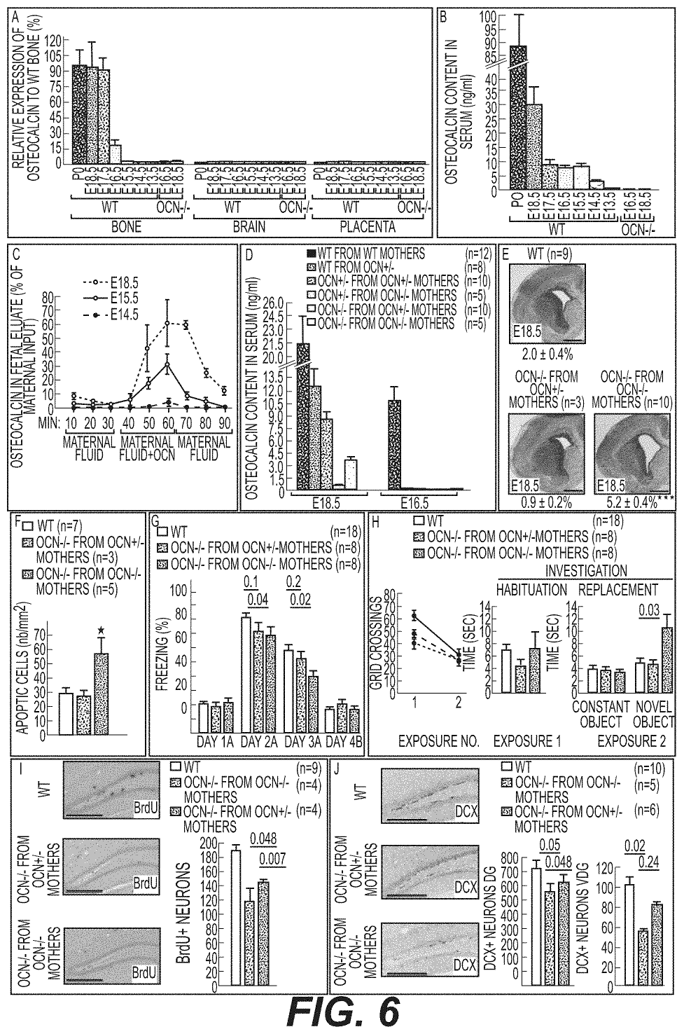

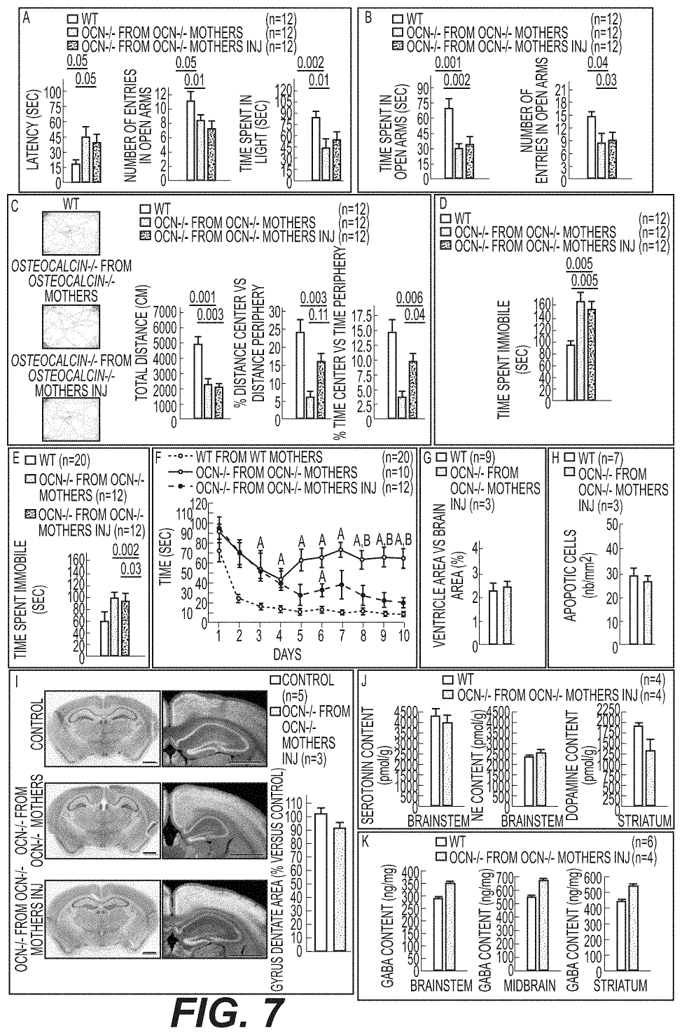

The influence of maternally-derived osteocalcin on fetal brain development raised the question of whether osteocalcin has any influence on cognitive functions in the offspring later in life. To address this question, three month-old Osteocalcin^{-/-} mice born from either Osteocalcin^{-/-} or Osteocalcin^{+/-} mothers were subjected to behavioral tests. While the anxiety and depression-like phenotypes were equally severe in Osteocalcin^{-/-} mice regardless of the genotype of their mothers, the deficit in learning and memory was significantly more severe in Osteocalcin^{-/-} mice born from Osteocalcin^{-/-} mothers than in those born from Osteocalcin^{+/-} mothers (FIG. 7A-F). This result indicated that maternal osteocalcin is needed for the acquisition of spatial learning and memory in adult offspring.

To further evaluate the importance of maternal osteocalcin for the acquisition of spatial learning and memory in adult offspring, pregnant Osteocalcin^{-/-} mothers from E0.5 to E18.5 were treated with injections, once a day, of osteocalcin (240 ng/day). Osteocalcin was never injected in these females or their pups after delivery. This pregnancy-only treatment did not have any beneficial effect on the anxiety or depression phenotypes of the Osteocalcin^{-/-} mice but rescued over two third of their deficit in learning and memory, indicating that this phenotype is, to a large extent, of developmental origin (FIG. 7A-G). Consistent with this observation, cresyl violet staining of histological sections showed a rescue of the cerebral ventricle enlargement in the brains of E18.5 Osteocalcin^{-/-} embryos after injection of the pregnant Osteocalcin^{-/-} mothers (FIG. 7H). Likewise, the number of apoptotic cells was reduced and the number of NeuN positive cells was increased compared to Osteocalcin^{-/-} embryos originating from Osteocalcin^{-/-} mothers that were not injected (FIG. 7I-J). This staining also showed a rescue of the thickness defect in the CA3 and CA4 regions of the hippocampus in adult Osteocalcin^{-/-} originating from Osteocalcin^{-/-} mothers (FIG. 7H). Lastly, a Western blot analysis showed a decrease in Caspase-3 cleaved protein level in the hippocampus of Osteocalcin^{-/-} E18.5 embryos originating from Osteocalcin^{-/-} mothers injected compare to the ones originating from Osteocalcin^{-/-} mothers that were not injected (FIG. 7K).

### Example 10

### Recombinant Osteocalcin

Recombinant osteocalcin was bacterially produced and purified on glutathione beads according to standard procedures. Osteocalcin was then cleaved from the GST subunit using thrombin digestion. Thrombin contamination was removed using an affinity column. The purity of the product was qualitatively assessed by SDS-PAGE. Bacteria do not have a gamma-carboxylase gene. Therefore, recombinant osteocalcin produced in bacteria is always completely undercarboxylated at all three sites.

### Example 11

### Direct Delivery of Osteocalcin to the Brain Improves Cognitive Function in Wild-Type (WT) Adult Mice in a Dose Dependent Manner

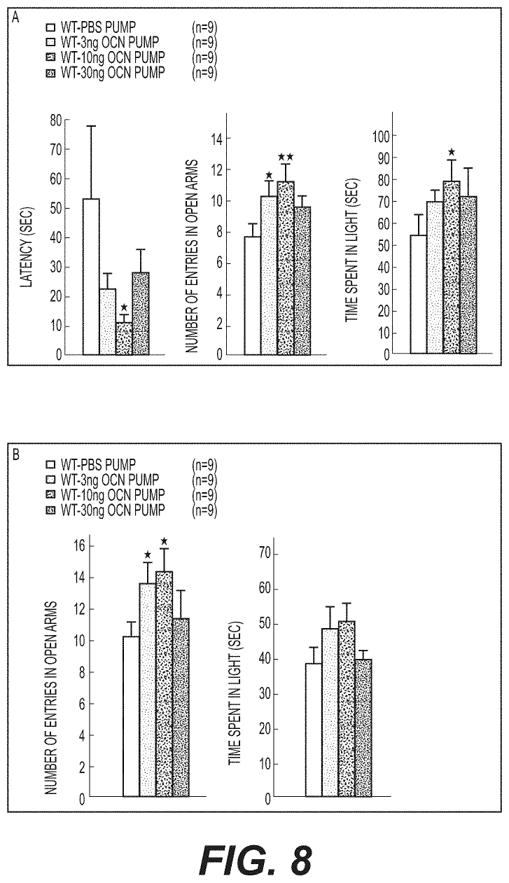

To determine if osteocalcin is sufficient to improve cognitive function in adult mice, WT 2-month old mice were implanted with ICV pumps delivering vehicle (PBS), or 3, 10, or 30 ng/hr recombinant uncarboxylated full-length mouse osteocalcin for a period of one month. After one month of infusion, animals were subjected to behavioral testing. Based on their performance in the dark to light transition (D/LT) test and the elevated plus maze (EPMT) test, animals receiving 3 or 10 ng/hour of recombinant uncarboxylated full-length mouse osteocalcin showed a decrease in anxiety-like behavior. This improvement is evidenced by an increase in the exploration of the lit compartment and open arms in the D/LT and EMP tests, respectively (FIG. 8A-B).

### Example 12

### Direct Delivery of Osteocalcin to the Brain of Aged Wild Type (WT) Mice Improves Hippocampal Functions

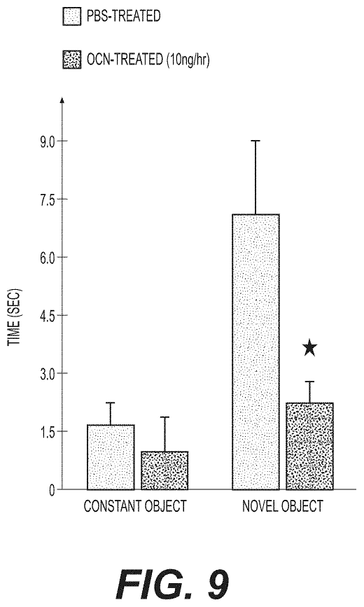

ICV pumps delivering (10 ng/hr) recombinant uncarboxylated full-length mouse osteocalcin were implanted in 16 month old WT mice. After an infusion period of one month, the mice were subjected to a modified version of the Novel Object Recognition test, to assay memory and hippocampal function. Briefly, mice were given five 5 minute exposures, with 3 minute resting intervals between exposures, to a novel arena containing two objects. During exposures 1-4, mice were habituated to these two objects, which elicited equal amounts of exploration. In the fifth exposure, one of the objects was replaced with a novel object. Aged mice receiving either PBS or recombinant uncarboxylated full-length mouse osteocalcin were both able to discriminate between the novel and constant objects. However, FIG. 9 shows that mice which had received osteocalcin treatment spent less time exploring the novel object than mice treated with vehicle alone, indicating improved efficiency in hippocampal context encoding and/or acquisition efficiency (Denny et al., 2012, Hippocampus 22:1188-1201).

### Example 13

### Osteocalcin is Necessary and Sufficient for CREB Phosphorylation in the Hippocampal

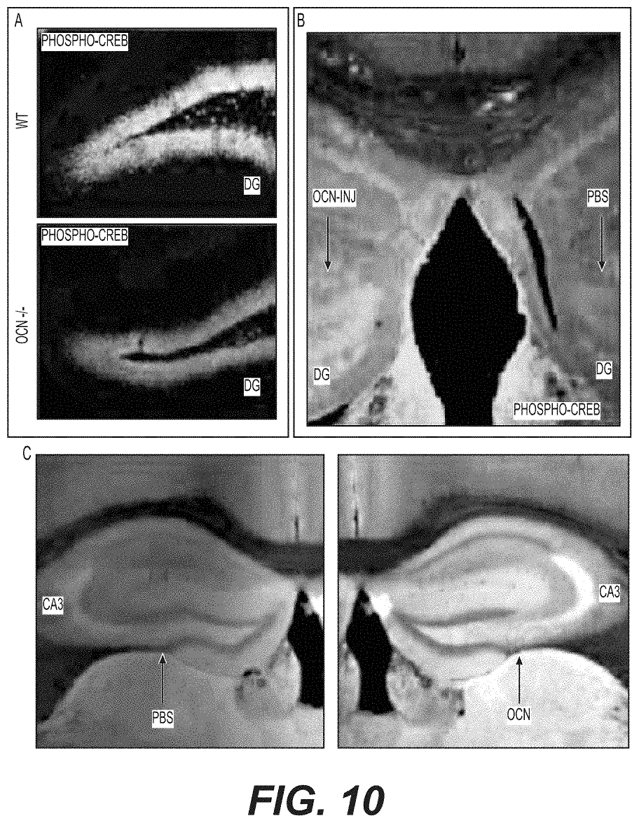

The resulting effects on animal behavior of direct recombinant uncarboxylated osteocalcin delivery raise the question of the molecular mechanism of action of osteocalcin in the brain. Given that osteocalcin acts through a G-protein coupled receptor pathway in other tissues, e.g., pancreas and testis, the phosphorylated CREB levels in the hippocampi of Ocn−/− and WT animals was checked. It was observed that pCREB staining is dramatically decreased in the dentate gyms (DG) of the hippocampus in Ocn−/− animals (FIG. 10A). The hippocampus is essential for optimal spatial learning and memory in rodents. It was then asked whether the acute stereotactic injection of 10 ng of recombinant uncarboxylated osteocalcin directly into the hippocampus of WT animals would affect pCREB levels. At 16 h post injection, pCREB staining was increased in the hemisphere injected with osteocalcin versus the one injected with PBS in the same animal (FIG. 10B). Moreover, a widespread and dramatic increase in PKA staining, known to lead to CREB activation, was observed in the injected hemisphere at the 16 h post injection timepoint (FIG. 10C).

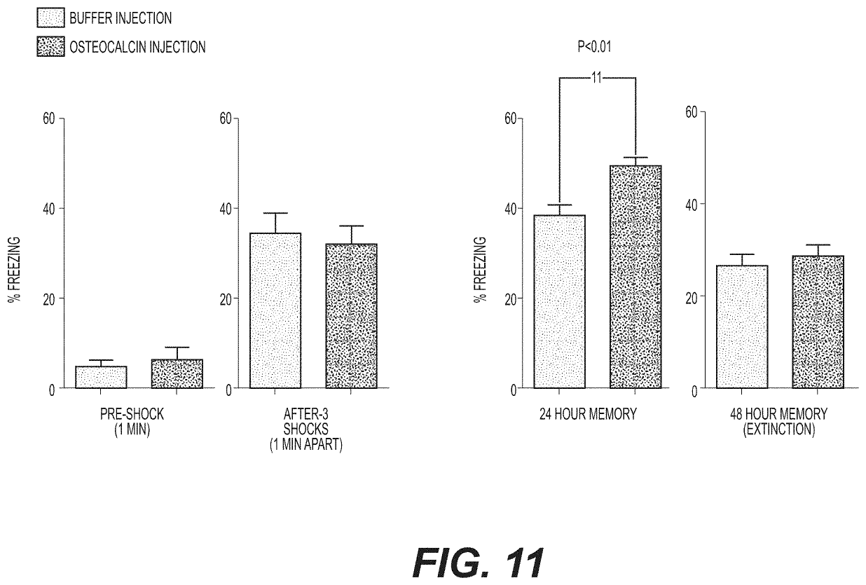

To determine whether these acute injections and corresponding activation of the CREB pathway are functionally relevant, Contextual Fear Conditioning (CFC), a hippocampus dependent task that assesses long term memory, was performed. Mice were injected acutely in both hemispheres with either PBS or 10 ng recombinant uncarboxylated osteocalcin. Mice injected with osteocalcin (n=4 per group) displayed increased freezing behavior as compared to controls (FIG. 11), indicating that just one dose of osteocalcin improved long term memory recall.

### Example 14

### GPR158 is the Brain Osteocalcin Receptor

Materials and Methods

Animals and Sample Size

Gpr158^{-/-} (Gpr158^{tm1(KOMP)Vlcg}) mice were purchased from KOMP repository (VG10108). Compound heterozygous mice (Gpr158^{+/-}, Ocn^{+/-} and Gpr158^{+/-}; Ocn^{+/-}) were maintained on a 129-Sv/C57/BL6 mixed genetic background. Ocn^{-/-}, Ocn^{+/-} and Runx2^{+/-} have been previously described (Ducy, P., et al., 1996, Nature 382:448-452; Ducy, P., et al., 1997, Cell 89:747-754. Runx2^{+/-} were maintained on a C57/BL6 background. For all experiments, littermates have been used as controls. Females were used in all experiments unless otherwise stated. Stereotaxic surgery was performed in 3 monthold C57BL/6J male mice obtained from Janvier Laboratory stock. Osmotic pumps, plasma injection and alendronate injection experiments were performed in 12 month-, 16 month and 3 month-old 129-Sv mice obtained from Taconic biosciences. After arrival, the mice were housed at least 2 weeks, five animals per cage (polycarbonate cages (35.5×18×12.5 cm)), under a 12 hr light/dark cycle with ad libitum access to food and water before experiments. All experiments involving animals were approved by the Institutional Animal Care and Use Committee of Columbia University Medical Center.

Plasma Collection

Pooled mouse plasma was collected from young (3 months) WT or Ocn^{-/-} or aged (16 months) mice by intracardial bleed at time of euthanasia. Plasma was prepared from blood collected with EDTA into Capiject T-MQK tubes followed by centrifugation at 1,000 g for 10 minutes. All plasma aliquots were stored at −80 ° C. until use. Before administration, plasma was dialyzed using 3.5-kDa D-tube dialyzers (EMD Millipore) in PBS to remove EDTA. Young adult mice were systemically treated with plasma (100 μl per injection) by injections into the tail vein eight times over 24 days.

Stereotaxic Surgery

Mice were anesthetized with intra-peritoneal injection of 20 mg/ml BW ketamine hydrochloride (1000 Virbac) and 100 mg/ml BW xylazine (Rompun 2%; Bayer) and placed in a stereotaxic frame (900SL-KOPF). Ophthalmic eye ointment was applied to the cornea to prevent desiccation during surgery. The area around the incision was trimmed and Vetedine solution (Vetoquinol) was appplied. Lentiviruses expressing shRNA targeting Gpr158 or non-effective scramble shRNA in pGFP-C-shLentiVector were injected bilaterally into the anterior hippocampi using the following coordinates: (from bregma) anterior=−2.0 mm, lateral=+/−1.4 mm and height=-1.33 mm. Coordinates were determined using the Mouse Brain in Stereotaxic Coordinates (Paxinos and Franklin, 2008). Two weeks later, osteocalcin (10 ng) or NaCl were injected using the same coordinates. The lentiviruses or osteocalcin were injected stereotaxically using a 10 μl Hamilton syringe (1701RN) over either 12 or 4 min (injection speed: 0.25 μl per min), respectively. To limit reflux along the injection track, the needle was maintained in situ for 4 min between each 1 μl. Then, the skin was closed using silk suture and the mice were injected locally with surgical analgesic (ketoprofen).

Drugs Treatment

For plasma injection experiments, 100 μl of plasma were injected 8 times during 24 days, via tail vein. Each group described is represented individually in each panel. For osteocalcin delivery in WT mice, pumps (Alzet micro-osmotic pump, Model 1002) delivering osteocalcin (30 ng/hr for 12 month-old and 90 ng/hr for 16 month-old mice), or vehicle, were surgically installed subcutaneously in the back of mice. For alendronate delivery to 3 month-old mice, intraperitoneal injections (40 μg/kg) were performed twice a week for 6 weeks. During the last 4 weeks of treatment and during behavioral testing, half of the alendronate-treated mice received 60 ng/hr osteocalcin by osmotic pump. Control mice receiving vehicle or mice receiving alendronate were implanted with osmotic pumps delivering vehicle only.

Bilateral stereotaxic injections in the anterior hippocampus (using the following coordinates from bregma: X=−2.0 mm, Y=+/−1.4 mm and Z=−1.33 mm) were performed with 3 μl of lentiviruses expressing shRNA against Gpr158 (titer: 3,4 ′ 109 GC/ml) or scramble shRNA (1,4 ′ 109 GC/ml) cloned in pGFP-C-shLentiVector (Origene, Rockville USA). Two weeks later local bilateral stereotaxic injections of osteocalcin (10 ng/μl) or NaCl (at a volume of 1 μl) were performed in the anterior hippocampus (using the same coordinates previously described). Next, the mice were subjected to the training phase (NOR and CFC) 12 h after the stereotaxic injections of osteocalcin, and to the testing phase 24 h following the habituation phase.

Behavioral Studies

All animals of the same batch were born within an interval of 2 weeks and were kept in mixed genotype per group of 5 females in the same cage, at standard laboratory conditions (12 h dark/light cycle, constant room temperature and humidity, and standard lab chow and water ad libitum). For each test, the mice were transported a short distance from the holding mouse facility to the testing room in their home cages or in the transport boxes filled with bedding from their home cages. Behavioral testing of the mice was performed on a battery of functional tests between 3 and 16 months-of age, and mouse weight was between 22 g and 32 g. The tests were performed by an experimentalist blind to the genotypes or treatment of the mice under study.

Elevated plus maze test (EPMT): This test takes advantage of the aversion of rodents to open spaces. The EPM apparatus comprises two open and two enclosed arms, each with an open roof, elevated 60 cm from the floor (Holmes, A., et al., 2000, Physiology & behavior 71:509-516; Lira, A., et al., 2003, Biological psychiatry 54:960-971). Testing takes place in bright ambient light conditions. Animals are placed into the central area facing one closed arm and allowed to explore the EPM for 6 min. The total number of arm entries and time spent in open arms is recorded. An increase in anxiety is indicated by a decrease in the proportion of time spent in the open arms (time in open arms/total time in open or closed arms), and a decrease in the proportion of entries into the open arms.

Light to dark transition test: This test is based on the innate aversion of rodents to brightly illuminated areas and on their spontaneous exploratory behavior in response to the stressor that light represents. The test apparatus consists of a dark, safe, compartment and an illuminated, aversive, one. Mice were tested for 6 min and two parameters were recorded: (i) latency to enter the lit compartment, (ii) time spent in this compartment, an index of the anxiety-related behavior and (iii) number of transitions between compartments, an index of anxiety-related behavior as well as exploratory activity.

Open field (OFT): This test takes advantage of the aversion of rodents to brightly lit areas (David, D. J., et al., 2009, Neuron 62:479-493). Each mouse is placed in the center of the OFT chamber (a white 43×43 cm chamber) and allowed to explore for 30 min. Mice were monitored throughout each test session by video tracking and analyzed using Autotyping (Patel, T. P., et al., 2014, Front Behav Neurosci 8:349). The overall motor activity was quantified as the total distance traveled (ambulation). Anxiety was quantified by measuring the time spent in the center of the OFT chamber.

Morris water maze test: Animals are transported to the testing room in their home cages, and left undisturbed for at least 30 minutes prior to the first trial. The maze is comprised of a large swimming pool (150cm diameter) filled with water (23° C.) made opaque with non-toxic white paint. The pool is located in a brightly lit room filled with visual cues, including geometric figures on the walls of the maze demarking the four fixed starting positions of the trials, at (12:00, 3:00, 6:00 and 9:00). A 15 cm round platform is hidden 1 cm beneath the surface of the water at a fixed position. Each daily trial block consisted of four swimming trials, with each mouse starting from the same randomly chosen starting position. The starting position is varied between days. On day 1, mice that fail to find the platform within 2 min are guided to the platform. They must remain on the platform for 15 s before they are returned to their home cage. Mice are not guided to the platform after day 1, and the time it takes them to reach the platform over repeated trials (3 trails/day for the next 10 days) is recorded as a measure of spatial learning.

Novel object recognition test (NOR): The NOR paradigm assesses the rodent's ability to recognize a novel object in the environment. The NOR task will be conducted, as previously described7, in an opaque plastic box using 2 different objects: (1) a clear plastic funnel (diameter 8.5 cm, maximal height 8.5 cm) and (2) a black plastic box (9.5 cm³). These objects elicit equal levels of exploration as determined in pilot experiments (Denny, C. A., et al., 2012, Hippocampus 22:1188-1201; Oury, F., et al., 2013, Cell 155:228-241). The NOR paradigm consists of 3 exposures over the course of 3 days. On day 1, the habituation phase, mice are given 5 minutes to explore the empty arena, without any objects. On day 2, the familiarization phase, mice are given 10 minutes to explore 2 identical objects, placed at opposite ends of the box. On day 3, the test phase, mice are given 15 minutes to explore 2 objects, one novel object and a copy of the object from the familiarization phase. The object that serves as the novel object (either the funnel or the box) as well as the left/right starting position of the objects are counterbalanced within each group. Mice are placed in the center of the arena at the start of each exposure. Between exposures, mice are held individually in standard cages, the objects and arenas cleaned, and the bedding replaced. Preference for the novel object is assessed based on the fraction of time that a mouse spends exploring the novel object compared to the familiar object. Exploration is scored from video recordings of each exposure and recorded using the Stopwatch program. An equal exploration time for the two objects, or a decreased percentage of time spent with the novel object compared to WT controls indicates impairment in hippocampal memory.

Contextual fear conditioning: The conditioning apparatus consisted of two sound and light attenuated conditioning boxes (67×55×50 cm, Bioseb, France), and mice were run individually in the conditioning boxes. Each box was constructed from black methacrylate walls and a Plexiglas front door. Floor of the chamber consisted of 27 stainless steel bars (3 mm in diameter, spaced 7 mm apart (center-to-center) wired to a shock generator with scrambler for the deliveiy of foot shock. Signal generated by the mice movement was recorded and analyzed through a high sensitivity weight transducer system, The analogical signal was transmitted to the Freezing software module through the load cell unit for recording purposes and posterior analysis in terms of activity/immobility (Freezing). An additional interface associated with corresponding hardware allowed controlling the intensity of the shock from the Freezing software. The fear conditioning procedure took place over two consecutive days. On day 1, mice were placed in the conditioning chamber, received 3 foot-shocks (1 sec, 0.5 mA) which were administered at time points of 60, 120 and 180 sec after the animals were placed in the chamber. They were returned to their home cage 60 sec after the final shock. Contextual fear memory was assessed 24 hr after conditioning by returning the mice to the conditioning chamber and measuring freezing behavior during a 4 min retention test. Freezing was scored and analyzed automatically using Packwin 2.0 software (bioseb, France). Freezing behavior was considered to occur if the animal froze for at least a period of two seconds. All the CFC procedures and the data analyses were performed by two independent experimentators blinded to the treatment.

Bone Histomorphometry

Lumbar vertebrae or tibia dissected from 3 month-old female mice were fixed for 24 h, dehydrated with graded concentrations of ethanol, and embedded in methyl methacrylate resin according to standard protocols. Von Kossa/ Van Gieson, toluidine blue, and tartrate-resistant acid phosphatase stainings were used to measure bone volume over tissue volume (BV/TV). Vertebrate pictures for Von Kossa/Van Gieson were obtained using a microscope (DM4000B; Leica) equipped with a camera (DFC300 FX; Leica) using a 2.5× magni cation. Images were acquired with Fire soft- ware (Leica), and BV/TV was analyzed using ImageJ software (National Institutes of Health).

Electrophysiology

Coronal brain slices containing the hippocampus were prepared from wild type and KO mice (3-4 weeks old, male) as previously reporte. Briefly, mice were anesthetized with isoflurane and then decapitated to harvest brains, which were rapidly removed and immersed in an oxygenated cutting solution at 4° C. containing (in mM): sucrose 220, KCl 2.5, CaCl2 1, MgCl2 6, NaH2PO4 1.25, NaHCO3 26, and glucose 10, and adjusted to pH 7.3 with NaOH. Coronal slices containing the hioopcampus (300 μm thick) were cut with a vibratome, trimmed to contain just the hippocampus. After preparation, slices were stored in a holding chamber with an oxygenated (with 5% CO2 and 95% O2) artificial cerebrospinal fluid (ACSF) containing (in mM): NaCl 124, KCl3, CaCl2 2, MgCl2 2, NaH2PO4 1.23, NaHCO3 26, glucose 10, pH 7.4 with NaOH. The slices were eventually transferred to a recording chamber constantly perfused with ACSF at 33° C. at a rate of 2 ml/min after at least a 1 hour recovery in the storage chamber. Whole-cell current clamp was performed to observe spontaneous action potentials (APs) in visually identified pyramidal neurons in the CA3 area of the hippocampus with a Multiclamp 700 A amplifier (Molecular devices, Sunnyvale, CA). The patch pipettes with a tip resistance of 4-6 Ma were made of borosilicate glass (World Precision Instruments, Sarasota, FL) with a pipette puller (Sutter P-97) and back filled with a pipette solution containing (in mM): K-gluconate 135, MgC12 2, HEPES 10, EGTA 1.1, Mg-ATP 2, Na2-phosphocreatine 10, and Na2-GTP 0.3, pH 7.3 with KOH. After a stable base of APs were recorded for 10 minutes, osteocalcin was applied to the recorded cells through bath application at a concentration of 10 ng/ml for 5-10 minutes and then washed out with ACSF. All data were sampled at 10 kHz and filtered at 6 kHz with an Apple Macintosh computer using Axograph X (AxoGraph Scientific, Sydney, Australia). Action potentials were detected and analyzed with AxoGraph X and plotted with Igor Pro software (WaveMetrics, Lake Oswego, Oreg.).

Real-Time RNA Transcript Determination

All dissections were performed in ice-cold PBS 1× under a Leica MZ8 dissecting light microscope. Brainstems were isolated from the cerebellum and the hypothalamus and removed from the midbrain during collection. All parts of the brain isolated were snap frozen in liquid nitrogen and kept at −80° C. until use.

RNA was isolated from brain tissue using TRIZOL (Invitrogen). cDNA synthesis was performed following standard protocol, q-PCR analyses were done using specific quantitative PCR primers (sequences available upon request), and expressed relative to Gapdh levels.

Primary Hippocampal Culture

Hippocampal neurons were isolated from mouse embryos (embryonic day 16.5). After dissection, hippocampi were digested in Tryspin 0.05% and EDTA 0.02% for 15 minutes at 37° C. After 3 wash in DMEM (high glucose and sodium pyruvate) supplemented with 10% of fetal bovine serum, 100 U/mL Penicillin-Streptomycin and 1× GlutaMAX, cells were dissociated by pipetting up and down and then plated. After the culture was established, medium was changed 2 times per week with Neurobasal medium supplemented with B-27 supplement and 1× GlutaMAX. Experiments were performed on cells after 15 days of culture (DIV 15).

Biochemistry and Molecular Biology

For Western blotting, frozen hippocampi from adult mice were lysed and homogenized in 250 □l tissue lysis buffer (25 mM Tris HCl 7.5; 100 mM NaF; 10 mM Na4P2O7; 10 mmM EDTA; 1% NP 40). Proteins were transferred to nitrocellulose membranes, and blocked with TBST-5% BSA for 1 hour. Antibodies: anti-Runx2 M-70 sc-10758, Santa Cruz, anti-BDNF: sc-546, Santa Cruz; anti-tubulin: T6199, Sigma; anti-Gpr158 ABIN1535721, Assay Biotechnology ; anti-Na,K ATPase 3010S Cell Signaling , were diluted (1:1000) in TBST-5% BSA and incubated overnight at 4° C. HRP-coupled secondary antibodies and ECL were used to visualize the signal. Western blot bands were quantified using ImageJ software. cAMP accumulation was measured in primary hippocampal neurons by using cAMP Parameter Assay Kit (R&D systems) and performed in primary hippocampal neurons (DIV15) following manufacturer instructions. For IP1 accumulation was determined in primary hippocampal neurons (DIV15) by using IP-One ELISA assay kit (Cisbio) following manufacturer instructions. Pulldown of Gpr158 was performed in solubilized membrane from Ocn−/− hippocampi using standard procedures. Briefly, hippocampi were dissected on ice and homogenized in buffer A (10 mM Tris-HCl pH 7.4, 320 mM sucrose and protease inhibitors) with a Glass/Teflon Potter Elvehjem homogenizer (20 strokes). Homogenized hippocampi were centrifuged at 3000 g for 10 minutes at 4° C. Then, supernatants were ultra-centrifuged at 40000 g for 20 minutes 4° C. Pellets were resuspended in Buffer A supplemented with 150mM NaCl and 1% n-Octyl β-D-thioglucopyranoside. Solubilized membranes were diluted in buffer A supplement with 150mM NaCl and 0.2% n-Octyl β-D-thioglucopyranoside. For the pulldown, biotinylated osteocalcin (7 ug) was incubated for different time points at 4° C. Thirty microliters of Dynabeads M-280

Streptavidin were added for 30 minutes at room temperature followed by PBS washes. Purified proteins were eluted from the beads by adding Laemli protein buffer and heated at 65° C. for 15 minutes. For hormonal measurement; circulating levels of the carboxylated, undercarboxylated or uncarboxylated forms of osteocalcin were measured by ELISA. CTX content in serum (ng/ml) were measured with specific ELISAs (RatLaps™ (CTX-I) EIA (Immunodiagnosticsystems).

In Situ Hybridization

In situ hybridization was performed using 35S-labeled riboprobe as described (Ducy, P., et al., 1997, Cell 89:747-754). The Gpr158, Th, Gp156, Gpr179, Gprc5a, Gprc5b, Gprc5c, Gprc5d probe is each 3' UTR. Hybridizations were performed ovenight at 57° C., and washes were performed at 63° C.

What is claimed is:

1. A method of treating or preventing a cognitive disorder in a mammal comprising administering to a mammal in need thereof a pharmaceutical composition comprising a therapeutically effective amount of an activator of GPR158 and a pharmaceutically acceptable carrier or excipient.

2. The method of claim 1 wherein the mammal is a human.

3. The method of claim 2 wherein the cognitive disorder is selected from the group consisting of cognitive loss due to neurodegeneration associated with aging, anxiety, depression, memory loss, learning difficulties, and cognitive disorders associated with food deprivation during pregnancy.

4. The method of claim 3 wherein the cognitive disorder is anxiety due to aging, depression due to aging, memory loss due to aging, or learning difficulties due to aging.

5. The method of claim 2 or 3 wherein the activator is a small molecule, a peptide, an antibody, or a nucleic acid.

6. The method of claim 3 wherein the cognitive disorder is anxiety.

7. The method of claim 3 wherein the cognitive disorder is depression.

8. The method of claim 3 wherein the cognitive disorder is memory loss.

9. The method of claim 3 wherein the cognitive disorder is learning difficulties.

10. A method of diagnosing and treating a cognitive disorder in a patient comprising: (i) determining a patient level of undercarboxylated/uncarboxylated osteocalcin in a biological sample taken from the patient;
(ii) comparing the patient level of undercarboxylated/uncarboxylated osteocalcin and a control level of undercarboxylated/uncarboxylated osteocalcin, and
(iii) if the patient level is significantly lower than the control level, administering to the patient a therapeutically effective amount of an activator of GPR158.

11. Use of a pharmaceutical composition comprising an activator of GPR158 for treating or preventing a cognitive disorder in a mammal.

12. The use of claim 11 wherein the mammal is a human and the osteocalcin is human osteocalcin.

13. The use of claim 11 wherein the cognitive disorder is selected from the group consisting of cognitive loss due to neurodegeneration associated with aging, anxiety, depression, memory loss, learning difficulties, and cognitive disorders associated with food deprivation during pregnancy.

14. The use of claim 11 wherein the cognitive disorder is anxiety due to aging, depression due to aging, memory loss due to aging, or learning difficulties due to aging.

15. The use of claim 11 wherein the cognitive disorder is anxiety.

16. The use of claim 11 wherein the cognitive disorder is depression.

17. The use of claim 11 wherein the cognitive disorder is memory loss.

18. The use of claim 11 wherein the cognitive disorder is learning difficulties.
